(19) United States
(12) Patent Application Publication
(10) Pub. No.: US 2022/0204457 A1
(43) Pub. Date: Jun. 30, 2022

(54) ARGININE GINGIPAIN INHIBITORS

(71) Applicant: Cortexyme, Inc., South San Francisco, CA (US)

(72) Inventors: Robert A. Galemmo, South San Francisco, CA (US); Andrei W. Konradi, South San Francisco, CA (US); Casey C. Lynch, South San Francisco, CA (US); Stephen S. Dominy, South San Francisco, CA (US)

(21) Appl. No.: 17/441,572

(22) PCT Filed: Mar. 20, 2020
(86) PCT No.: PCT/US2020/023994

§ 371 Date: Sep. 21, 2021

Related U.S. Application Data
(60) Provisional application No. 62/821,826, filed on Mar. 21, 2019.

(51) Int. Cl. C07D 239/34; C07C 279/28; C07C 279/24; C07C 279/12; C07D 213/68; C07D 213/82; C07D 261/12

(52) U.S. Cl. CPC C07D 239/34; C07C 279/28; C07C 279/24; C07B 2200/07; C07D 213/68; C07D 213/82; C07D 261/12; C07C 279/12

(57) ABSTRACT

Therapeutics targeting the bacterium Porphyromonas gingivalis, including its proteases arginine gingipain A and arginine gingipain B, are disclosed, as well as the use thereof for the treatment of disorders associated with P. gingivalis infection, including brain disorders such as Alzheimer's disease. In certain embodiments, the invention provides compounds according to Formula I, Formula Ia, and Formula Ib, as described herein, and pharmaceutically acceptable salts thereof.

### CROSS-REFERENCES TO RELATED APPLICATIONS

The present application claims priority to U.S. Provisional Pat. Appl. No. 62/821,926, filed on Mar. 21, 2019, which application is incorporated herein by reference in its entirety.

### BACKGROUND OF THE INVENTION

Infection with the bacterium Porphyromonas gingivalis has been linked to the development of periodontal disease, Alzheimer's disease and other brain disorders, cardiovascular disease, diabetes, cancer, liver disease, kidney disease, preterm birth, arthritis, pneumonia and other disorders. P. gingivalis is an anaerobic asaccharolytic gram-negative rod bacterium that is known to infect the oral cavity and translocate systemically into coronary arteries, aorta, placental tissue, the brain, the kidneys, and the liver. The bacterium has also been identified in cancerous tissues and a mechanism has been proposed by which gingipains can trigger immortalization and metastasis. See: Gandhimadhi, et al. Journal of Indian Society of Periodontology. 2010; 14(2):114-120; Liao, et al., Med Hypotheses, 2009. 72(6): 732-5; Byrne, et al., Oral Microbiol Immunol, 2009. 24(6): 469-77; Mahendra, et al., J Maxillofac Oral Surg, 2009. 8(2): 108-13; Stelzel, et al., J Periodontol, 2002. 73(8): 868-70; Katz, et al., Journal of Dental Research, 2009. 88(6): 575-578; Poole, et al., J Alzheimers Dis, 2015, 43(1): 67-80; Ishikawa, et al., Biochim Biophys Acta, 2013. 1832(12): 2035-2043; Inaba, et al., Cellular Microbiology, 2014. 16(1): 131-145.

P. gingivalis produces proteases called gingipains, including Arginine Gingipain A (RgpA), Arginine Gingipain B (RgpB) and Lysine Gingipain (Kgp). Gingipains contribute to many functions of the organism including its survival and virulence. Gingipains can be secreted, transported to outer membrane surfaces of P. gingivalis, or released in outer membrane vesicles by the bacterium. Gingipains degrade a broad range of proteins (e.g., immunoglobulins, proteinase inhibitors, actin, and collagen) which can lead to cytoskeleton collapse and apoptosis in many types of cells. Recent research has demonstrated that inhibitors of gingipains can prevent P. gingivalis-induced cell death. See: Travis, et al., Adv Exp Med Biol, 2000. 477: 455-65; Sheets, et al., Infect Immun, 2005. 73(3): 1543-52; Sheets, et al., Infect Immun, 2006. 74(10): 5667-78; Stathopoulou, et al., BMC Microbiol, 2009. 9: 107.

### BRIEF SUMMARY OF THE INVENTION

Provided herein are compounds according to Formula I:

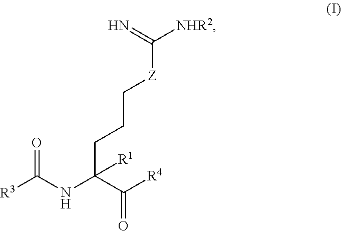

and pharmaceutically acceptable salts thereof, wherein:

Z is selected from the group consisting of NH, CH₂, and S;

W is selected from the group consisting of H and C₁₋₄ alkyl;

R² is selected from the group consisting of H, —CN, —OH, —OR^{2a}, —C(O)R^{2a}, and —C(O)OR^{2a};

R^{2a} is selected from the C₁₋₈ alkyl, C₆₋₁₀ aryl, and C₇₋₁₈ arylalkyl;

R³ is selected from the group consisting of C₃₋₈ cycloalkyl, C₃₋₈ alkyl, 3- to 12-membered heterocyclyl, C₆₋₁₀ aryl, and 5- to 12-membered heteroaryl, wherein R³ is optionally substituted with one or more R^{3a} substituents;

each R^{3a} is independently selected from the group consisting of halogen, —CN, —NO₂, —N₃, —OH, C₁₋₄ alkyl, C₁₋₄ haloalkyl, C₁₋₄ alkoxy, C₁₋₄ haloalkoxy, —N(R^{c})₂, —N⁺ (R^{b})₃, —(CH₂)ₖC(O)R^{b}, —NR^{c}(CH₂)ᵤC(O)R^{b}, —O(CH₂)ᵤC(O)R^{b}, —(CH₂)ₖCONR^{c}R^{c}, —(CH₂)ₖNR^{c}C(O)R^{b}, —NR^{c}(CH₂)ᵤCONR^{c}R^{c}, —NR^{c}(CH₂)ᵤNR^{c}C—(O)R^{b}, —O(CH₂)ᵤCONR^{c}R^{c}, and —O(CH₂)ᵤNR^{c}C(O)R^{b}, and optionally substituted triazolyl;

each R^{b} is independently selected from the group consisting of C₁₋₄ alkyl, C₁₋₄ haloalkyl, and C₁₋₄ deuteroalkyl;

each R^{c} is independently selected from the group consisting of H and C₁₋₈ alkyl;

each subscript k is independently selected from 0, 1, 2, 3, 4, 5, and 6;

each subscript u is independently selected from 1, 2, 3, 4, 5, and 6;

R⁴ is selected from the group consisting of —CH₂R^{4a} and —CHS(O)(R^{4b})₂;

R^{4a} is selected from the group consisting of —O—R⁵, —SO—R⁶, 3- to 12-membered heterocyclyl, and 5- to 12-membered heteroaryl,
wherein 3- to 12-membered heterocyclyl is optionally substituted with one or more members independently selected from the group consisting of oxo, halogen, C1-4 alkyl, and C1-4 haloalkyl, and
5- to 12-membered heteroaryl is optionally substituted with one or more members independently selected from the group consisting of halogen, C₁₋₄ alkyl, and C₁₋₄ haloalkyl;

each R^{4b} is independently selected C₁₋₈ alkyl; and

R⁵ and R⁶ are selected from the group consisting of phenyl, C₁₋₈ alkyl, C₁₋₈ haloalkyl, and 5- to 12-membered heteroaryl,
wherein phenyl is optionally substituted with 1-5 halogens, and
wherein 5- to 12-membered heteroaryl is optionally substituted with one or more halogen, C₁₋₄ alkyl, or C₁₋₄ haloalkyl;

provided that R⁴ is other than 2,3,5,6-tetrafluorophenoxymethyl.

Also provided herein are pharmaceutical compositions containing compounds of the disclosure, or a pharmaceutically acceptable salts thereof, and one or more pharmaceutically acceptable excipients.

Also provided are methods of inhibiting a gingipain. The methods include contacting the gingipain with an effective amount of a compound as described herein.

Also provided are methods of treating a disease or condition associated with P. gingivalis infection. The methods include administering to a subject in need thereof an effective amount of a compound or pharmaceutical composition as described herein.

### DETAILED DESCRIPTION OF THE INVENTION

The present invention provides potent nonpeptidic compounds for inhibition of arginine gingipains. The compounds can be used to prevent cell death, inflammation, and other pathology in a variety of diseases associated with P. gingivalis infection, including aging-related conditions such as Alzheimer's disease.

### I. DEFINITIONS

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. For example, “substituted alkyl” groups can be substituted with one or more groups selected from halo, hydroxy, amino, alkylamino, amido, acyl, nitro, cyano, and alkoxy.

As used herein, the term “alkoxy,” by itself or as part of another substituent, refers to a group having the formula —OR, wherein R is alkyl.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. For example, “substituted cycloalkyl” groups can be substituted with one or more groups selected from halo, hydroxy, amino, alkylamino, amido, acyl, nitro, cyano, and alkoxy.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group.

As used herein, the term “heteroalkyl,” by itself or as part of another substituent, refers to an alkyl group of any suitable length and having from 1 to 3 heteroatoms such as N, O and S. For example, heteroalkyl can include ethers, thioethers and alkyl-amines.

Additional heteroatoms can also be useful, including, but not limited to, B, Al, Si and P. The heteroatoms can be oxidized to form moieties such as, but not limited to, —S(O)— and —S(O)₂—. The heteroatom portion of the heteroalkyl can replace a hydrogen of the alkyl group to form a hydroxy, thio, or amino group. Alternatively, the heteroatom portion can be the connecting atom, or be inserted between two carbon atoms.

As used herein, the terms “halo” and “halogen,” by themselves or as part of another substituent, refer to a fluorine, chlorine, bromine, or iodine atom.

As used herein, the term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₈. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

As used herein, the term “haloalkoxy,” by itself or as part of another substituent, refers to an alkoxy group where some or all of the hydrogen atoms are replaced with halogen atoms.

As used herein, the term “halocycloalkyl,” by itself or as part of another substituent, refers to a cycloalkyl group where some or all of the hydrogen atoms are replaced with halogen atoms.

As used herein, the term “deuteroalkyl,” by itself or as part of another substituent, refers to an alkyl group where some or all of the hydrogen atoms are replaced with deuterium atoms. As for alkyl groups, deuteroalkyl groups can have any suitable number of carbon atoms, such as C₁₋₈. In some instances, the term “perdeutero” can be used to define a compound or radical where all the hydrogens are replaced with deuterium.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. For example, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. For example, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

As used herein, the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. For example, “substituted heterocyclyl” groups can be substituted with one or more groups selected from halo, hydroxy, amino, oxo, alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₂, wherein each R group is H or alkyl. An amino moiety can be ionized to form the corresponding ammonium cation.

As used herein, the term “sulfonyl” refers to a moiety —SO₂R, wherein the R group is alkyl, haloalkyl, or aryl. An amino moiety can be ionized to form the corresponding ammonium cation. “Alkylsulfonyl” refers to an amino moiety wherein the R group is alkyl.

As used herein, the term “hydroxy” refers to the moiety —OH.

As used herein, the term “cyano” refers to a carbon atom triple-bonded to a nitrogen atom (i.e., the moiety —C≡N).

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents are generally those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In general, “substituted,” as used herein, does not encompass replacement and/or alteration of a key functional group by which a molecule is identified, e.g., such that the “substituted” functional group becomes, through substitution, a different functional group. For example, a “substituted phenyl” group must still comprise the phenyl moiety and cannot be modified by substitution, in this definition, to become, e.g., a cyclohexyl group.

Examples of suitable monovalent substituents on a substitutable carbon atom of an “optionally substituted” group are independently halogen; —(CH₂)₀₋₄R^{α}; —(CH₂)₀₋₄OR^{α}; —O(CH₂)₀₋₄R^{α}, —O—(CH₂)₀₋₄C(O)OR^{α}; —(CH₂)₀₋₄CH(OR^{α})₂; —(CH₂)₀₋₄SR^{α}; —(CH₂)₀₋₄Ph, wherein Ph is phenyl which may be substituted with R^{α}; —(CH₂)₀₋₄O(CH₂)₀₋₁ phenyl, which phenyl may be substituted with R^{α}; —CH═CHPh, wherein Ph is phenyl which may be substituted with R^{α}; —(CH₂)₀₋₄O(CH₂)₀₋₁-Py, wherein Py is pyridyl which may be substituted with R^{α}; —NO₂; —CN; —N₃; —(CH₂)₀₋₄N(R^{α})₂; —(CH₂)₀₋₄N(R^{α})C(O)R^{α}; —N(R^{α})C(S)R^{α}; —(CH₂)_{0- 4}N(R^{α})C(O)NR^{α}₂; —N(R^{α})C(S)NR^{α}₂; —(CH₂)₀₋₄N(R^{α})C(O)OR^{α}; —N(R^{α})N(R^{α})C(O)R^{α}; —N(R^{α})N(R^{α})C(O)NR^{α}₂; —N(R^{α})N(R^{α})C(O)OR^{α}; —(CH₂)₀₋₄C(O)R^{α}; —C(S)R^{α}; —(CH₂)₀₋₄C(O)OR^{α}; —(CH₂)₀₋₄C(O)SR^{α}; —(CH₂)₀₋₄C(O)OSiR^{α}₃; —(CH₂)₀₋₄OC(O)R^{α}; —OC(O)(CH₂)₀₋₄SR—SC(S)SR^{α}; —(CH₂)₀₋₄SC(O)R^{α}; —(CH₂)₀₋₄C(O)NR^{α}₂; —C(S)NR^{α}₂, ——C(S)SR^{α}; —SC(S)SR^{α}, —(CH₂)₀₋₄OC(O)NR^{α}₂; —C(O)N(OR^{α})R^{α}; —C(O)C(O)R^{α}; —C(O)CH₂C(O)R^{α}; —C(NOR^{α})R^{α}; —(CH₂)₀₋₄SSR^{α}; —(CH₂)₀₋₄S(O)₂R^{α}; —(CH₂)₀₋₄S(O)₂OR^{α}; —(CH₂)₀₋₄OS(O)₂R^{α}; —S(O)₂NR^{α}₂; —(CH₂)₀₋₄S(O)R^{α}; —N(R^{α})S(O)₂NR^{α}₂; —N(R^{α})S(O)₂R^{α}; —N(OR^{α})R^{α}; —C(NH)NR^{α}₂; —P(O)₂R^{α}; —P(O)R^{α}₂; —OP(O)R^{α}₂; —OP(O)(OR^{α})₂; —SiR^{α}₃; —(C₁₋₄ straight or branched)alkylene)-O—N(R^{α})₂; or —(C₁₋₄ straight or branched)alkylene)-C(O)O—N(R^{α})₂. Each R^{α} is independently hydrogen; C₁₋₈ alkyl; —CH₂Ph, —O(CH₂)₀₋₁Ph; —CH₂-(5- to 6-membered heteroaryl); C₃₋₈ cycloalkyl; C₆₋₁₀ aryl; 4- to 10-membered heterocyclyl; or 6- to 10-membered heteroaryl; and each R^{α} may be further substituted as described below.

Examples of suitable monovalent substituents on R^{α} are independently halogen, —(CH₂)₀₋₂R^{β}; —(CH₂)₀₋₂OH; —(CH₂)₀₋₂OR^{β}; —(CH₂)₀₋₂CH(OR^{β})₂; —CN; —N₃; —(CH₂)₀₋₂C(O)R^{β}; —(CH₂)₀₋₂C(O)OH; —(CH₂)₀₋₂C(O)OR^{β}; —(CH₂)₀₋₂SR^{β}; —(CH₂)₀₋₂SH; —(CH₂)₀₋₂NH₂, —(CH₂)₀₋₂NHR^{β}; —(CH₂)₀₋₂NR^{β}₂; —NO₂; Si^{β}₃; —OSiR^{β}₃; —C(O)SR^{β}; —(C₁₋₄ straight or branched alkylene)C(O)OR^{β}; or —SSR^{β}; wherein each R^{β} is independently selected from C₁₋₄ alkyl; —CH₂Ph; —O(CH₂)₀₋₁Ph; C₃₋₈ cycloalkyl; C₆₋₁₀ aryl; 4- to 10-membered heterocyclyl; or 6- to 10-membered heteroaryl. Suitable divalent substituents on a saturated carbon atom of R^{α} include ═O and ═S.

Examples of suitable divalent substituents on a saturated carbon atom of an “optionally substituted” group include the following: ═O; ═S; ═NNR^{γ}₂; ═NNHC(O)R^{γ}; ═NNHC(O)OR^{γ}; ═NNHS(O)₂R^{γ}; ═NR^{γ}; ═NOR^{γ}; —O(C(R^{γ}₂))₂₋₃O—; or —S(C(R^{γ}₂))₂₋₃S—; wherein each independent occurrence of RY is selected from hydrogen; C₁₋₈ alkyl, which may be substituted as defined below; C₃₋₈ cycloalkyl; C₆₋₁₀ aryl; 4- to 10-membered heterocyclyl; or 6- to 10-membered heteroaryl. Suitable divalent substituents that are bound to vicinal substitutable carbons of an “optionally substituted” group include: —O(CR^{β}₂)₂₋₃O—; wherein each independent occurrence of R^{β} is selected from hydrogen; C₁₋₈ alkyl which may be substituted as defined below; C₃₋₈ cycloalkyl; C₆₋₁₀ aryl; 4- to 10-membered heterocyclyl; or 6- to 10-membered heteroaryl.

Examples of suitable substituents on the alkyl group of R^{γ} include halogen; —R^{δ}; —OH; —OR^{δ}; —CN; —C(O)OH; —C(O)OR^{δ}; —NH₂; —NHR^{δ}; —NR^{δ}₂; or —NO₂; wherein each R^{δ} is independently C₁₋₄ alkyl; —CH₂Ph; —O(CH₂)₀₋₁Ph; 4- to 10-membered heterocyclyl; or 6- to 10-membered heteroaryl.

Examples of suitable substituents on a substitutable nitrogen of an “optionally substituted” group include —R^{ε}; —NR^{ε}₂; —C(O)R^{ε}; —C(O)OR^{ε}; —C(O)C(O)R^{ε}; —C(O)CH₂C(O)R^{ε}; —S(O)₂R^{ε}; —S(O)₂NR^{ε}₂; —C(S)NR^{ε}₂; —C(NH)NR^{ε}₂; or —N(R^{ε})S(O)₂R^{ε}; wherein each R^{ε} is independently hydrogen; C₁₋₈ alkyl which may be substituted as defined below; C₃₋₈ cycloalkyl; C₆₋₁₀ aryl; 4- to 10-membered heterocyclyl; or 6- to 10-membered heteroaryl.

Examples of suitable substituents on the alkyl group of R^{ε} are independently halogen; —R^{δ}; —OH; —OR^{δ}; —CN; —C(O)OH; —C(O)OR^{δ}; —NH₂; —NHR^{δ}; —NR^{δ}₂; or —NO₂; wherein each R^{δ} is independently C₁₋₄ alkyl; —CH₂Ph; —O(CH₂)₀₋₁Ph; C₆₋₁₀ aryl; 4- to 10-membered heterocyclyl; or 6- to 10-membered heteroaryl.

As used herein, the term “pharmaceutically acceptable excipient” refers to a substance that aids the administration of an active agent to a subject. By “pharmaceutically acceptable,” it is meant that the excipient is compatible with the other ingredients of the formulation and is not deleterious to the recipient thereof. Useful pharmaceutical excipients include, but are not limited to, binders, fillers, disintegrants, lubricants, glidants, coatings, sweeteners, flavors and colors.

As used herein, the term “salt” refers to acid or base salts of the compounds of the disclosure. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic.

Pharmaceutically acceptable salts of the acidic compounds described herein are salts formed with bases, namely cationic salts such as alkali and alkaline earth metal salts (such as sodium, lithium, potassium, calcium, and magnesium salts), as well as ammonium salts (such as ammonium, trimethyl-ammonium, diethylammonium, and tris-(hydroxymethyl)-methyl-ammonium salts).

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, compounds which are in a prodrug form are provided. Prodrugs of the compounds described herein are those compounds that readily undergo chemical changes under physiological conditions to provide the parent compounds. Additionally, prodrugs can be converted to the parent compounds by chemical or biochemical methods in an ex vivo environment. For example, prodrugs can be slowly converted to the parent compounds when placed in a transdermal patch reservoir with a suitable enzyme or chemical reagent.

As used herein, the terms “Porphyromonas gingivalis” and “P. gingivalis” refer to the gram-negative asaccharolytic bacterium that is recognized as a key causative microbe in the pathogenesis of periodontitis and related conditions. “P. gingivalis infection” refers to the invasion and colonization of P. gingivalis in a bodily tissue such as the gums or the brain. P. gingivalis infection is frequently characterized by subsequent tissue injury and disease.

As used herein, the term “gingipain” refers to cysteine proteases expressed by P. gingivalis having trypsin-like specificity (i.e., Lys-Xaa and Arg-Xaa). Gingipains are recognized as the major virulence factors of P. gingivalis and contribute to bacterial attachment and colonization, nutrient acquisition, evasion of host defenses, and tissue invasion. The terms “arginine gingipain” and “Rgp” are used interchangeably to refer to the P. gingivalis arginine-specific gingipains RgpA and RgpB, classified under EC number EC 3.4.22.37. The rgpA and rgpB gene-translation products, RgpA and RgpB, share a caspase-like protease domain (specific for Arg-Xaa peptide bonds) and an immunoglobulin-like domain. In RgpA, the protease and immunoglobulin-like domains are followed by a large C-terminal extension containing hemagglutinin-adhesin domains.

As used herein, the term “inhibiting” refers to reducing the level of activity (e.g., proteolytic activity) of an enzyme such as a gingipain which can be assessed, for example, using an in vitro assay or other suitable assay. Inhibition of enzyme activity caused by a particular substance (e.g., a gingipain inhibitor as described herein) can be expressed as the percentage of the enzyme activity measured in the absence of the substance under similar conditions. The ability of a particular substance to inhibit an enzyme can be expressed as an IC₅₀ value, i.e., the concentration of the compound required to reduce the activity of the enzyme to 50% of its maximum activity.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, e.g., the result of a physical examination.

As used herein, the terms “effective amount” and “therapeutically effective amount” refer to a dose of a compound such as an Rgp inhibitor that inhibits the activity of a gingipain and/or produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

As used herein, the term “Alzheimer's disease” refers to a progressive disease of the central nervous system in humans and other mammals. It is manifested by dementia (especially in the elderly); disorientation; loss of memory; difficulty with language, calculation, or visual-spatial skills; and psychiatric manifestations. Alzheimer's disease is associated with progressive neurodegeneration and characteristic pathology, namely beta amyloid plaques and tau tangles.

As used herein, the term “subject” refers to animals such as mammals, including, but not limited to, primates (e.g., humans), cows, sheep, goats, horses, dogs, cats, rabbits, rats, mice and the like.

### II. GINGIPAIN INHIBITORS

Provided herein are compounds according to Formula I:

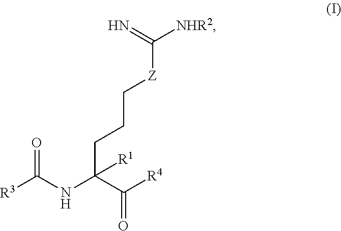

and pharmaceutically acceptable salts thereof, wherein:

Z is selected from the group consisting of NH, CH₂, and S;

R¹ is selected from the group consisting of H and C₁₋₄ alkyl;

R² is selected from the group consisting of H, —CN, —OH, —OR^{2a}, —C(O)R^{2a}, and —C(O)OR^{2a};

R^{2a} is selected from the C₁₋₈ alkyl, C₆₋₁₀ aryl, and C₇₋₁₈ arylalkyl;

R³ is selected from the group consisting of C₃₋₈ cycloalkyl, C₃₋₈ alkyl, 3- to 12-membered heterocyclyl, C₆₋₁₀ aryl, and 5- to 12-membered heteroaryl, wherein R³ is optionally substituted with one or more R^{3a} substituents;

each R^{3a} is independently selected from the group consisting of halogen, —CN, —NO₂, —N₃, —OH, C₁₋₄ alkyl, C₁₋₄ haloalkyl, C₁₋₄ alkoxy, C₁₋₄ haloalkoxy, —N(R^{c})₂, —N⁺(R^{b})₃, —(CH₂)ₖC(O)R^{b}, —NR^{c}(CH₂)ᵤC(O)R^{b}, —O(CH₂)ᵤC(O)R^{b}, —(CH₂)ₖCONR^{c}R^{c}, —(CH₂)ₖNR^{c}C(O)R^{b}, —NR^{c}(CH₂)ᵤCONR^{c}R^{c}, —NR^{c}(CH₂)ᵤNR^{c}C—(O)R^{b}, —O(CH₂)ᵤCONR^{c}R^{c}, and —O(CH₂)ᵤNR^{c}C(O)R^{b}, and optionally substituted triazolyl;

each R^{b} is independently selected from the group consisting of C₁₋₄ alkyl, C₁₋₄ haloalkyl, and C₁₋₄ deuteroalkyl;

each R^{c} is independently selected from the group consisting of H and C₁₋₈ alkyl;

each subscript k is independently selected from 0, 1, 2, 3, 4, 5, and 6;

each subscript u is independently selected from 1, 2, 3, 4, 5, and 6;

R⁴ is selected from the group consisting of —CH₂R^{4a} and —CHS(O)(R^{4b})₂;

R^{4a} is selected from the group consisting of —O—R⁵, —SO—R⁶, 3- to 12-membered heterocyclyl, and 5- to 12-membered heteroaryl,
wherein 3- to 12-membered heterocyclyl is optionally substituted with one or more members independently selected from the group consisting of oxo, halogen, C₁₋₄ alkyl, and C₁₋₄ haloalkyl, and
5- to 12-membered heteroaryl is optionally substituted with one or more members independently selected from the group consisting of halogen, C₁₋₄ alkyl, and C₁₋₄ haloalkyl;

each R^{4b} is independently selected C₁₋₈ alkyl; and

R⁵ and R⁶ are selected from the group consisting of phenyl, C₁₋₈ alkyl, C₁₋₈ haloalkyl, and 5- to 12-membered heteroaryl,
wherein phenyl is optionally substituted with 1-5 halogens, and
wherein 5- to 12-membered heteroaryl is optionally substituted with one or more halogen, C₁₋₄ alkyl, or C₁₋₄ haloalkyl;

provided that R⁴ is other than 2,3,5,6-tetrafluorophenoxymethyl.

In some embodiments, R² is H. In some embodiments, R² is —CN. In some embodiments, R² is selected from —OR^{2a}, —C(O)R^{2a}, and —C(O)OR^{2a}. In some embodiments, R^{2a} is arylalkyl (e.g., benzyloxy), which is optionally substituted with one or more substituents selected from halogen, —CN, —NO₂, —N₃, —OH, C₁₋₄ alkyl, C₁₋₄ haloalkyl, C₁₋₄ alkoxy, C₁₋₄ haloalkoxy, and —C(O)R^{2b}, wherein R^{2b} is C₁₋₈ alkyl or C₆₋₁₀ aryl. In some embodiments, R² is substituted or unsubstituted benzyloxycarbonyl (e.g., (4-benzoyl)benzyl-oxycarbonyl). In some embodiments, compounds wherein R² is CN, —OH, —OR^{2a}, —C(O)R^{2a}, or —C(O)OR^{2a} can function as prodrugs for conversion to the corresponding compounds wherein R² is H (e.g., upon administration to a subject).

Some embodiments of the present disclosure provide compounds having a structure according to Formula Ia:

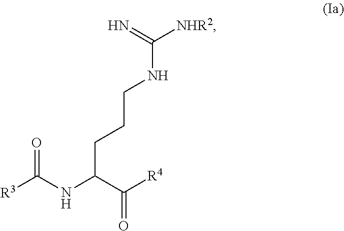

and pharmaceutically acceptable salt thereof.

Some embodiments of the present disclosure provide compounds having a structure according to Formula Ib:

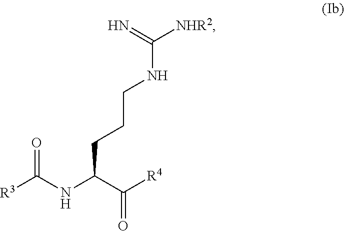

and pharmaceutically acceptable salt thereof.

In some embodiments, R³ in compounds of Formula I, Formula Ia, or Formula Ib is selected from C₃₋₈ cycloalkyl, C₃₋₈ alkyl, C₆₋₁₀ aryl, 5- to 12-membered heteroaryl, and 3- to 12-membered heterocyclyl, each of which is optionally substituted with one or more R^{3a} substituents. For example, R³ can be cyclopropyl, cyclobutyl, cyclopentyl, cyclohexyl, cycloheptyl, or cyclooctyl. R³ can be n-propyl, isopropyl, n-butyl, isobutyl, sec-butyl, tert-butyl, n-pentyl, branched pentyl, n-hexyl, branched hexyl, n-heptyl, branched heptyl, n-octyl, or branched octyl.

In some embodiments, R³ is C₃₋₈ alkyl, which is optionally substituted with one or more R^{3a}. In some embodiments, R³ is selected from the group consisting of C₃₋₈ cycloalkyl and 5- to 12-membered heteroaryl. In some embodiments, R³ is selected from unsubstituted or substituted cyclobutyl, unsubstituted or substituted cyclopentyl, and unsubstituted or substituted cyclohexyl. In some embodiments, R³ is unsubstituted or substituted isopropyl.

In some embodiments, R³ is selected from unsubstituted or substituted phenyl and unsubstituted or substituted naphthyl. In some embodiments, R³ is selected from unsubstituted or substituted pyrrolyl, unsubstituted or substituted pyridinyl, unsubstituted or substituted imidazolyl, unsubstituted or substituted pyrazolyl, unsubstituted or substituted triazolyl, unsubstituted or substituted pyrazinyl, unsubstituted or substituted triazinyl, unsubstituted or substituted indolyl, unsubstituted or substituted isoindolyl, and unsubstituted or substituted quinolinyl.

In some embodiments, R³ is selected from cyclopentyl and phenyl, each of which is optionally substituted with one or more R^{3a} substituents. In some such embodiments, each R^{3a} is independently selected from halogen, —N₃, C₁₋₄ alkyl, C₁₋₄ haloalkyl, C₁₋₄ alkoxy, C₁₋₄ haloalkoxy, and —NR^{c}C(O)R^{b}. In some embodiments, R³ is cyclopentyl.

In some embodiments, R³ is selected from isopropyl, cyclopentyl, phenyl, pyridin-2-yl, pyridin-3-yl, and pyridin-4-yl, each of which is optionally substituted with one or more R^{3a} substituents. In some such embodiments, each R^{3a} is independently selected from halogen, —N₃, C₁₋₄ alkyl, C₁₋₄ haloalkyl, C₁₋₄ alkoxy, C₁₋₄ haloalkoxy, C₁₋₄ alkoxy, C₁₋₄ haloalkoxy, —N(R^{c})₂, —N⁺(R^{b})₃, and —NR^{c}C(O)R^{b}.

In some embodiments, R³ and the carbonyl moiety to which it is attached form an amino acid residue. As used herein, the term “amino acid residue” refers to a moiety wherein the grouping R³C(O)— has the structure:

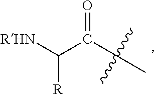

wherein R represents the side chain of a naturally occurring amino acid (e.g., an alanine sidechain, an arginine sidechain, an asparagine sidechain, an aspartic acid sidechain, a cysteine sidechain, a glutamine sidechain, a glutamic acid sidechain, a glycine sidechain, a histidine sidechain, an isoleucine sidechain, a leucine sidechain, a lysine sidechain, a methionine sidechain, a phenylalanine sidechain, a proline sidechain, a selenocysteine sidechain, a serine sidechain, a threonine sidechain, a tryptophan sidechain, a tyrosine sidechain, or a valine sidechain) or the side chain of a non-naturally occurring amino acid (e.g., an azidohomoalanine sidechain, a propargylglycine sidechain, a p-acetylphenylalanine sidechain, or the like); R′ is hydrogen, C₁₋₆ alkyl, C₂₋₆ acyl (e.g., acetyl), or an amine protecting group as described herein; and the wavy line represenst the point of connection from the amino acid residue to the the remainder of the molecule. In some embodiments, the grouping R³C(O)— forms an L- or D-alanine residue, an L- or D-arginine residue, an L- or D-asparagine residue, an L- or D-aspartic acid residue, an L- or D-cysteine residue, an L- or D-glutamine residue, an L- or D-glutamic acid residue, an L- or D-glycine residue, an L- or D-histidine residue, an L- or D-isoleucine residue, an L- or D-leucine residue, an L- or D-lysine residue, an L- or D-methionine residue, an L- or D-phenylalanine residue, an L- or D-proline residue, an L- or D-selenocysteine residue, an L- or D-serine residue, an L- or D-threonine residue, an L- or D-tryptophan residue, an L- or D-tyrosine residue, or an L- or D-valine residue, each of which optionally comprises an N-acetyl group (e.g., wherein R′ as depicted above is CH₃C(O)—).

In some embodiments, R⁴ in compounds of Formula I, Formula Ia, or Formula Ib is —CH₂OR⁵ and R⁵ is C₁₋₈ haloalkyl. In such embodiments, R⁵ can be, e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like. In some embodiments, R⁴ is selected from 2,2,2-trifluoroethoxy and 1,1,1,3,3,3-hexafluoroisopropoxy. In some such embodiments, R³ is selected from (2-methoxy)propan-2-yl, unsubstituted phenyl, phenyl substituted with one or more halogen, —N₃, C₁₋₄ haloalkoxy, and/or —NR^{c}C(O)R^{b}, unsubstituted pyridinyl, and pyridinyl substituted with one or more halogen, —N(R^{c})₂, and/or —N⁺(R^{b})₃.

In some embodiments, R⁴ in compounds of Formula I, Formula Ia, or Formula Ib is —CH₂OR⁵ and R⁵ is 3- to 12-membered heterocyclyl or 5- to 12-membered heteroaryl, each of which is optionally substituted with one or more halogen, C₁₋₄ alkyl, or C₁₋₄ haloalkyl. In some embodiments, heterocyclyl or heteroaryl is optionally substituted with one or more halogen, C₁₋₃ alkyl, or C₁₋₃ haloalkyl. R⁵ can be, for example, isoxazolyl, oxazolyl, imidazolyl, pyrazolyl, pyridinyl, oxazinyl, pyrimidinyl, pyrazinyl, pyridazinyl. In some embodiments, R⁴ is —O—R⁵ and R⁵ is selected from pyridin-2-yl, pyridin-3-yl, pyridin-4-yl, isoxazol-3-yl, isoxazol-4-yl, isoxazol-5-yl, pyrimidin-2-yl, pyrimidin-4-yl, pyrimidin-5-yl, and pyrimidin-6-yl. In some embodiments, R⁴ is —O—R⁵ and R⁵ is selected from isoxazol-3-yl, pyridin-3-yl, pyridin-4-yl, 2,6-dimethylpyridin-5-yl, and 2-methylpyrimidin-5-yl. In some embodiments, R⁴ is —O—R⁵, R⁵ is selected from isoxazol-3-yl, pyridin-3-yl, pyridin-4-yl, 2,6-dimethylpyridin-5-yl, and 2-methylpyrimidin-5-yl, and R³ is selected from (2-methoxy)propan-2-yl, unsubstituted phenyl, phenyl substituted with one or more halogen, —N₃, C₁₋₄ haloalkoxy, and/or —NR^{c}C(O)R^{b}, unsubstituted pyridinyl, and pyridinyl substituted with one or more halogen, —N(R^{c})₂, and/or —N⁺(R^{b})₃.

In some embodiments, R⁴ in compounds of Formula I, Formula Ia, or Formula Ib is —CH₂OR⁵ and R⁵ is phenyl, which is optionally substituted with 1-5 halogens. In some embodiments, R⁵ in compounds of Formula I, Formula Ia, or Formula Ib is selected from 2-fluorophenoxy; 3-fluorophenoxy; 4-fluorophenoxy; 2,3-difluorophenoxy; 2,4-difluoro-phenoxy; 2,5-difluorophenoxy; 2,6-difluorophenoxy; 3,4-difluorophenoxy; 3,5-difluoro-phenoxy; 2,3,6-trifluorophenoxy; and 2,3,5-trifluorophenoxy. In some such embodiments, R^{5a} is selected from 2-fluorophenoxy; 3 fluorophenoxy; 2,3-difluorophenoxy; 2,5-difluoro-phenoxy; 2,6-difluorophenoxy; 3,5-difluorophenoxy; 2,3,6-trifluorophenoxy; and 2,3,5-trifluorophenoxy. In some such embodiments, R⁵ is selected from 2,6-difluorophenoxy and 2,3,6-trifluorophenoxy. In some such embodiments, R³ is selected from (2-methoxy)propan-2-yl, unsubstituted phenyl, phenyl substituted with one or more halogen, —N₃, C₁₋₄ haloalkoxy, and/or —NR^{c}C(O)R^{b}, unsubstituted pyridinyl, and pyridinyl substituted with one or more halogen, —N(R^{c})₂, and/or —N⁺(R^{b})₃.

In some embodiments, the compound is selected from the group consisting of:

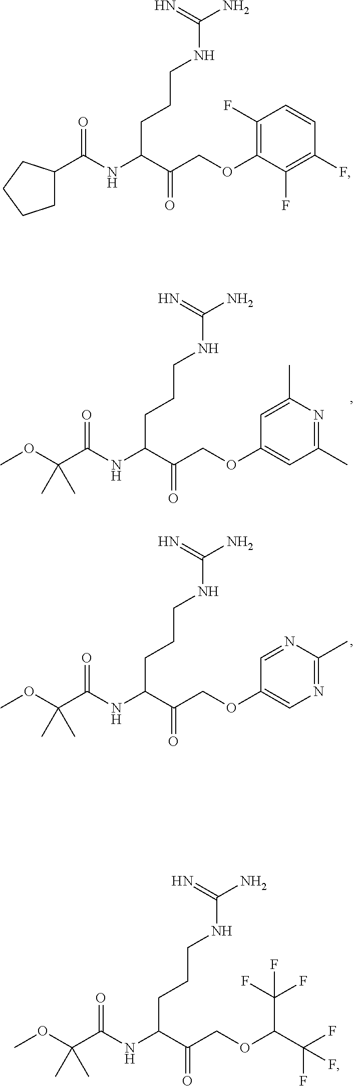

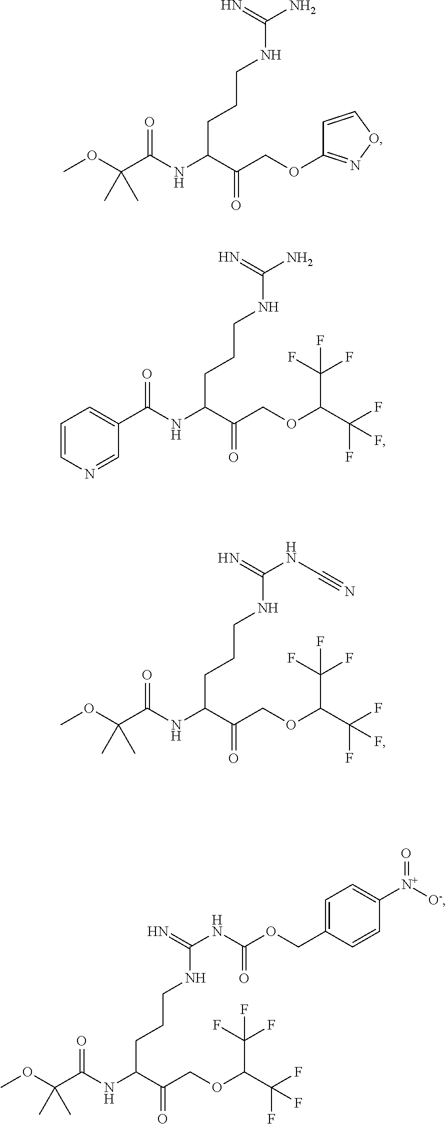

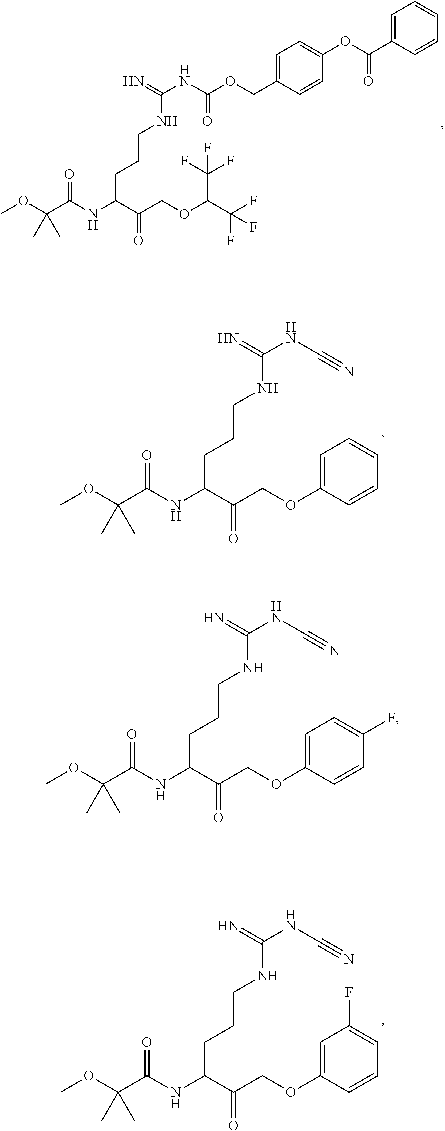

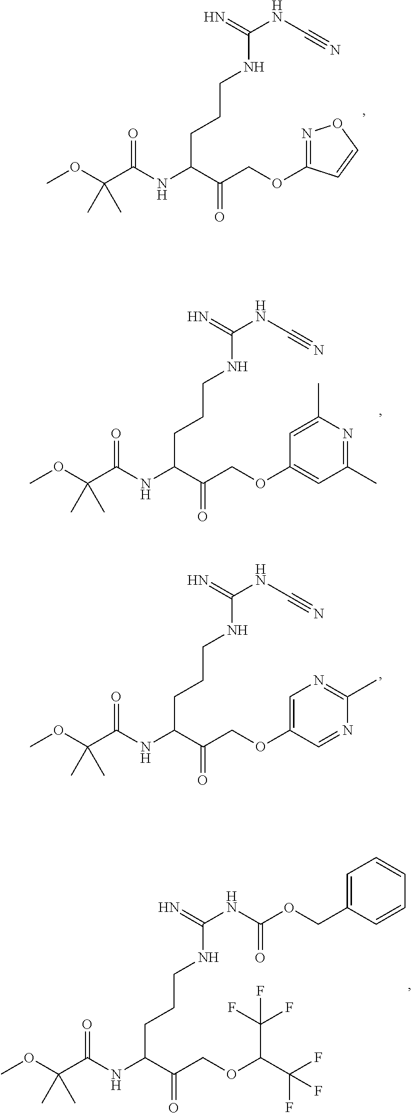

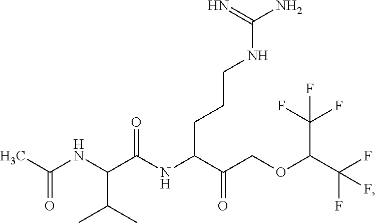

and pharmaceutically acceptable salts thereof.

In some embodiments, the compound is selected from the group consisting of:

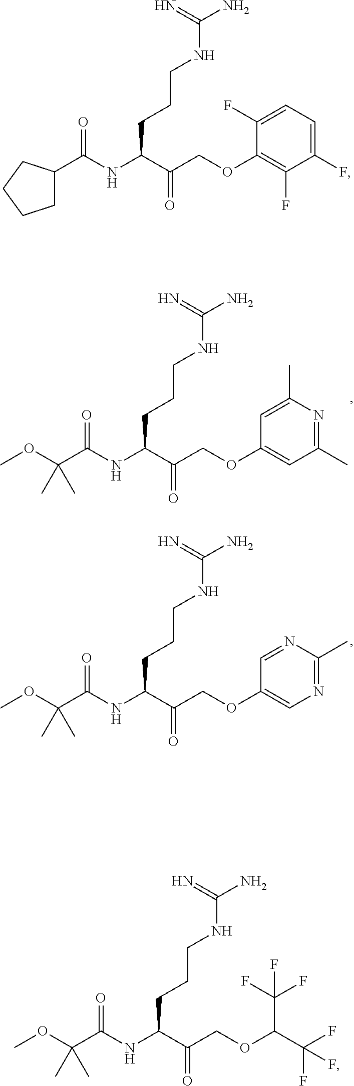

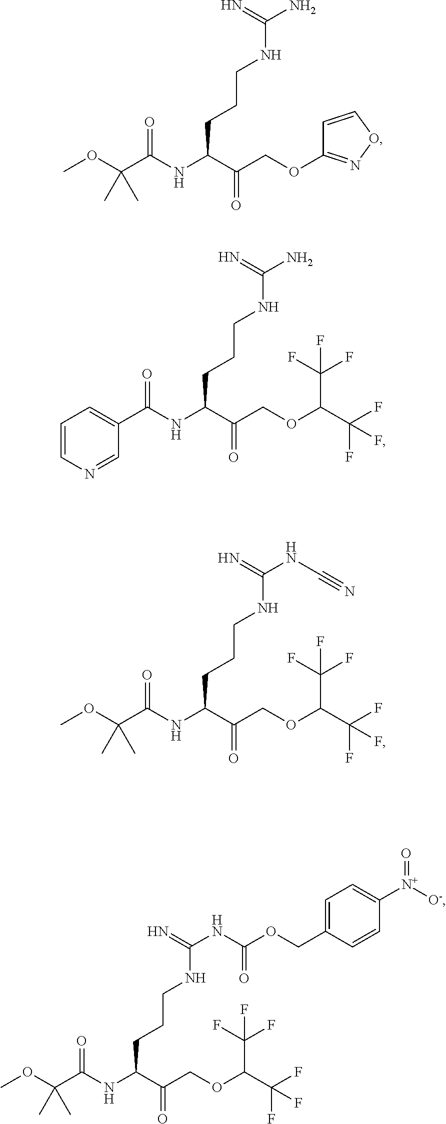

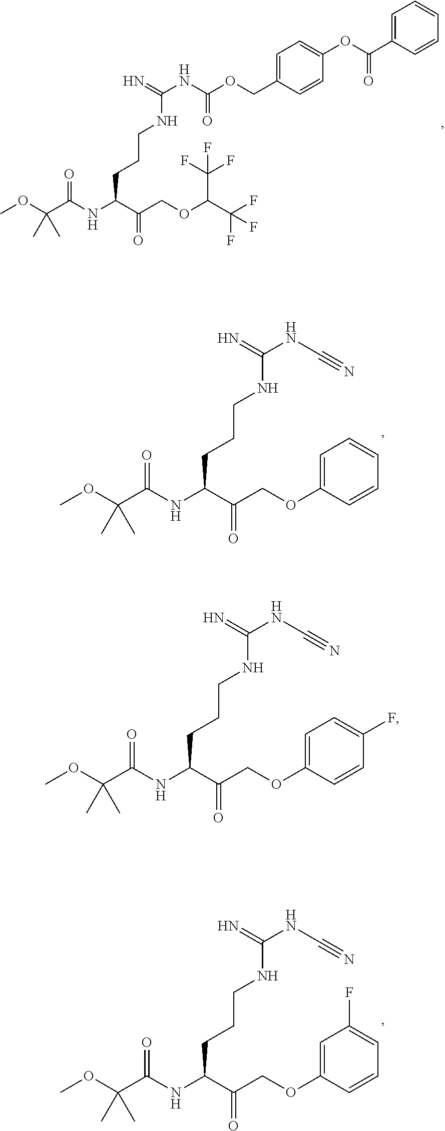

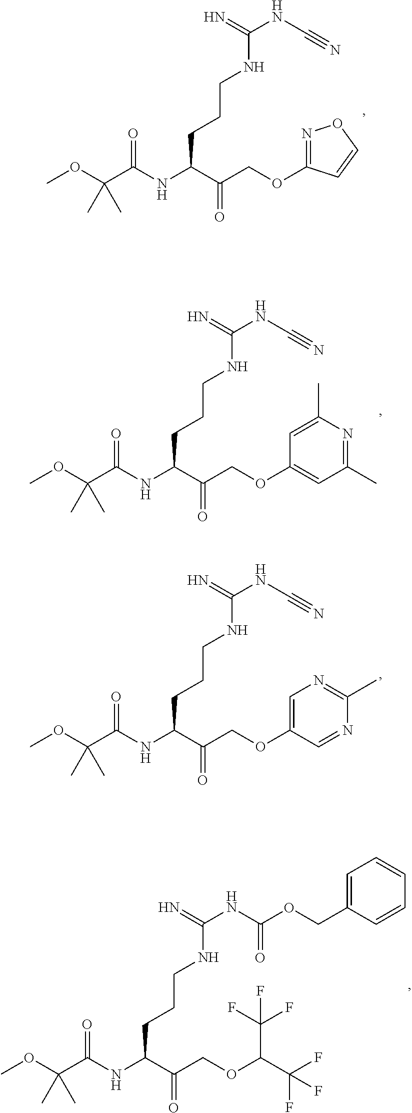

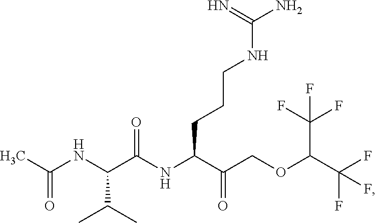

and pharmaceutically acceptable salts thereof.

Compounds according to the present disclosure may be prepared as summarized in the following schemes and described below. As shown in Scheme 1, protected ornithine starting material (i) can be treated with a carboxylic acid R³CO₂H, a racemization inhibitor (for example HOBt), and a dehydrating agent (for example EDAC), in an organic solvent (for example DMF), generating amide (ii). Alternatively, the protected ornithine can be treated with R³COX, wherein X is a leaving group (for example chloride), and an organic base (for example Et3N), in an organic solvent (for example CH₂Cl₂), generating amide (ii). A variety of applicable carboxylic acids (R³CO₂H) and derivatives thereof (R³COX) are commercially available, or can be prepared according to known methods. Amide (ii) may be converted to protected ketone (iii) by various routes. In one non-limiting example, the amide is hydrolyzed using a strong based such as NaOH. The resulting carboxylic acid is then reacted with ClCO₂Et, a tertiary amine, and diazo methane to form a diazomethyl ketone, which can then be treated with HBr to provide a bromomethyl ketone. In another sequence, the —OR moiety may be converted to a chloromethyl group in one step using ClCH₂I and LiN(iPr)₂. Bromomethyl ketones and chloromethyl ketones can be heated with substituted phenols and KF in DMF to provide aryloxymethyl ketones (iii). In another non-limiting example, the bromomethyl or chloromethyl ketones are treated with isoxazole-5-one and KF in DMF to provide isoxazolyloxymethyl ketones (iii). Ketone (iii) can be reduced (e.g., using sodium borohydride) to corresponding alcohol (v) prior to reaction with carbamimidothioate (vi) to yield guanidine intermediate (vii). Oxidation of guanidine intermediate (vii) (e.g., with Dess-Martin periodinane) and deprotection of resulting ketone (viii) yields product (ix). As an alternative to carbamimidothioate (vi), a dicyanoamine or salt thereof (e.g., dicyanoamino sodium) may be employed to provide intermediates and/or products wherein R² is cyano. Compounds wherein R² is H may be converted to compounds wherein R² is, for example, —C(O)R^{2a} or —C(O)OR^{2a}, via treatment with with a compound R^{2a}CO₂H or R^{2a}OCO₂H or activated derivative R^{2a}COX or R^{2a}OCOX in the manner described above.

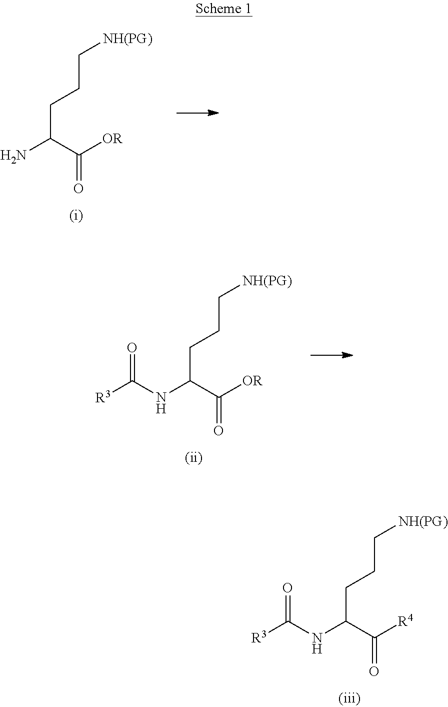

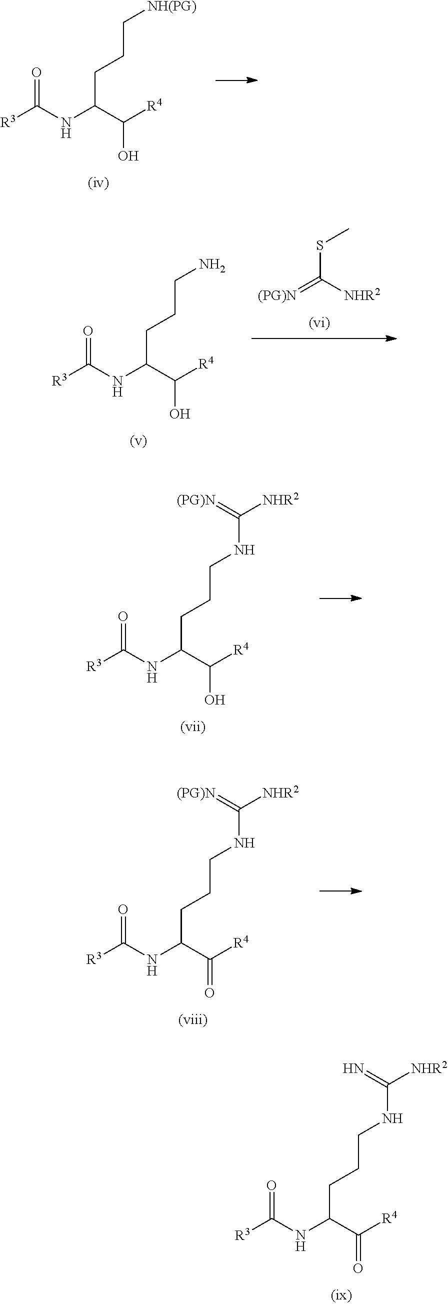

As used herein, the term “protecting group” refers to a chemical moiety that renders a functional group (e.g., an amino group) unreactive, but is also removable so as to restore the amino group. Examples of protecting groups include, but are not limited to, benzyloxy-carbonyl (Z or Cbz); 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. Other protecting groups are known to those of skill in the art including, for example, those described by Green and Wuts (Protective Groups in Organic Synthesis, 4^{th} Ed. 2007, Wiley-Interscience, New York).

As shown in Scheme 2, hydroxynorvaline starting material (xi) can acylated as described above to generate amide (xii) which can be converted to silyl ether (xiii). Silyl ether (xiii) can be esterified (e.g., using diazomethane) prior to conversion to protected ketones (xv) having various R⁴ groups using the routes described in conjunction with Scheme 1. Ketone (xv) can be reduced to the corresponding alcohol (xvi) and the silyl ether can be removed for conversion of the alcohol to a halide leaving group (e.g., using N-bromosuccinimide and triphenylphosphine) for displacement with thiourea (xviii). The resulting amidine intermediate can be reoxidized to provide product (ix).

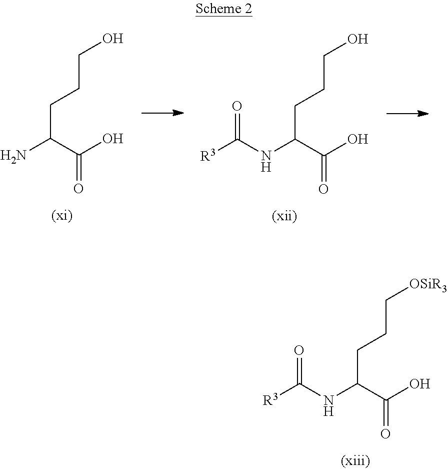

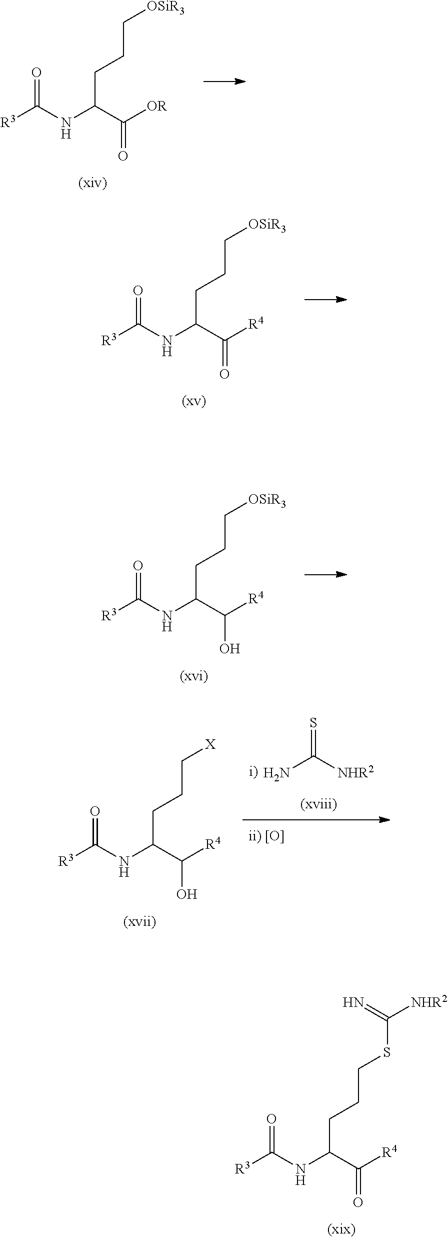

As shown in Scheme 3, hydroxynorleucine starting material (xxi) can be converted to nitrile (xxii), e.g., by converting the alcohol to an iodide under Appel conditions with iodine, triphenylphosphine, and imidazole prior to reaction with potassium cyanide. Nitrile (xxii) can then be deprotected and acylated to generate amide (xxiii) which can be converted to protected ketones (xxiv) having various R⁴ groups, as described above. Ketone (xxiv) can be reduced to the corresponding alcohol (xxv) prior to displacement with hydroxylamine R²NHOH and hydrogenation. The resulting amidine intermediate can be reoxidized to provide product (xxvi).

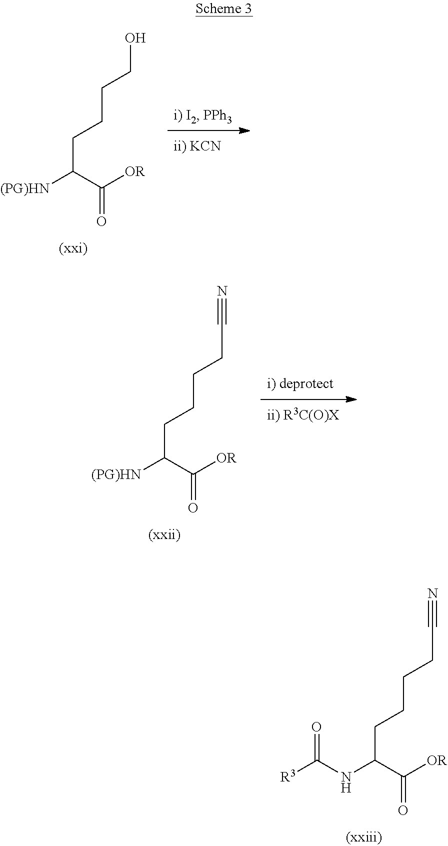

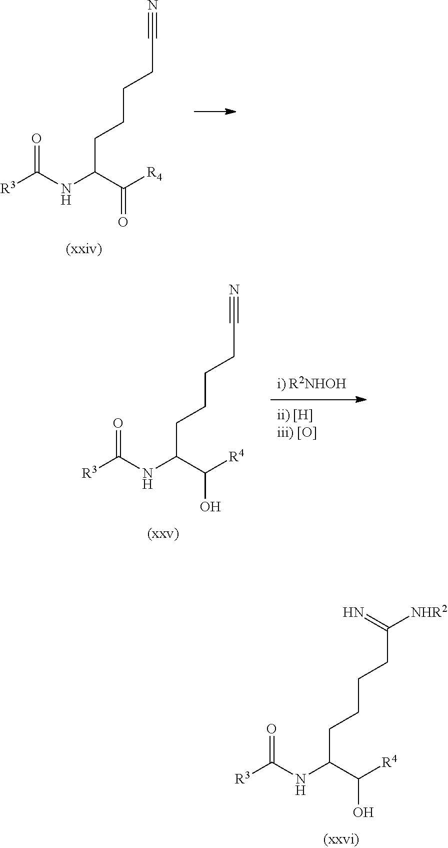

The starting materials and reagents used in preparing the compounds of the present disclosure are either available from commercial suppliers or are prepared by methods known to those skilled in the art following procedures set forth in references such as Fieser and Fieser's Reagents for Organic Synthesis, Vol. 1-28 (Wiley, 2016); March's Advanced Organic Chemistry, 7^{th} Ed. (Wiley, 2013); and Larock's Comprehensive Organic Transformations, 2^{nd} Ed. (Wiley, 1999). The starting materials and the intermediates of the reaction can be isolated and purified if desired using conventional techniques including, but not limited to, filtration, distillation, crystallization, chromatography, and the like. Such materials can be characterized using conventional means, including measuring physical constants and obtaining spectral data.

Unless specified to the contrary, the reactions described herein take place at atmospheric pressure over a temperature range of from about −78° C. to about 250° C. For example, reactions can be conducted at from about 0° C. to about 125° C., or at about room (or ambient) temperature, e.g., about 20° C. In some embodiments, reactions are conducted at about 0° C., 20° C., 25° C., 90° C., 100° C., 110° C., 125° C., 150° C., 175° C., or 200° C. In some embodiments, reactions are conducted starting at a first temperature (e.g., about −78° C. or about 0° C.), and allowed to warm to a higher second temperature (e.g., about 20° C. or about 25° C.). One of skill in the art will appreciate that various modifications to the procedures described herein can be made.

Compounds 1-17, set forth in the following table, can be prepared according to the procedures summarized in Schemes 1-3 and set forth in the Examples below.

| Compound | Compound structure |
|---|---|
| 1 | 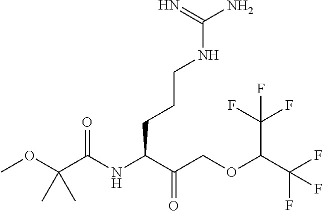 [SMILES: COC(C)(C)C(=O)N[C@@H](CCCNC(=N)N)C(=O)COC(C(F)(F)F)C(F)(F)F] |
| 2 | 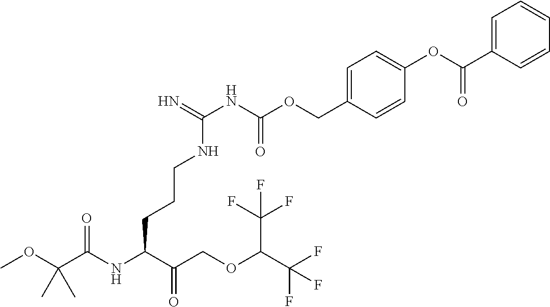 [SMILES: COC(C)(C)C(=O)N[C@@H](CCCNC(=N)NC(=O)OCc1ccc(OC(=O)c2ccccc2)cc1)C(=O)COC(C(F)(F)F)C(F)(F)F] |
| 3 | 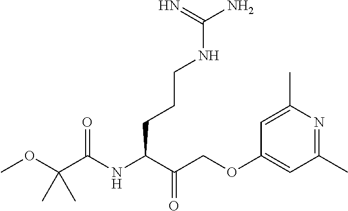 |
| 4 | 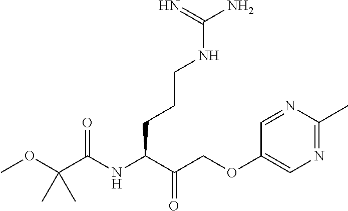 |
| 5 | 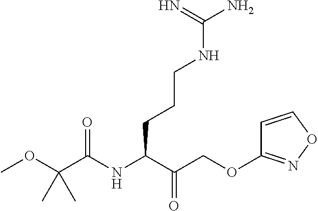 [SMILES: COC(C)(C)C(=O)N[C@@H](CCCNC(=N)N)C(=O)COc1ccon1] |
| 6 | 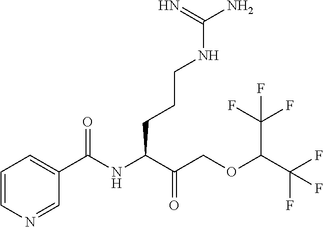 [SMILES: N=C(N)NCCC[C@H](NC(=O)c1cccnc1)C(=O)COC(C(F)(F)F)C(F)(F)F] |
| 7 | 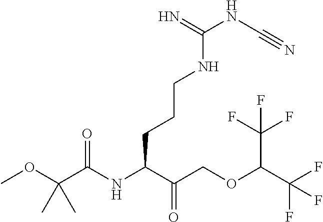 [SMILES: COC(C)(C)C(=O)N[C@@H](CCCNC(=N)NC#N)C(=O)COC(C(F)(F)F)C(F)(F)F] |
| 8 | 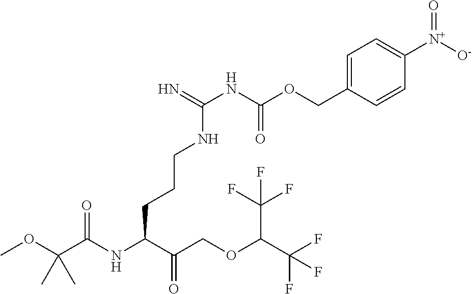 [SMILES: COC(C)(C)C(=O)N[C@@H](CCCNC(=N)NC(=O)OCc1ccc([N+](=O)[O-])cc1)C(=O)COC(C(F)(F)F)C(F)(F)F] |
| 9 | 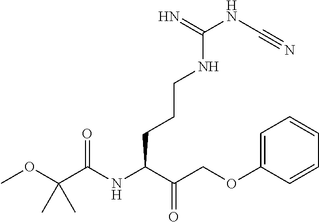 [SMILES: COC(C)(C)C(=O)N[C@@H](CCCNC(=N)NC#N)C(=O)COc1ccccc1] |
| 10 | 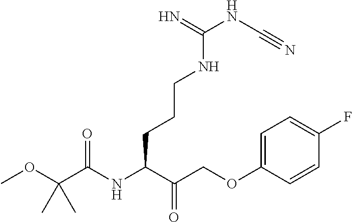 [SMILES: COC(C)(C)C(=O)N[C@@H](CCCNC(=N)NC#N)C(=O)COc1ccc(F)cc1] |
| 11 | 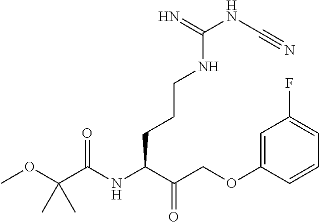 [SMILES: COC(C)(C)C(=O)N[C@@H](CCCNC(=N)NC#N)C(=O)COc1cccc(F)c1] |
| 12 | 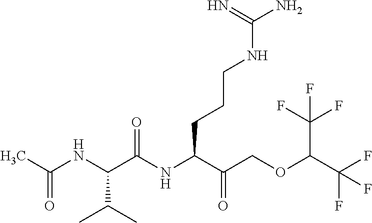 [SMILES: CC(=O)N[C@H](C(=O)N[C@@H](CCCNC(=N)N)C(=O)COC(C(F)(F)F)C(F)(F)F)C(C)C] |
| 13 | 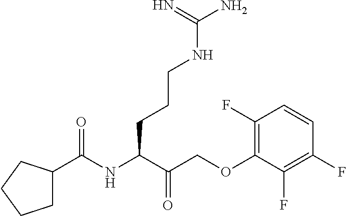 [SMILES: N=C(N)NCCC[C@H](NC(=O)C1CCCC1)C(=O)COc1c(F)ccc(F)c1F] |
| 14 | 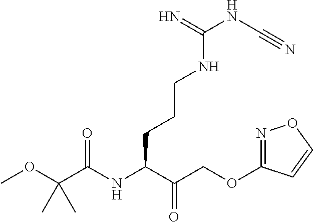 [SMILES: COC(C)(C)C(=O)N[C@@H](CCCNC(=N)NC#N)C(=O)COc1ccon1] |
| 15 | 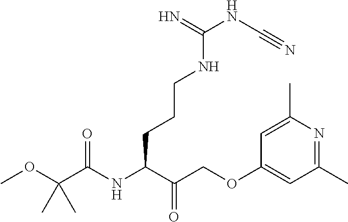 |
| 16 | 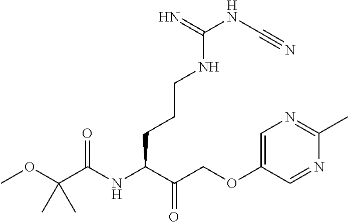 |
| 17 | 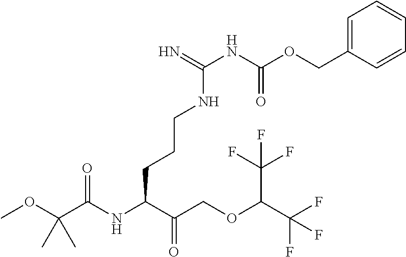 [SMILES: COC(C)(C)C(=O)N[C@@H](CCCNC(=N)NC(=O)OCc1ccccc1)C(=O)COC(C(F)(F)F)C(F)(F)F] |

The compounds described herein are highly active Rgp inhibitors, typically exhibiting Rgp IC₅₀ values in the picomolar, nanomolar, or micromolar range. The term “IC₅₀” indicates how much of a compound is needed to inhibit a given biological process (or component of a process, e.g., an enzyme, cell, cell receptor, or microorganism) by one half (50%). The IC₅₀ value for a particular test compound can be measured as follows. Fifty microliters (μL) of an enzyme such as RgpA or RgpB (1 nM in 50 mM bis-Tris propane [pH 8.0] containing 1% [vol/vol] Triton X-100 and 5 mM 2-mercaptoethanol) is added to columns 1 to 11 of a 96-well plate, and 100 μL is added to column 12. Two μL of the test compound (100 μL in 100% DMSO) is added to column 12, and the sample is mixed three times by pipetting. Then, a doubling dilution is prepared across the plate by serial transfer into adjacent wells. 50 μL of Z-Arg-7-amido-4-methylcoumarin (“Z-Arg-AMC;” 40 μM in buffer) is added to all wells, and the contents are mixed. The reaction is monitored for AMC fluorescence for 15 min at 25° C., and the progress curves are automatically converted to rates by the Fluoroskan Ascent software. The IC₅₀ of a compound can then be determined by constructing a dose-response curve and examining the effect of different concentrations of the compound on reversing the activity of the enzyme. From the dose-response curve, IC₅₀ values can be calculated for a given compound by determining the concentration needed to inhibit half of the maximum biological response of the enzyme.

The method can also be used to assay enzymes including Kgp, trypsin, and cathepsins such as cathepsin B. For Kgp, the substrate can be succinyl-Ala-Phe-Lys-AMC. For trypsin, the buffer can contain 10 mM Tris and 10 mM CaCl₂ (pH 8.0), and the substrate can be Z-Gly-Gly-Arg-AMC. For cathepsin B, the buffer can contain 50 mM sodium phosphate, 1 mM EDTA, and 10 mM 2-mercaptoethanol (pH 6.25), and the substrate can be Z-Arg-Arg-AMC.

In general, the Rgp IC₅₀ values for compounds disclosed herein range from about 0.01 nM to about 100 μM. The Rgp IC₅₀ value for a particular compound can range, for example, from about 0.01 nM to about 0.1 nM, or from about 0.1 nM to about 1 nM, or from about 1 nM to about 100 nM, or from about 100 nM to about 250 nM, or from about 250 nM to about 500 nM, or from about 500 nM to about 750 nM, or from about 750 nM to about 1 μM, or from about 1 μM to about 10 μM, or from about 10 μM to about 25 μM, or from about 25 μM to about 50 μM, or from about 50 μM to about 75 μM, or from about 75 μM to about 100 μM. The Rgp IC₅₀ value for a particular compound can range from about 0.01 nM to about 1 nM, or from about 0.05 nM to about 0.75 nM, or from about 0.1 nM to about 0.5 nM, from about 1 nM to about 100 nM, or from about 20 nM to about 80 nM, or from about 40 nM to about 60 nM, or from about 1 μM to about 100 μM, or from about 20 μM to about 80 μM, or from about 40 μM to about 60 μM.

In some embodiments, an Rgp inhibitor as disclosed herein has an RgpB IC₅₀ of 75 nM or less. In some embodiments, the Rgp inhibitor has an RgpB IC₅₀ of 50 nM or less. In some embodiments, the Rgp inhibitor has an RgpB IC₅₀ of 25 nM or less. In some embodiments, the Rgp inhibitor has an RgpB IC₅₀ of 10 nM or less. In some embodiments, the Rgp inhibitor has an RgpB IC₅₀ of 1 nM or less.

In certain embodiments, Rgp inhibitors according to the present disclosure are selective for Rgp. As used herein, a “selective” Rgp inhibitor is a compound that does not substantially affect the activity of proteases other than RgpA and RgpB when administered at a therapeutically effective dose for treating a disease or condition associated with P. gingivalis infection. Typically, a protease that is not substantially affected by a particular compound exhibits at least 90% of its normal enzymatic activity in the presence of the compound under physiological conditions. Selective Rgp inhibitors include those compounds that do not affect the activity of proteases other than Rgp when administered at a therapeutically effective dose for treating a brain disorder, periodontal disease, diabetes, a cardiovascular disease, arthritis (e.g., rheumatoid arthritis, osteoarthritis, infectious arthritis, or psoriatic arthritis), preterm birth, pneumonia, cancer, a kidney disease, a liver disease, a retinal disorder, or glaucoma associated with P. gingivalis infection. Preferably, selective Rgp inhibitors do not adversely affect the coagulation cascade when administered at therapeutically effective levels.

### III. PHARMACEUTICAL COMPOSITIONS AND ADMINISTRATION OF GINGIPAIN INHIBITORS

In a related embodiments, the pharmaceutical compositions are provided which contain a compound of Formula I, Formula Ia, or Formula Ib or a pharmaceutically acceptable salt thereof and a pharmaceutically acceptable excipient. The pharmaceutical compositions can be prepared by any of the methods well known in the art of pharmacy and drug delivery. In general, methods of preparing the compositions include the step of bringing the active ingredient into association with a carrier containing one or more accessory ingredients. The pharmaceutical compositions are typically prepared by uniformly and intimately bringing the active ingredient into association with a liquid carrier or a finely divided solid carrier or both, and then, if necessary, shaping the product into the desired formulation. The compositions can be conveniently prepared and/or packaged in unit dosage form.

Pharmaceutical compositions containing compounds described herein can be formulated for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is optionally mixed with an inert solid diluent (such as calcium carbonate, calcium phosphate, or kaolin), or as soft gelatin capsules wherein the active ingredient is optionally mixed with water or an oil medium (such as peanut oil, liquid paraffin, or olive oil).

Rgp inhibitors can also be administered topically as a solution, ointment, cream, gel, or suspension, as well as in mouth washes, eye-drops, intranasally-administered formulations, and the like. Still further, transdermal delivery of Rgp inhibitors can be accomplished by means of iontophoretic patches and the like.

Pharmaceutical compositions containing Rgp inhibitors can also be in the form of a sterile injectable aqueous or oleaginous solutions and suspensions. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils can be used as a solvent or suspending medium. For this purpose any bland fixed oil can be employed including synthetic monoglycerides, diglycerides, or triglycerides. In some embodiments, an Rgp inhibitor can be formulated with a polymer such as Pluronic F127 and delivered subcutaneously. Pluronic is a hydrogel that solidifies at body temperature and can provide extended drug delivery over periods of time lasting from days to weeks.

Aqueous suspensions can contain one or more Rgp inhibitors in admixture with excipients including, but not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Dispersible powders and granules (suitable for preparation of an aqueous suspension by the addition of water) can contain one or more Rgp inhibitors in admixture with a dispersing agent, wetting agent, suspending agent, or combinations thereof. Oily suspensions can be formulated by suspending an Rgp inhibitor in a vegetable oil (e.g., arachis oil, olive oil, sesame oil or coconut oil), or in a mineral oil (e.g., liquid paraffin). Oily suspensions can contain one or more thickening agents, for example beeswax, hard paraffin, or cetyl alcohol. These compositions can be preserved by the addition of an anti-oxidant such as ascorbic acid.

The pharmaceutical compositions can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

The use of hybrid molecules to promote active transport or nanoparticles can be used in certain embodiments to increase blood brain barrier transport. For example liposomes, proteins, engineered peptide compounds or antibodies that bind to the receptors that transport proteins across the blood brain barrier including LPR-1 receptor, transferrin receptor, EGF-like growth factor or glutathione transporter can be used to increase penetration into the brain. Physical techniques including osmotic opening, ultrasound, lasers, sphenopalantine ganglion stimulation, direct intracranial, intrathecal, or intraventricular delivery via a pump can be used.

Pharmaceutical compositions can also include one or more additional active agents useful in the treatment of conditions associated with P. gingivalis infection. In certain embodiments, the pharmaceutical compositions contain one or more Rgp inhibitors as described herein in combination with one or more additional active agents for treatment of Alzheimer's disease. Several therapeutics are in development and in clinical use for treatment of Alzheimer's disease. Therapeutic strategies include lowering circulating levels of β-amyloid and tau (as described in more detail below), stabilizing microtubules, removing atherosclerotic plaques, modulating autophagy, modulating neurotransmitter levels, and inhibiting GABA(A) α5 receptors. Such therapeutics can maintain and/or restore cognitive function in subjects with Alzheimer's disease; slow the decline of cognitive function; and promote neuroplasticity and recovery of the brain.

Active agents that can be combined with Rgp inhibitors in pharmaceutical compositions include, but are not limited to, antibiotics (i.e., bacteriocidal compounds and bacteriostatic compounds), cholinesterase inhibitors, alpha-7 nicotinic receptor modulators, serotonin modulators, NMDA modulators, Aβ-targeted therapies, ApoE-targeted therapies, microglia-targeted therapies, blood/brain barrier-targeted therapies, tau-targeted therapies, complement-targeted therapies, and anti-inflammatories.

Any suitable antibiotic can be combined with one or more Rgp inhibitors in the pharmaceutical compositions. In certain embodiments, the pharmaceutical composition contains one more Rgp inhibitors and an antibiotic having a P. gingivalis MIC₅₀ of less than 25 μg/ml. For example, the P. gingivalis MIC₅₀ of the antibiotic can be less than 20 μg/ml, less than 15 μg/ml, less than 10 μg/ml, less than 8 μg/ml, less than 6 μg/ml, or less than 5 μg/ml. In some embodiments, the P. gingivalis MIC₅₀ of the antibiotic is less than 1 μg/ml. In some embodiments, the P. gingivalis MIC₅₀ of the antibiotic is less than 0.2 μg/ml.

Examples of bacteriocidal and bacteriostatic compounds include, but are not limited to: quinolones (e.g., moxifloxacin, gemifloxacin, ciprofloxacin, oflaxacin, trovafloxacin, sitafloxacin, and the like), β-lactams (e.g., penicillins such as amoxicillin, amoxacilin-clavulanate, piperacillin-tazobactam, penicillin G, and the like; and cephalosporins such as ceftriaxone and the like), macrolides (e.g., erythromycin, azithromycin, clarithromycin, and the like), carbapenems (e.g., doripenem, imipenem, meropinem, ertapenem, and the like), thiazolides (e.g., tizoxanidine, nitazoxanidine, RM 4807, RM 4809, and the like), tetracyclines (e.g., tetracycline, minocycline, doxycycline, eravacycline, and the like), clindamycin, metronidazole, and satranidazole. Bacteriocidal and bacteriostatic compounds also include agents that inhibit or otherwise interfere with formation of biofilms by anaerobic, gram-negative bacteria; such agents include oxantel, morantel, thiabendazole, and the like. Compositions can contain one or more Rgp inhibitors as described herein with one or more (e.g., two, three, four, five, six, or more) bacteriocidal/bacteriostatic compounds. Compositions containing bacteriocidal/bacteriostatic compounds can further contain a chlorhexidine (e.g., chlorhexidine digluconate) alone or in combination with a zinc compound (e.g., zinc acetate), can also be used in combination with the administered antibiotics.

In some embodiments, a combination of a penicillin (e.g., amoxicillin) and metronidazole or a combination of penicillin (e.g., amoxicillin), metronidazole and a tetracycline is used. In some embodiments, the antibiotic is selected from minocycline, doxycycline, metronidazole, amoxicillin, clindamycin, augmentin, satranidazole, and combinations thereof.

Examples of suitable cholinesterase inhibitors include, but are not limited to, donepezil, donepezil/memantine, galantamine, rivastigmine, and tacrine, as well as pharmaceutically acceptable salts thereof. Examples of suitable serotonin modulators include, but are not limited to, idalopirdine, RVT-101, citalopram, escitalopram, fluoxetine, fluvoxamine, paroxetine, and sertraline, as well as pharmaceutically acceptable salts thereof. Examples of suitable alpha-7 nicotinic receptor modulators include, but are not limited to, alpha-7 agonists such as encenicline and APN1125. Suitable NMDA modulators include, but are not limited to, NMDA receptor antagonists such as memantine and derivatives thereof

Pharmaceutical compositions can also contain active agents that are directed to biomolecular targets associated with neurological diseases. Such targets include beta amyloid peptides (also referred to as beta amyloid, abeta, or Aβ), apolipoprotein E (also referred to as ApoE), and microtubule-associated tau (also referred to as tau proteins, or simply as tau).

Aβ-targeted therapies include inhibitors of Aβ production (such as beta-secretase inhibitors, gamma-secretase inhibitors, alpha-secretase activators), inhibitors of Aβ aggregation, inhibitors of Aβ oligomerization, and up-regulators of Aβ clearance, among others (see, e.g., Jia, et al. BioMed Research International, 2014. Article ID 837157, doi:10.1155/2014/837157). Examples of Aβ-targeted therapies include but are not limited to, antibodies, pioglitazone, begacestat, atorvastatin, simvastatin, etazolate, and tramiprosate, as well as pharmaceutically acceptable salts thereof.

Examples of ApoE-targeted therapies include, but are not limited to retinoid X receptor agonists (see, Cramer, et al., Science 2012. 335(6075): 1503-1506) and others described by Liu et al. (Nat Rev Neurol. 2013. 9(2): 106-118). Tau-targeted therapies include, but are not limited to, methylthioninium, leuco-methylthioninium, antibodies and those described by Lee, et al. (Cold Spring Harb Perspect Med 2011; 1:a006437).

Pharmaceutical compositions can also contain complement-targeted therapies. Such therapies target components of the complement system involved in the innate immune response. Complement targeted therapies include, but are not limited to, those described by Ricklin and Lambris (Nat. Biotechnology 2007. 25(11): 1265-1275).

Examples of suitable anti-inflammatories include, but are not limited to, NSAIDs such as apazone, diclofenac, ibuprofen, indomethacin, ketoprofen, nabumetone, naproxen, piroxicam, and sulindac, as well as pharmaceutically acceptable salts thereof.

### IV. METHODS FOR INHIBITING GINGIPAINS AND TREATING CONDITIONS ASSOCIATED WITH P. GINGIVALIS INFECTION

In another embodiment, methods of inhibiting a gingipain are provided. The methods include contacting the gingipain with an effective amount of a compound as described herein. In certain embodiments, the gingipain is an arginine gingipain (e.g., RgpA, RgpB, or a variant containing one or more amino acid substitutions, deletions, and/or other peptide sequence variations). Inhibiting the gingipain generally includes contacting the gingipain with an amount of the compound sufficient to reduce the activity of the gingipain as compared to the gingipain activity in the absence of the compound. For example, contacting the gingipain with the gingipain inhibitor can result in from about 1% to about 99% gingipain inhibition (i.e., the activity of the inhibited gingipain ranges from 99% to 1% of the gingipain activity in the absence of the compound). The level of gingipain inhibition can range from about 1% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%. The level of gingipain inhibition can range from about 5% to about 95%, or from about 10% to about 90%, or from about 20% to about 80%, or from about 30% to about 70%, or from about 40% to about 60%. In some embodiments, contacting the gingipain with a compound as described herein will result in complete (i.e., 100%) gingipain inhibition.

As described above, infection with P. gingivalis and gingipain activity have been linked to the development of periodontal disease, Alzheimer's disease and other brain disorders, cardiovascular disease, diabetes, cancer, liver disease, kidney disease, preterm birth, arthritis, pneumonia and other disorders. See: Bostanci, et al. FEMS Microbiol Lett, 2012. 333(1): 1-9; Ghizoni, et al. J Appl Oral Sci, 2012. 20(1): 104-12; Gatz, et al. Alzheimers Dement, 2006. 2(2): 110-7; Stein, et al. J Am Dent Assoc, 2007. 138(10): 1314-22; quiz 1381-2; Noble, et al. J Neurol Neurosurg Psychiatry, 2009. 80(11): 1206-11; Sparks Stein, et al. Alzheimers Dement, 2012. 8(3): 196-203; Velsko, et al. PLoS ONE, 2014. 9(5): e97811; Demmer, et al. J Dent Res, 2015. 94(9S): 201-S-11S; Atanasova and Yilmaz. Molecular Oral Microbiology, 2014. 29(2): 55-66; Yoneda, et al. BMC Gastroenterol, 2012. 12: 16.

Extracellular proteases produced by P. gingivalis, including Arginine Gingipain A (RgpA), Arginine Gingipain B (RgpB), and Lysine Gingipain (Kgp), can also degrade a broad range of proteins in connective tissue and plasma (e.g., collagen, immunoglobulins, and proteinase inhibitors, etc.). Gingipains can enter systemic circulation and/or synoviocytes and chondrocytes, and they can also cause disruption to the kallikrein-kinin cascade, blood coagulation, and host defense systems. Patients with gingipains in their joints and circulatory system may be subject to gingipain-induced death of synovial cells and/or chondrocytes, contributing to osteoarthritis. As used herein, the term “osteoarthritis” refers to a chronic degenerative joint disease that results from breakdown of joint cartilage, synovial tissue, and underlying bone. It has recently been discovered that RgpB and Kgp can infiltrate human and dog joints, contributing to the development of osteoarthritis. It is believed that P. gingivalis and gingipains can infiltrate joint tissues via a number of routes. Gingipains can be secreted, transported to outer membrane surfaces of P. gingivalis, or released in outer membrane vesicles by the bacterium. P. gingivalis has previously been identified in periodontal tissues, coronary arteries, aorta, and recently, the liver—release of P. gingivalis and/or gingipains from any of these niches into the systemic circulation could result in translocation of P. gingivalis and/or gingipains to the joints. See: Travis, et al. Adv Exp Med Biol, 2000. 477: 455-65; Byrne, et al. Oral Microbiol Immunol, 2009. 24(6): 469-77; Mahendra, et al. J Maxillofac Oral Surg, 2009. 8(2): 108-13; Stelzel. Periodontol, 2002. 73(8): 868-70; Ishikawa, et al. Biochim Biophys Acta, 2013. 1832(12): 2035-2043.

Rgp inhibitors can be used to treat diseases and conditions, such as brain disorders, caused by or otherwise affected by P. gingivalis. Accordingly, another aspect of the invention provides a method of treating a disease or condition associated with P. gingivalis infection. The method includes administering an effective amount of a compound or a composition, as described above, to a subject in need thereof.

In certain embodiments, compounds according to the present disclosure inhibit active Rgp in the brain of a mammal, e.g., a human or an animal (e.g., a dog), and are cytoprotective or neuroprotective. By “neuroprotective,” it is meant that the compounds prevent aberrant changes to neurons or death of neurons. Compounds of the invention are therefore useful, e.g., in treatment of a brain disorder (e.g., a neurodegenerative disease (e.g., Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, cerebrovascular disease, Lewy Body disease, multiple system atrophy, schizophrenia and depression, etc.), diabetes, cardiovascular disease, arthritis (e.g., rheumatoid arthritis, osteoarthritis, infectious arthritis, or psoriatic arthritis), retinal disorders (e.g., age related macular degeneration) and glaucoma.

In some embodiments, the disease or condition is selected from a brain disorder, periodontal disease, diabetes, a cardiovascular disease, rheumatoid arthritis, osteoarthritis, preterm birth, pneumonia, cancer, a kidney disease, a liver disease, a retinal disorder, and glaucoma.

In some embodiments, the disease or condition is a brain disorder.

In some embodiments, the brain disorder is selected from Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, cerebrovascular disease, Lewy Body disease, multiple system atrophy, schizophrenia, and depression.

In some embodiments, the brain disorder is Alzheimer's disease.

In some embodiments, the method further includes administering to the subject one or more active agents selected from a cholinesterase inhibitor, a serotonin modulator, an NMDA modulator, an Aβ targeted therapy, an ApoE targeted therapy, a microglia targeted therapy, a blood brain barrier targeted therapy, a tau targeted therapy, a complement targeted therapy, and an anti-inflammatory.

In some embodiments, the disease or condition is periodontal disease. In some embodiments, the disease or condition is a liver disease. In some embodiments, the liver disease is non-alcoholic steatohepatitis. In some embodiments, the disease or condition is a retinal disorder. In some embodiments, the retinal disorder is age-related macular degeneration.

In some embodiments, the disease or condition is cancer. In some embodiments, the cancer is breast cancer, oral cancer, pancreatic cancer, or glioblastoma multiforme.

Rgp inhibitors can be administered at any suitable dose in the methods provided herein. In general, an Rgp inhibitor is administered at a dose ranging from about 0.1 milligrams to about 1000 milligrams per kilogram of a subject's body weight (i.e., about 0.1-1000 mg/kg). The dose of Rgp inhibitor can be, for example, about 0.1-1000 mg/kg, or about 1-500 mg/kg, or about 25-250 mg/kg, or about 50-100 mg/kg. The dose of Rgp inhibitor can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. The dosages can be varied depending upon the requirements of the patient, the severity of the disorder being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the disease or condition.

Rgp inhibitors can be administered for periods of time which will vary depending upon the nature of the particular disorder, its severity, and the overall condition of the subject to whom the Rgp inhibitor is administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a subject can be monitored for changes in his or her condition and for alleviation of the symptoms of the disorder. The dosage of the Rgp inhibitor can either be increased in the event the subject does not respond significantly to a particular dosage level, or the dose can be decreased if an alleviation of the symptoms of the disorder is observed, or if the disorder has been remedied, or if unacceptable side effects are seen with a particular dosage.

A therapeutically effective amount of an Rgp inhibitor can be administered to the subject in a treatment regimen comprising intervals of at least 1 hour, or 6 hours, or 12 hours, or 24 hours, or 36 hours, or 48 hours between dosages. Administration can be conducted at intervals of at least 72, 96, 120, 144, 168, 192, 216, or 240 hours (i.e., 3, 4, 5, 6, 7, 8, 9, or 10 days). In certain embodiments, administration of one or more Rgp inhibitors is conducted in a chronic fashion over periods ranging from several months to several years. Accordingly, some embodiments provide a method of treating a disease or condition associated with P. gingivalis infection as described above, wherein the compound is administered to the subject for at least one year. In some embodiments, the compound is administered to the subject for at least 10 years. In some embodiments, the compound is administered to the subject for at least 60 years.

Administration of Rgp inhibitors according to the methods provided herein typically results in the reduction of circulating levels of active Rgp in a subject and/or the reduction of active Rgp in the brain. In certain embodiments, administration of an Rgp inhibitor results in at least a 20% reduction of circulating levels of active Rgp and/or at least a 20% reduction of active Rgp in the brain. For example, the circulating levels of Rgp and/or the levels of Rgp in the brain are preferably reduced by from about 25% to about 95%, or from about 35% to about 95%, or from about 40% to about 85%, or from about 40% to about 80% as compared to the corresponding levels of Rgp 24 hours prior to the first administration of the Rgp inhibitor.

Rgp inhibitors can be administered alone or in combination with one or more additional therapeutically active agents, as described above. The one or more additional therapeutically effective agents include, e.g., (i) a pharmaceutically acceptable agent which inhibits RgpA, RgpB, and/or Kgp production, translocation of RgpA, RgpB, and/or Kgp into systemic circulation or brain, and/or pathological (e.g., neurotoxic effects) of RgpA, RgpB, and/or Kgp in a mammal; (ii) an antibacterial agent which is bacteriostatic or bacteriocidal with respect to P. gingivalis; (iii) one or more antibodies which bind to RgpA, RgpB and/or Kgp (e.g., 18E6, which binds to the first half of the immunoglobulin domain of RgpB; Kgp-specific monoclonal antibody, 7B9, which recognizes an epitope within the Kgp catalytic domain; the RgpA antibody 61Bg 1.3, humanized versions of any of the foregoing, etc.); (iv) epitopes of antibodies which bind to RgpA, RgpB and/or Kgp or other proteins expressed by P. gingivalis; and (v) combinations of any of the foregoing.

The additional therapeutically active agents also include Aβ peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA(A) α5 receptors inhibitors, and additional agents that help maintain and/or restore cognitive function and functional deficits of Alzheimer's disease, and/or slow down decline in cognitive functions and functional deficits in Alzheimer's disease.

Pharmaceutical compositions can contain one or more Rgp inhibitors as described herein in combination with ritonavir (RTV), which can increase bioavailability and increase blood brain barrier penetration. For example, ritonavir is commonly combined with oral peptidic HIV protease inhibitors to increase plasma levels by inhibiting the P450 3A4 enzyme and thus decreasing first-pass metabolism (see, Walmsley, et al., N Engl J Med, 2002. 346(26): 2039-46). In addition, RTV binds to P-glycoprotein, a transmembrane efflux pump that is found in many tissues, including the blood brain barrier, allowing co-administered compounds better access to the brain (see, Marzolini, et al., Mol Pharm, 2013. 10(6): 2340-9). Therefore, a combination of RTV and Rgp inhibitors can be used to increase plasma concentrations and brain levels of the gingipain inhibitors. As described in U.S. Pat. No. 9,758,473, for example, oral administration of RTV 15 minutes prior to the Kgp inhibitor Kyt-36 increases the half-life therefore it is expected that RTV will also increase the half-life of other gingipain inhibitors.

In some embodiments, compounds described herein can be administered with natural gingipain inhibitors including melabaricone C, isolated from nutmeg or polyphenolic compounds derived from plants, such as cranberry, green tea, apple, and hops can be administered in conjunction for treatment or prevention of brain disorders. Naturally and unnaturally occurring antimicrobial peptides including: η-casein peptide (109-137) 34, histatin 5, and CL(14-25), CL(K25A) and CL(R24A, K25A), can also be administered in conjunction with the Rgp inhibitors. (see, e.g., Taniguchi et al., Biopolymers, 2014. 102(5): 379-89).

Rgp inhibitors as described herein can be administered with antibodies targeting gingipains or other P. gingivalis proteins. Antibodies may rely on damage to the blood brain barrier for access to the brain or peripheral interference with gingipains and P. gingivalis propagation. Antibodies can also help to stimulate the efficacy of the immune system in clearing the bacteria. New or existing antibodies to RgpA, RgpB, or Kgp can be utilized including 18E6 and 7B9. An RgpA antibody 61BG 1.3 has previously demonstrated efficacy topically in prevention of recolonization by P. gingivalis after periodontal treatment. See, Booth et al., Infect Immun, 1996. 64(2): 422-7. Antibodies would preferably be humanized for use in humans. Methods known to those in the field for delivery of biologics to improve half- life and brain penetration can be used including, but not limited to, intravenous delivery, subcutaneous delivery, intranasal delivery, intrathecal delivery, intra-articular delivery, vector transport, and direct brain delivery.

The methods provided herein also encompass administration of Rgp inhibitors as described herein with one or more of the following additional therapeutically active agents or pharmaceutically acceptable salts thereof: an arginine derivative; histatin 5; baculovirus p35; a single point mutant of cowpox viral cytokine-response modifier (CrmA (Asp>Lys)); phenylalanyl-ureido-citrullinyl-valyl-cycloarginal (FA-70C1); (acycloxy)methyl ketone (Cbz-Phe-Lys-CH₂OCO-2,4,6-Me₃Ph); peptidyl chloro-methyl ketones (e.g., chloromethyl ketone derivatives of arginine, chloromethyl ketone derivatives of lysine, and the like); fluoro-methyl ketones; bromo-methyl ketones; ketopeptides; 1-(3-phenylpropionyl)piperidine-3(R,S)-carboxylic acid [4-amino-1(S)-(benzothiazole-2-carbonyl)butyl]amide (A71561); azapeptide fumaramide; aza-peptide Michael acceptors; benzamidine compounds; acyclomethylketone; activated factor X inhibitors (e.g., DX-9065a); cranberry nondialyzable fraction; cranberry polyphenol fraction; pancreatic trypsin inhibitor; Cbz-Phe-Lys-CH₂O—CO-2,4,6-Me₃—Ph; E-64; chlorhexidine; zinc (e.g., zinc acetate); or a combination of two, three or more of any of foregoing. In some of these embodiments, Zn can enhance potency and selectivity of the compounds (e.g., chlorhexidine, benzamidine, etc.) used in the methods.

An Rgp inhibitor as described herein can be administered in the same composition as an additional therapeutically active agent. Alternatively, the additional therapeutically active agent can be administered separately before, concurrently with, or after administration of the Rgp inhibitor.

### V. EXAMPLES

### Example 1. Preparation of (S)-N-(6-guanidino-1-((1,1,1,3,3,3-hexafluoropropan-2-yl)oxy)-2-oxohexan-3-yl)-2-methoxy-2-methylpropanamide (1)

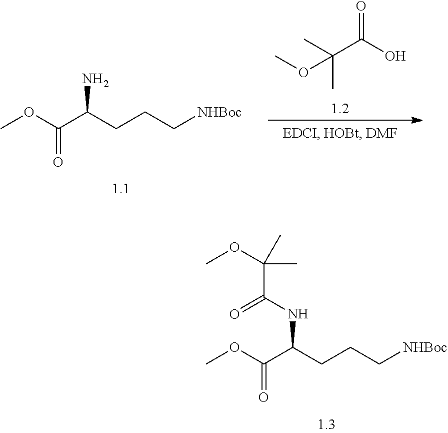

Compound 1.3: To a mixture of compound 1.2 (12.53 g, 106.1 mmol, 1 eq) and HOBt (15.77 g, 116.71 mmol, 1.1 eq) in DMF (300 mL) was added EDCI (22.37 g, 116.71 mmol, 1.1 eq) in one portion at 0° C. under N₂. The mixture was stirred at 0° C. for 60 min, then the mixture was added compound 1.1 (30 g, 106.1 mmol, leg, HCl) and DIPEA (41.14 g, 318.29 mmol, 55.44 mL, 3 eq), then the mixture was stirred at 0° C. for 6 hours. The reaction mixture was diluted with H₂O 100 mL and extracted with EtOAc (100 mL×3).

The combined organic layers were washed with H₂O (200 mL×3) and brine 300 mL, dried over Na₂SO₄, filtered and concentrated under reduced pressure. The resulting residue was purified by column chromatography (SiO₂, petroleum ether/ethyl acetate=10:1 to 1:1) to provide compound 1.3 (43 g, crude) as colorless oil. LCMS (ESI): m/z: [M+H] calcd for C₁₆H₃₁O₆N₂: 347; found: 347, RT=1.061 min.

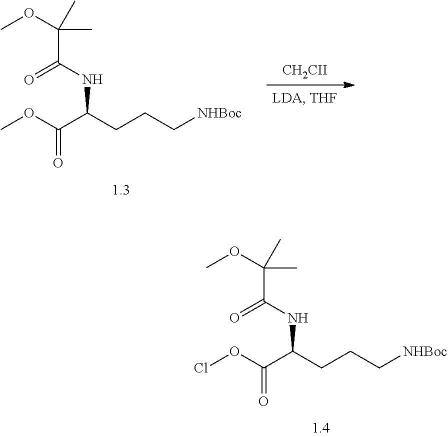 [SMILES: COC(=O)[C@H](CCCNC(=O)OC(C)(C)C)NC(=O)C(C)(C)OC ; "CH2CII" ; "LDA, THF" ; COC(C)(C)C(=O)N[C@@H](CCCNC(=O)OC(C)(C)C)C(=O)OCl]

Compound 1.4: To a solution of DIPA (20.89 g, 206.4 mmol, 29.17 mL, 5.5 eq) in THF (100 mL) was added n-BuLi (2.5 M, 82.56 mL, 5.5 eq) at 0° C., the mixture was stirred at 0° C. for 30 min. The mixture was added a solution of chloro (iodo) methane (36.41 g, 206.40 mmol, 14.98 mL, 5.5 eq) and compound 1.3 (13 g, 37.53 mmol, 1 eq) in THF (100 mL) at −78° C. The mixture was stirred at −78° C. for 3 h. The residue was diluted with H₂O 100 mL and extracted with EtOAc (100 mL×3). The combined organic layers were washed with H₂O (100 mL×3). The combined organic layers were washed with brine (200 mL), dried over Na₂SO₄, filtered and concentrated under reduced pressure. The resulting residue was purified by column chromatography (SiO₂, petroleum ether/ethyl acetate=10:1 to 1:1) to afford compound 1.4 (13.71 g, crude) as yellow oil.

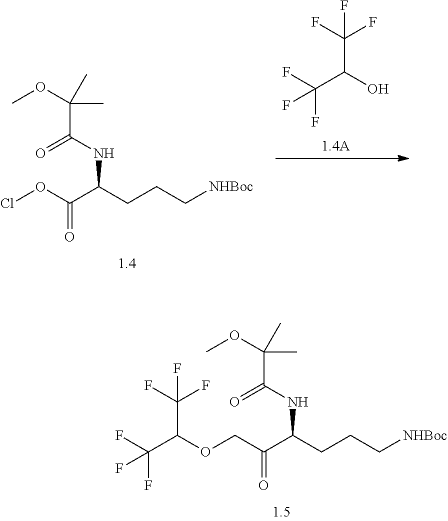

Compound 1.5: To a solution of compound 1.4 (13.71 g, 37.57 mmol, 1 eq) and compound 1.4A (6.31 g, 37.57 mmol, 1 eq) in DMF (100 mL) was added K₂CO₃ (15.58 g, 112.71 mmol, 3 eq) and KI (6.24 g, 37.57 mmol, 1 eq). The mixture was stirred at 25° C. for 1 hr. The residue was diluted with H₂O 100 mL and extracted with EtOAc (100 mL×3). The combined organic layers were washed with H₂O (100 mL×3). The combined organic layers were washed with brine (200 mL), dried over Na₂SO₄, filtered and concentrated under reduced pressure. The resulting residue was purified by column chromatography (SiO₂, petroleum ether/ethyl acetate=10:1 to 1:1) to provide compound 1.5 (5.5 g, 11.08 mmol, 29.49% yield) as yellow oil. ¹H NMR (400 MHz, CHLOROFORM-d), δ ppm 1.36-1.42 (m, 6 H), 1.43-1.51 (m, 8 H), 1.52-1.59 (m, 2 H), 1.89-1.99 (m, 1 H), 3.16 (br d, J=5.99 Hz, 2 H), 3.32 (s, 2 H), 4.35 (dt, J=11.31, 5.59 Hz, 1 H), 4.62 (br d, J=3.06 Hz, 2 H), 4.67-4.75 (m, 1 H), 7.20 (br d, J=7.34 Hz, 1 H).

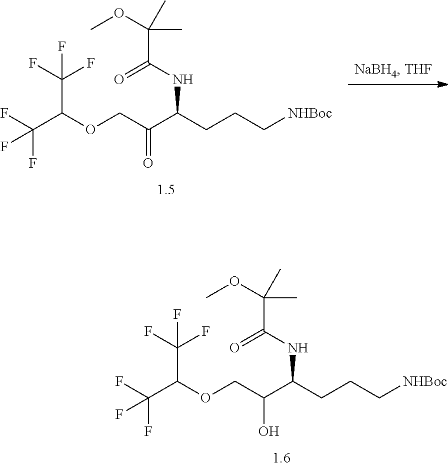

Compound 1.6: To a solution of compound 1.5 (5.5 g, 11.08 mmol, 1 eq) in THF (60 mL) was added NaBH₄ (838.28 mg, 22.16 mmol, 2 eq). The mixture was stirred at 25° C. for 1 hr. The residue was diluted with H₂O (50 mL) and extracted with EtOAc (50 mL×3). The combined organic layers were washed with brine (200 mL), dried over Na₂SO₄, filtered and concentrated under reduced pressure. Compound 1.6 (6 g, crude) was obtained as colorless oil and used without further purification.

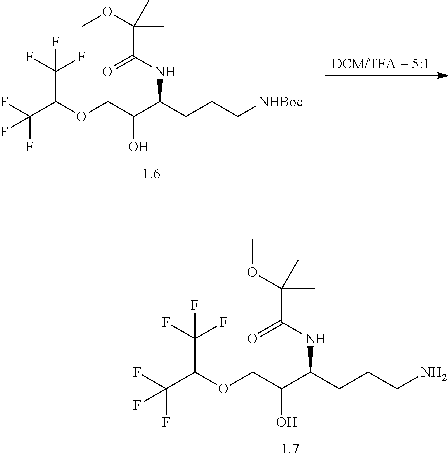

Compound 1.7: To a solution of compound 1.6 (6 g, 12.04 mmol, 1 eq) in DCM (50 mL) was added TFA (15.40 g, 135.06 mmol, 10 mL, 11.22 eq). The mixture was stirred at 25° C. for 1 hr, filtered, and concentrated under reduced pressure. Compound 1.7 (9.7 g, crude) was obtained as colorless oil and used without further purification.

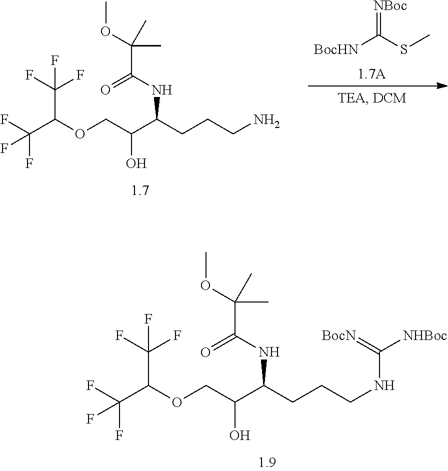

Compound 1.9: To a solution of compound 1.7 (360 mg, 722.23 umol, 1 eq) and compound 1.7A (419.44 mg, 1.44 mmol, 2 eq) in DCM (4 mL) was added TEA (219.25 mg, 2.17 mmol, 301.58 μL, 3 eq). The mixture was stirred at 25° C. for 10 hr. The residue was diluted with H₂O (5 mL) and extracted with DCM (5 mL×3). The combined organic layers were washed with H₂O (5 mL×3). The combined organic layers were washed with brine (10 mL), dried over Na₂SO₄, filtered, and concentrated under reduced pressure. The resulting residue was purified by column chromatography (SiO₂, petroleum ether/ethyl acetate=10:1 to 1:1) to provide compound 1.9 (110 mg, 171.71 μmol, 23.78% yield) as a colorless oil.

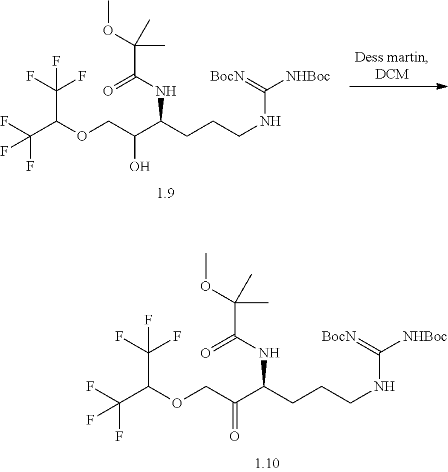

Compound 1.10: To a solution of compound 1.9 (109.65 mg, 171.17 μmol, 1 eq) in DCM (1 mL) was added Dess-Martin periodinane (145.20 mg, 342.34 umol, 105.99 μL, 2eq). The mixture was stirred at 25° C. for 10 hr, filtered, and concentrated under reduced pressure. The resulting residue was purified by prep-HPLC (column: Waters Xbridge 150×25 5 μm; mobile phase: [water (0.04%NH₃/H₂O, 10 mM NH₄HCO₃)-ACN]; B %: 50%-85%, 10 min) to afford compound 1.10 (30 mg, 46.98 μmol, 27.44% yield) as avcolorless oil.

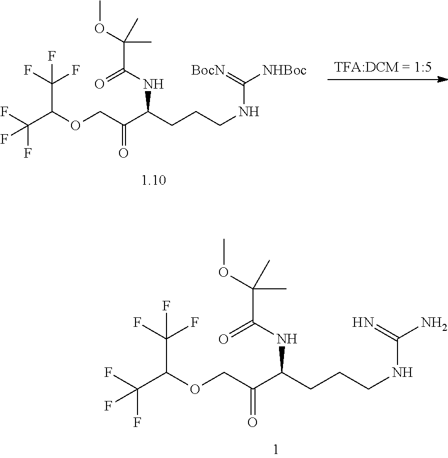

Compound 1: To a solution of compound 1.10 (30 mg, 46.98 μmol, 1 eq) in DCM (5 mL) was added TFA (1.54 g, 13.51 mmol, 1mL, 287.5 eq). The mixture was stirred at 25° C. for 2 hr, filtered and concentrated under reduced pressure to obtain the desired product as a colorless oil. LCMS (ESI): m/z: [M+H]⁺ calcd for C₁₅H₂₅O₄F₆N₄: 439; found 439, RT=2.537 min. ¹H NMR (400 MHz, METHANOL-d4), δ ppm 1.23-1.34 (m, 6 H), 1.42-1.63 (m, 3 H), 1.84-1.97 (m, 1 H), 3.20 (br d, J=4.77 Hz, 2 H), 3.23 (br s, 3 H), 4.48 (dd, J=8.93, 4.03 Hz, 1 H), 4.52-4.69 (m, 2 H), 4.86-4.99 (m, 1 H).

### Example 2. Preparation of (S)-4-(10-(2-((1,1,1,3,3,3-hexafluoropropan-2-yl)oxy)acetyl)-5-imino-13,13-dimethyl-3,12-dioxo-2,14-dioxa-4,6,11-triazapentadecyl)phenyl benzoate (2)

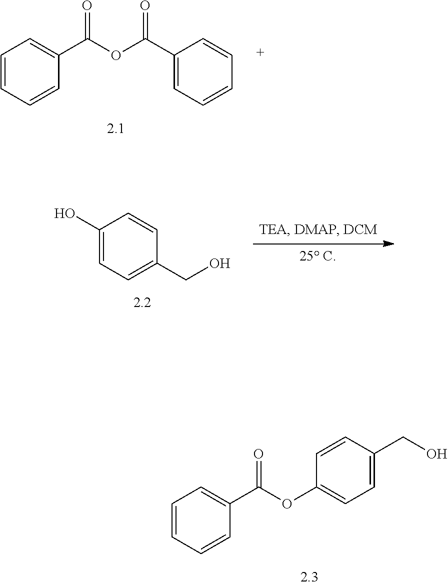 [SMILES: O=C(OC(=O)c1ccccc1)c1ccccc1 ; OCc1ccc(O)cc1 ; "25° C." ; "TEA, DMAP, DCM" ; O=C(Oc1ccc(CO)cc1)c1ccccc1]

Compound 2.3: To a solution of compound 2.2 (500 mg, 4.03 mmol, 1 eq) in DCM (7 mL) was added TEA (815.15 mg, 8.06 mmol, 1.12 mL, 2 eq), DMAP (984.16 mg, 8.06 mmol, 2 eq), and compound 2.1 (911.20 mg, 4.03 mmol, 759.33 μL, 1 eq). The mixture was stirred at 25° C. for 1 hr. The reaction mixture was quenched by addition of H₂O (10 mL), and then extracted with DCM (10 mL×3). The combined organic layers were washed with 1N HCl (10 mL) and saturated brine (10 mL), dried over Na₂SO₄, filtered, and concentrated under reduced pressure to provide a residue which was purified by MPLC (SiO₂, petroleum ether/ethyl acetate=10/1 to 1:1) to afford compound 2.3 (470 mg, 2.06 mmol, 51.12% yield) as a white solid.

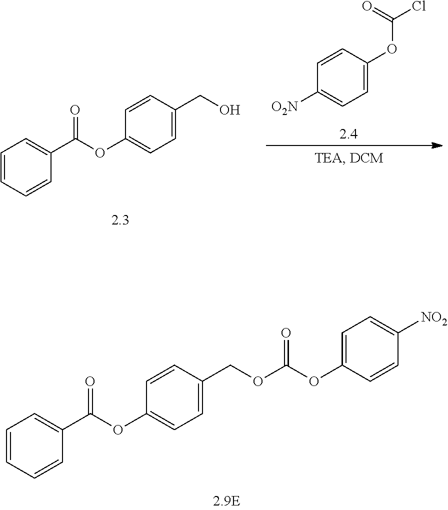

Compound 2.9E: To a solution of compound 2.3 (100 mg, 438.13 μmol, 1 eq) in DCM (3 mL) was added compound 2.4 (88.31 mg, 438.13 μmol, 1 eq) and TEA (88.67 mg, 876.26 μmol, 121.97 μL, 2 eq). The mixture was stirred at 0° C. for 1 hr. The reaction mixture was quenched by addition of H₂O (5 mL) and then extracted with DCM (5 mL×3).

The combined organic layers were washed with saturated brine (5 mL), dried over Na₂SO₄, filtered and concentrated under reduced pressure to afford compound 2.9E (160 mg, crude) as a white solid.

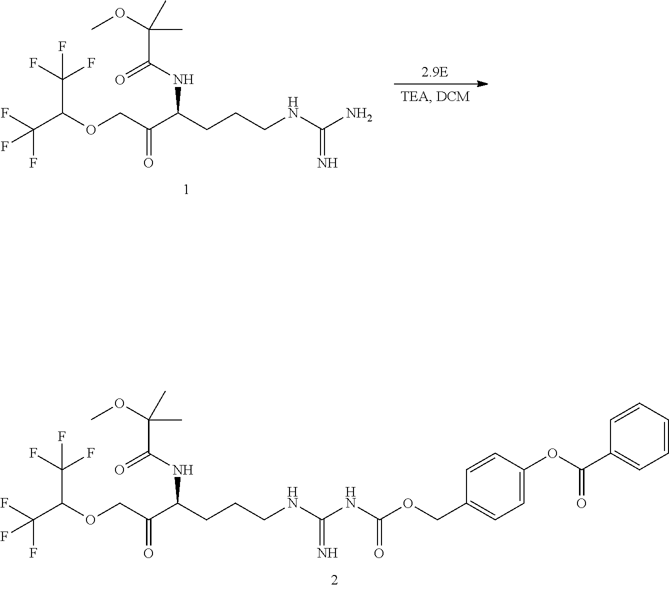 [SMILES: COC(C)(C)C(=O)N[C@@H](CCCNC(=N)N)C(=O)COC(C(F)(F)F)C(F)(F)F ; "2.9E" ; "TEA, DCM" ; COC(C)(C)C(=O)N[C@@H](CCCNC(=N)NC(=O)OCc1ccc(OC(=O)c2ccccc2)cc1)C(=O)COC(C(F)(F)F)C(F)(F)F]

Compound 2: To a solution of compound 1 (113 mg, 257.78 μmol, 1 eq) and compound 2.9E (101.40 mg, 257.78 μmol, 1 eq) in DCM (3 mL) was added TEA (78.25 mg, 773.33 μmol, 107.64 μL, 3 eq). The mixture was stirred at 0° C. for 1 hr. The reaction mixture was purified by prep-HPLC (column: Nano-micro Kromasil C18 100*30 mm 5 um;mobile phase: [water(0.1% TFA)-ACN]; B %: 38%-66%, 10 min) to provide the title compound (12 mg, 15.94 μmol, 6.18% yield, 92% purity) as a white solid.

LCMS (ESI): m/z: [M+H] calcd for C₃₀H₃₅F₆N₄O₈: 693; found 693; RT=1.701 min. ¹H NMR (400 MHz, CHLOROFORM-d) δ 8.21 (d, J=7.28 Hz, 2 H), 7.82 (br d, J=8.38 Hz, 2 H), 7.62-7.85 (m, 1 H), 7.48-7.57 (m, 4 H), 7.23 (d, J=8.60 Hz, 2 H), 5.22-5.34 (m, 2 H), 5.02 (br t, J=10.03 Hz, 1 H), 4.44-4.66 (m, 2 H), 4.19-4.27 (m, 1 H), 3.75 (br s, 1 H), 3.33 (s, 3 H), 3.20 (br s, 1 H), 2.70-2.75 (m, 2 H), 1.93 (br t, J=12.35 Hz, 1 H), 1.79 (br s, 1 H), 1.48-1.63 (m, 2 H), 1.38 (s, 6 H).

### Example 3. Preparation of (S)-N-(1-((2,6-dimethylpyridin-4-yl)oxy)-6-guanidino-2-oxohexan-3-yl)-2-methoxy-2-methylpropanamide (3)

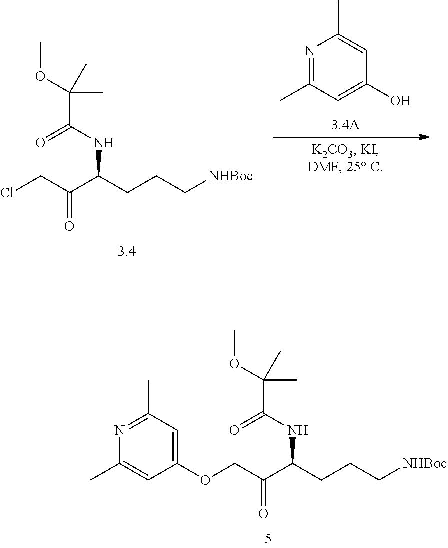

Compound 3.5: To a solution of compound 3.4 (2 g, 5.48 mmol, 1 eq) and compound 3.4A (675.06 mg, 5.48 mmol, 1 eq) in DMF (20 mL) was added K₂CO₃ (2.27 g, 16.44 mmol, 3 eq) and KI (909.94 mg, 5.48 mmol, 1 eq). The mixture was stirred at 25° C. for 1 hr. The residue was diluted with H₂O (20 mL) and extracted with EtOAc (20 mL×3). The combined organic layers were washed with H₂O (20 mL×3), The combined organic layers were washed with brine (50 mL), dried over Na₂SO₄, filtered, and concentrated under reduced pressure to provide a residue which was purified by column chromatography (SiO₂, petroleum ether/ethyl acetate=10:1 to 1:1) to afford compound 3.5 (600 mg, 1.33 mmol, 24.24% yield) as colorless oil.

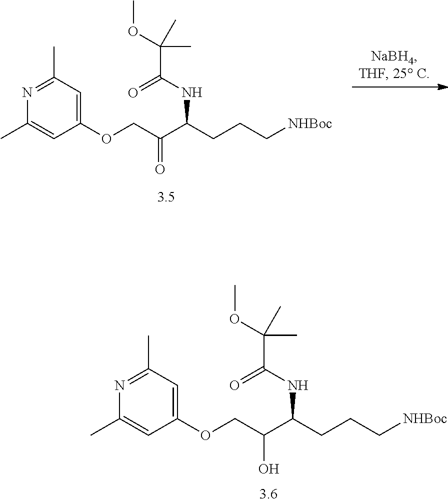 [SMILES: COC(C)(C)C(=O)N[C@@H](CCCNC(=O)OC(C)(C)C)C(=O)COc1cc(C)nc(C)c1 ; "NaBH4,THF, 25° C." ; COC(C)(C)C(=O)N[C@@H](CCCNC(=O)OC(C)(C)C)C(O)COc1cc(C)nc(C)c1]

Compound 3.6: To a solution of compound 3.5 (300 mg, 664.37 μmol, 1 eq) in THF (3 mL) was added NaBH₄ (25.13 mg, 664.37 μmol, 1 eq) at 0° C. The mixture was stirred at 25° C. for 1 hr, filtered, and concentrated under reduced pressure to provide compound 6 (600 mg, crude) as yellow oil.

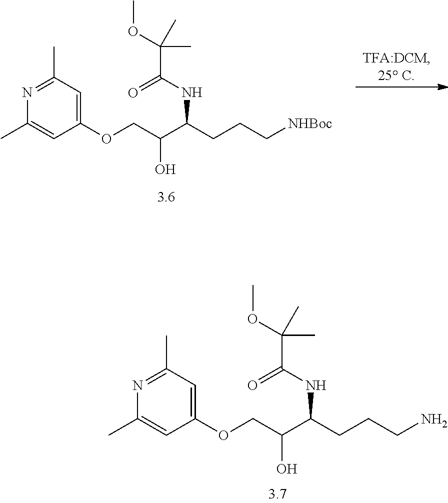 [SMILES: COC(C)(C)C(=O)N[C@@H](CCCNC(=O)OC(C)(C)C)C(O)COc1cc(C)nc(C)c1 ; "TFA:DCM,25° C." ; COC(C)(C)C(=O)N[C@@H](CCCN)C(O)COc1cc(C)nc(C)c1]

Compound 3.7: Compound 3.6 (350 mg, 771.65 μmol, 1 eq) was dissolved in DCM (17.5 mL), and to the resulting mixture was added TFA (5.39 g, 47.27 mmol, 3.50 mL, 61.26 eq) in one portion followed by stirring at 25° C. for 10 mins. The reaction mixture was concentrated under reduced pressure to provide compound 3.7 (300 mg, 641.74 μmol, 83.16% yield, TFA) as yellow oil. LCMS (ESI): m/z: [M+H] calcd for C₁₈H₃₂N₃O₄.C₂HF₃O₂: 354; found 354; RT=0.793 min.

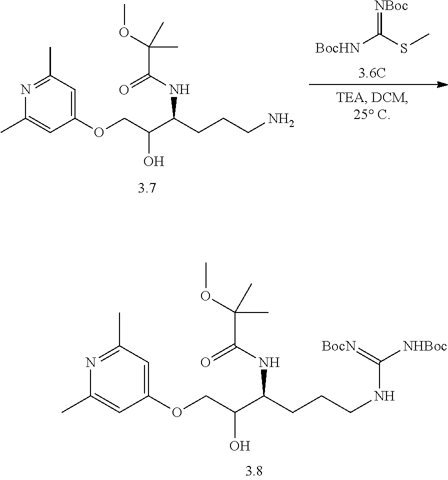

Compound 3.8: To a mixture of compound 3.7 (160 mg, 342.26 μmol, 1 eq, TFA) and compound 3.6C (198.77 mg, 684.52 μmol, 2 eq) in DCM (5 mL) was added TEA (103.90 mg, 1.03 mmol, 142.92 μL, 3 eq) in one portion at 25° C. under N₂. The mixture was stirred at 25° C. for 30 mins. The reaction mixture was concentrated under reduced pressure to provide a residue which was purified by column chromatography (SiO₂, ethyl acetate) to afford compound 3.8 (0.2 g, 335.72 μmol, 98.09% yield) as yellow oil.

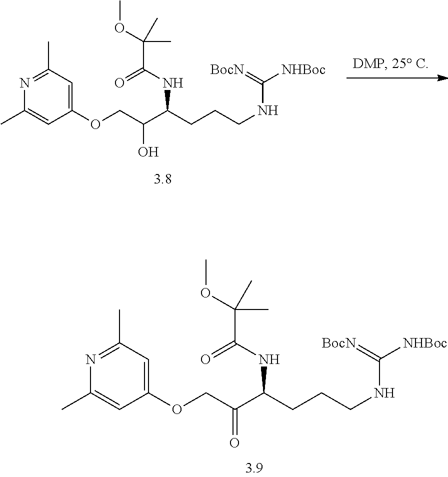

Compound 3.9: To a mixture of compound 3.8 (200 mg, 335.72 μmol, 1 eq) in DCM (5 mL) was added Dess-Martin periodinane (284.79 mg, 671.45 μmol, 207.88 μL, 2 eq) in one portion at 25° C. under N₂. The mixture was stirred at 25° C. for 30 mins. The reaction mixture was filtered and concentrated under reduced pressure to provide a residue which was purified by column chromatography (SiO₂, petroleum ether/ethyl acetate=1:1) to afford compound 9 (100 mg, 168.43 μmol, 50.17% yield) as yellow oil.

¹FINMR (400 MHz, CHLOROFORM-d) δ 6.52 (s, 2H), 4.92-4.79 (m, 3H), 3.54-3.43 (m, 1H), 3.46-3.40 (m, 1H), 3.31 (s, 3H), 2.49 (s, 6H), 1.98 (br m, 1H), 1.72-1.64 (m, 3H), 1.49 (d, J=1.8 Hz, 20H), 1.40-1.38 (d, J=11.4 Hz, 6H). LCMS (ESI): m/z: [M+H] calcd for C₂₉H₄₈O₈N₅: 594; found 594; RT=1.155 min.

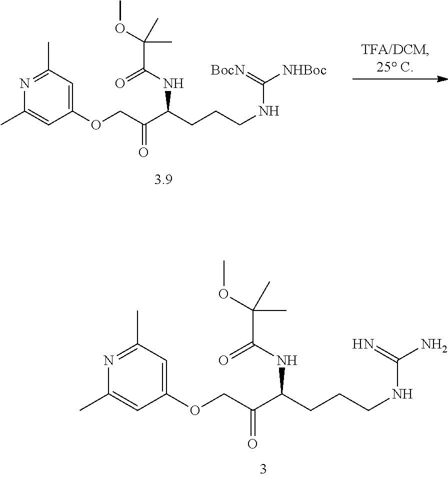

Compound 3: To a solution of compound 3.9 (50 mg, 84.22 μmol, 1 eq) in DCM (5 mL) was added TFA (1.54 g, 13.51 mmol, 1 mL, 160.37 eq) in one portion at 25° C. under N₂. The mixture was stirred at 25° C. for 1 hour. The reaction mixture was concentrated under reduced pressure to provide the title product (35 mg, 67.53 μmol, 80.19% yield, 97.92% purity, TFA) as yellow oil.

¹H NMR (400 MHz, METHANOL-d4) δ 7.23-7.16 (m, 2H), 5.36-5.23 (m, 2H), 4.47-4.50 (m, 1H), 3.32 (s, 3H), 3.25-3.18 (m, 2H), 2.63 (s, 6H), 2.10-1.95 (m, 1H), 1.82-1.52 (m, 3H), 1.40 (d, J=5.7 Hz, 6H). LCMS (ESI): m/z: [M+H] calcd for C₁₉H₃₁N₅O₄.C₂HF₃O₂: 394; found 394; RT=2.245 min.

### Example 4. Preparation of (S)-N-(6-guanidino-1-((2-methylpyrimidin-5-yl)oxy)-2-oxohexan-3-yl)-2-methoxy-2-methylpropanamide (4)

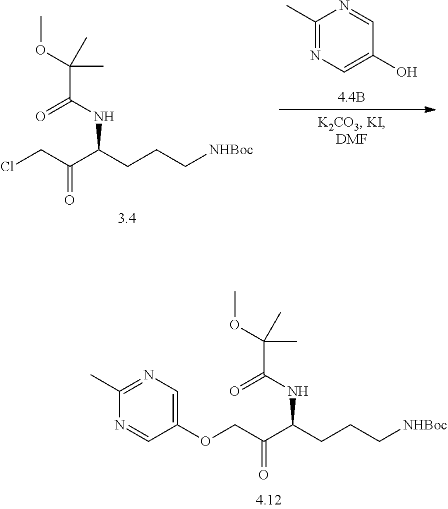

Compound 4.12: To a solution of compound 3.4 (800 mg, 2.19 mmol, 1 eq) and compound 4.4B (241.44 mg, 2.19 mmol, 1 eq) in DMF (8 mL) was added K₂CO₃ (909.09 mg, 6.58 mmol, 3 eq) and KI (363.98 mg, 2.19 mmol, 1 eq). The mixture was stirred at 25° C. for 1 hr. The residue was diluted with H₂O (5 mL) and extracted with EtOAc (5 mL×3). The combined organic layers were washed with H₂O (5 mL×3). The combined organic layers were washed with brine (10 mL), dried over Na₂SO₄, filtered, and concentrated under reduced pressure to provide a residue which was purified by column chromatography (SiO₂, petroleum ether/ethyl acetate=10:1 to 1:1) to afford compound 4.12 (380 mg, 866.56 μmol, 39.52% yield) as yellow oil. LCMS (ESI): m/z: [M+H] calcd for C₂₁H₃₅O₄N₆: 439; found 439, RT=1.036min.

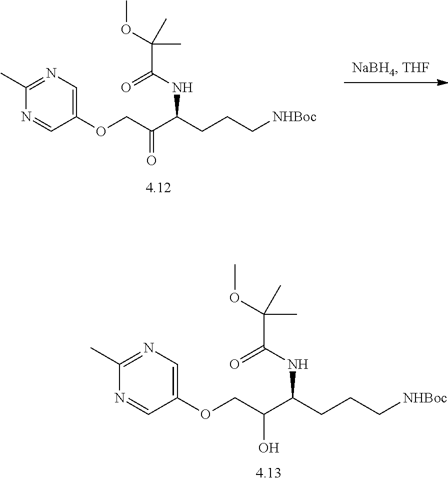

Compound 4.13: To a solution of compound 4.12 (380 mg, 866.56 μmol, 1 eq) in THF (4 mL) was added NaBH₄ (65.57 mg, 1.73 mmol, 2eq) at 0° C. The mixture was stirred at 25° C. for 1 hr. The residue was diluted with H₂O (5 mL) and extracted with EtOAc (5 mL×3). The combined organic layers were washed with H₂O (5 mL×3). The combined organic layers were washed with brine (10 mL), dried over Na₂SO₄, filtered, and concentrated under reduced pressure to afford compound 4.13 (400 mg, crude) as yellow oil. LCMS (ESI): m/z: [M+H] calcd for C₂₁H₃₇O₄N₆: 441; found 441, RT=1.127min.

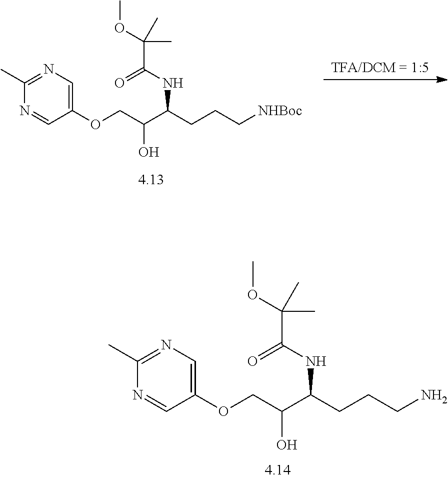

Compound 4.14: To a solution of compound 4.13 (400 mg, 907.99 μmol, 1 eq) in DCM (5 mL) was added TFA (1.53 g, 13.44 mmol, 995.43 μL, 14.81 eq). The mixture was stirred at 25° C. for 0.5 hr. The residue was diluted with H₂O (5 mL) and extracted with DCM (5 mL×3). The combined organic layers were washed with H₂O (5 mL×3). The combined organic layers were washed with brine (10 mL), dried over Na₂SO₄, filtered and concentrated under reduced pressure to afford compound 14.4 (780 mg, crude) as yellow solid.

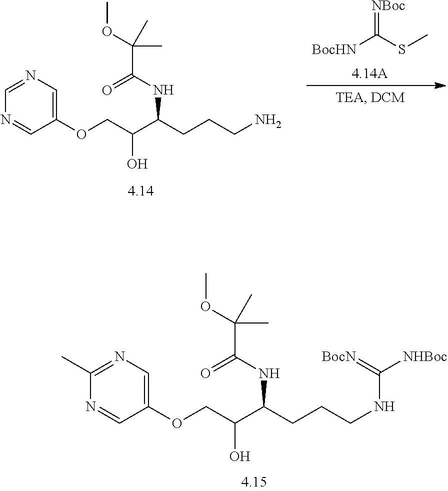

Compound 4.15: To a solution of compound 4.14 (660 mg, 1.94 mmol, 1 eq) and compound 4.14A (1.13 g, 3.88 mmol, 2 eq) in DCM (7 mL) was added TEA (588.56 mg, 5.82 mmol, 809.58 uL, 3 eq). The mixture was stirred at 25° C. for 10 hr. The residue was diluted with H₂O (5 mL) and extracted with DCM (5 mL×3). The combined organic layers were washed with H₂O (5 mL×3). The combined organic layers were washed with brine (10 mL), dried over Na₂SO₄, filtered, and concentrated under reduced pressure to provide a residue which was purified by column chromatography (SiO₂, petroleum ether/ethyl acetate=10:1 to 1:1) to afford compound 4.15 (440 mg, 755.12 μmol, 38.92% yield) as colorless oil. LCMS (ESI): m/z: [M+H] calcd for C₂₁H₄₇O₈N₆: 583; found 583, RT=1.071min.

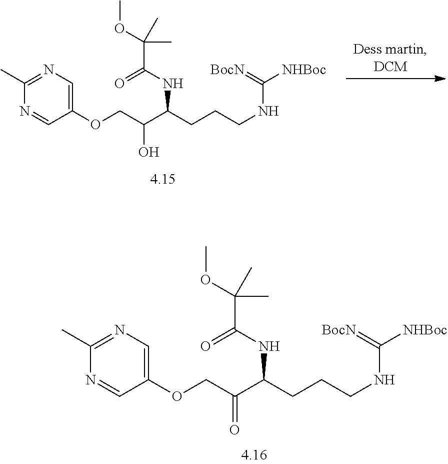

Compound 4.16: To a solution of compound 4.15 (438.48 mg, 752.52 μmol, 1 eq) in DCM (5 mL) was added Dess-Martin periodinane (638.35 mg, 1.51 mmol, 465.95 uL, 2 eq). The mixture was stirred at 25° C. for 10 hr, filtered, and concentrated under reduced pressure to provide a residue which was purified by prep-HPLC (column: Waters Xbridge 150×25 5u; mobile phase: [water (0.04% NH₃H₂O+10 mM NH₄HCO₃)-ACN]; B %: 35%-70%, 10 min) to afford compound 4.16 (110 mg, 189.44 μmol, 25.17% yield) as colorless oil. LCMS (ESI): m/z: [M+H] calcd for C₂₇H₄₅O₈N₆: 581; found 581, RT=1.219 min.

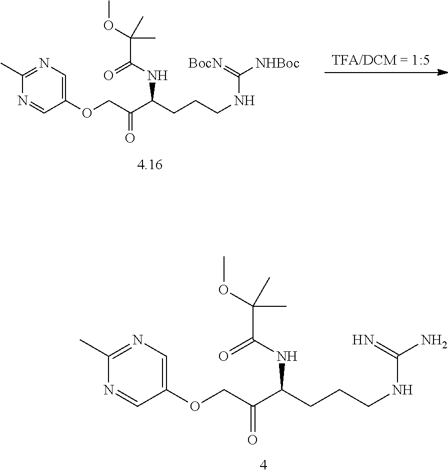

Compound 4: To a solution of compound 4.16 (30.00 mg, 51.66 μmol, 1 eq) in DCM (5 mL) was added TFA (1 mL). The mixture was stirred at 25° C. for 2 hr. Filtered and concentrated under reduced pressure to afford the title compound (20 mg, 39.96 μmol, 77.35% yield, 98.8% purity, TFA) as a colorless oil.

LCMS (ESI): m/z: [M+H] calcd for C₁₇H₂₉O₄N₆: 381; found 381, RT=1.949min. ¹H NMR (400 MHz, METHANOL-d4) δ 8.22-8.42 (m, 2 H), 4.89-5.07 (m, 2 H), 4.50 (m, 1 H), 3.23 (s, 3 H), 3.11-3.16 (m, 2 H), 2.54 (s, 3 H), 1.89-2.02 (m, 1 H), 1.48-1.92 (m, 3 H), 1.26-1.30 (t, 6 H).

### Example 5. Preparation of (S)-N-(6-guanidino-1-(isoxazol-3-yloxy)-2-oxohexan-3-yl)-2-methoxy-2-methylpropanamide (5)

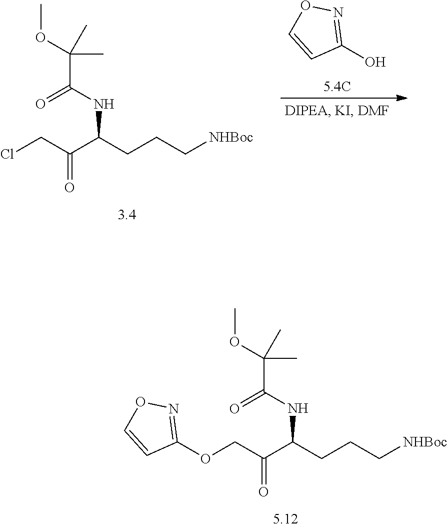

Compound 5.12: To a solution of compound 4 (1 g, 2.74 mmol, 1 eq), isoxazol-3-ol (186.51 mg, 2.19 mmol, 0.8 eq) in DMF (10 mL) was added DIPEA (708.45 mg, 5.48 mmol, 954.78 μL, 2 eq) and KI (454.97 mg, 2.74 mmol, 1 eq). The mixture was stirred at 25° C. for 12 hr. The reaction mixture was quenched by addition of H₂O (20 mL), and then extracted with EtOAc (20 mL×3). The combined organic layers were washed with H₂O (20 mL×3), and saturated brine (40 mL), dried over Na₂SO₄, filtered, and concentrated under reduced pressure to provide a residue which was purified by MPLC (SiO₂, petroleum ether/ethyl acetate=10/1 to 1:1) to afford compound 5.12 (380 mg, 919.06 μμmol, 33.53% yield) as a white solid.

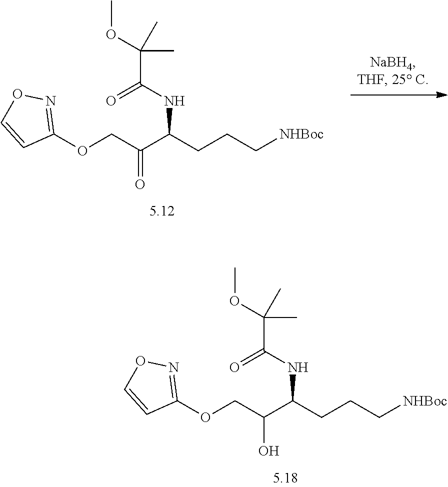 [SMILES: COC(C)(C)C(=O)N[C@@H](CCCNC(=O)OC(C)(C)C)C(=O)COc1ccon1 ; COC(C)(C)C(=O)N[C@@H](CCCNC(=O)OC(C)(C)C)C(O)COc1ccon1]

Compound 5.18: To a solution of compound 5.12 (380 mg, 919.06 μmol, 1 eq) in THF (5 mL) was added NaBH₄ (69.54 mg, 1.84 mmol, 2 eq). The mixture was stirred at 25° C. for 0.5 h. The reaction mixture was quenched by addition of H₂O (5 mL), and then extracted with EtOAc (5 mL×3). The combined organic layers were washed with H₂O (5 mL×3), and saturated brine (5 mL), dried over Na₂SO₄, filtered, and concentrated under reduced pressure to afford compound 5.18 (700 mg, crude) as a yellow oil.

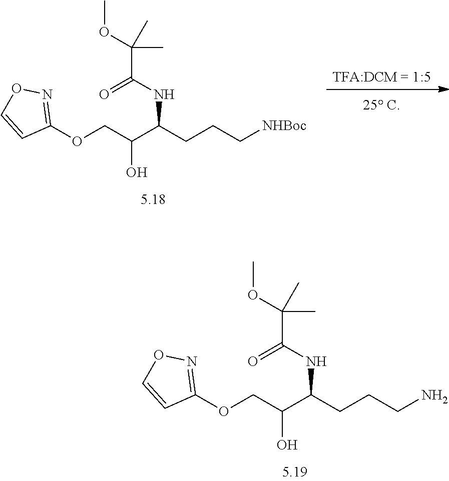

Compound 19: To a solution of TFA (1.54 g, 13.51 mmol, 1 mL, 8.02 eq) in DCM (5 mL) was added compound 5.18 (700 mg, 1.68 mmol, 1 eq). The mixture was stirred at 25° C. for 1 hr. The reaction mixture was concentrated under reduced pressure to afford compound 5.19 (700 mg, crude) as a yellow oil.

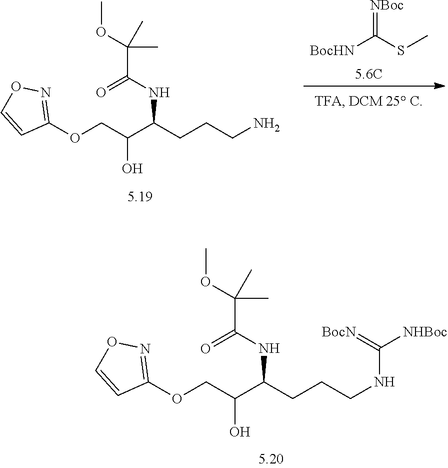

Compound 20: To a solution of compound 5.19 (700.00 mg, 2.22 mmol, 1 eq) and compound 5.6C (1.29 g, 4.44 mmol, 2 eq) in DCM (10 mL) was added TEA (673.82 mg, 6.66 mmol, 926.85 μL, 3 eq). The mixture was stirred at 25° C. for 12 hr. The reaction mixture was concentrated under reduced pressure provide a residue which was purified by column chromatography (SiO₂, petroleum ether/ethyl acetate=10/1 to 1:2) to afford compound 5.20 (260 mg, 373.00 μmol, 16.80% yield, 80% purity) as a yellow oil.

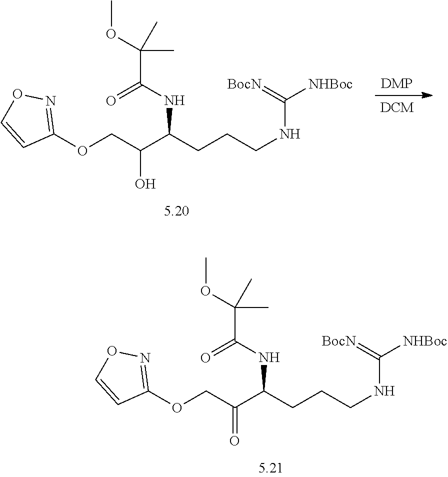

Compound 21: To a solution of compound 5.20 (260 mg, 466.25 μmol, 1 eq) in DCM (5 mL) was added Dess-Martin periodinane (395.52 mg, 932.51 μmol, 288.70 μL, 2 eq). The mixture was stirred at 5° C. for 12 hr. The reaction mixture was quenched by addition of H₂O (5 mL), and then extracted with DCM (5 mL×3). The combined organic layers were washed with Na₂SO₃ (5 mL×3) and saturated brine (5 mL), dried over Na₂SO₄, filtered, and concentrated under reduced pressure to provide a residue which was purified by prep-HPLC (column: Waters Xbridge 150*25 5u; mobile phase: [water (0.04% NH₃H₂O+10 mM NH₄HCO₃)-ACN]; B %: 45%-65%, 10 min) to afford compound 5.21 (60 mg, 107.99 μmol, 23.16% yield) as a yellow oil.

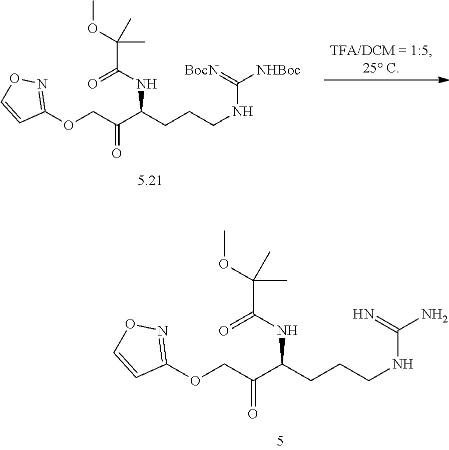

Compound 5: To a solution of compound 5.21 (60 mg, 107.99 μmol, 1 eq) in DCM (5 mL) was added TFA (1.54 g, 13.51 mmol, 1 mL, 125.07 eq). The mixture was stirred at 25° C. for 2 hr. The reaction mixture was concentrated under reduced pressure to provide the title compound (35 mg, crude) as a yellow oil.

LCMS (ESI): m/z: [M+H] calcd for C₁₅H₂₆N₅O₅: 356; found 356; RT=1.965 min. ¹H NMR (400 MHz, METHANOL-d4) δ 8.38 (d, J=2.8 Hz, 1 H), 6.16 (d, J=2.8Hz, 1 H), 5.02-5.05 (m, 1 H), 4.59 (dd, J =9.15, 4.52 Hz, 1 H), 3.29 (br s, 3 H), 3.14-3.25 (m, 2 H), 1.98-2.09 (m, 1 H), 1.49-1.73 (m, 3 H), 1.39 (s, 6 H).

### Example 6. Preparation of (S)-N-(6-guanidino-1-((1,1,1,3,3,3-hexafluoropropan-2-yl)oxy)-2-oxohexan-3-yl)nicotinamide (6)

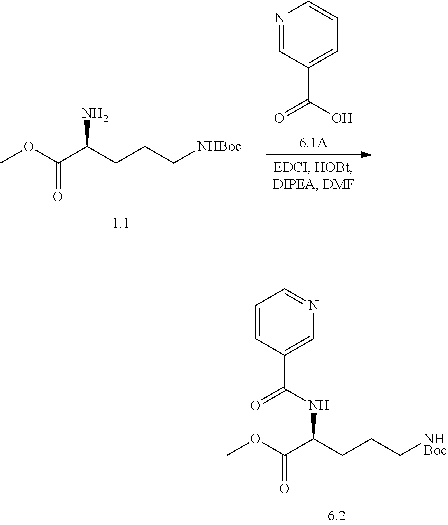

Compound 6.2: To a solution of compound 6.1A (3.48 g, 28.29 mmol, 2.37 mL, 1 eq) in DMF (80 mL) was added HOBt (4.97 g, 36.78 mmol, 1.3 eq) and EDCI (7.05 g, 36.78 mmol, 1.3 eq) at 0° C. for 1 hr. Then compound 1.1 (8 g, 28.29 mmol, 1 eq, HCl) and DIPEA (10.97 g, 84.88 mmol, 14.78 mL, 3 eq) were added to the mixture at 0° C. for 1 hr. The mixture was diluted with H₂O (100 mL) and extracted with EtOAc (100 mL×3). The combined organic layers were washed with H₂O (100 mL×3). The combined organic layers were washed with brine (300 mL×1), dried over Na₂SO₄, filtered, and concentrated under reduced pressure to provide a residue which was purified by column chromatography (SiO₂, petroleum ether/ethyl acetate=10:1 to 1:1) to afford compound 6.2 (8.83 g, 25.13mmol, 88.82% yield) as a colorless oil.

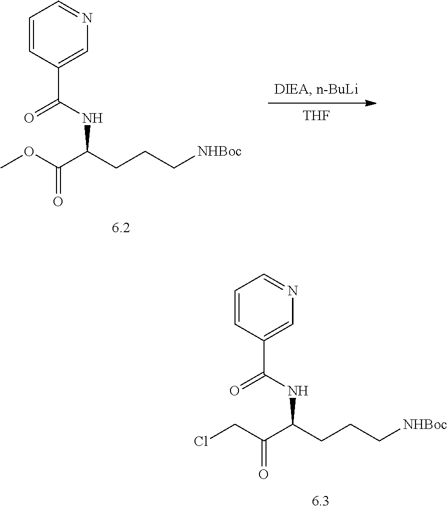 [SMILES: COC(=O)[C@H](CCCNC(=O)OC(C)(C)C)NC(=O)c1cccnc1 ; "DIEA, n-BuLi" ; "THF" ; CC(C)(C)OC(=O)NCCC[C@H](NC(=O)c1cccnc1)C(=O)CCl]

Compound 6.3: To a solution of DIEA (13.99 g, 138.21 mmol, 19.53 mL, 5.5 eq) in THF (40mL) was added n-BuLi (2.5 M, 55.28 mL, 5.5 eq) at 0° C., the mixture was stirred at 0° C. for 30 mins. The mixture was added to a solution of chloro(iodo)methane (24.38 g, 138.21 mmol, 10.03 mL, 5.5 eq) and compound 6.2 (8.83 g, 25.13 mmol, 1 eq) in THF (40 mL) at −78° C. The mixture was stirred at −78° C. for 3 h prior to dilution with H₂O (50 mL) and extracted with EtOAc (50 mL×3). The combined organic layers were washed with H₂O (50 mL×3). The combined organic layers were washed with brine (100 mL), dried over Na₂SO₄, filtered and concentrated under reduced pressure to provide a residue which was purified by column chromatography (SiO₂, petroleum ether/ethyl acetate=10:1 to 1:1) to afford compound 6.3 (4.86 g, 13.14 mmol, 52.29% yield) as a yellow oil. LCMS (ESI): m/z: [M+H] calcd for C₁₇H₂₅O₃ClN₄: 370; found 370, RT=0.897 min.

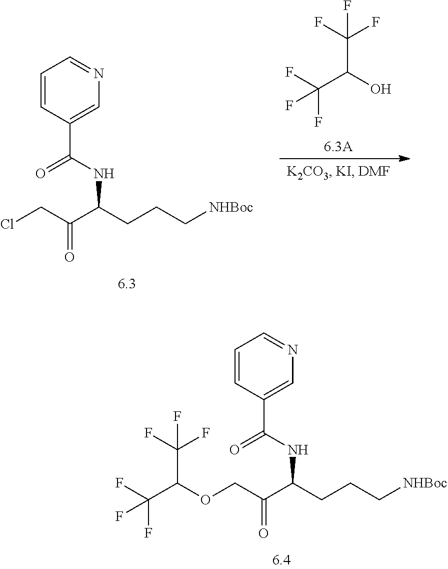

Compound 6.4: To a solution of compound 6.3 (4.86 g, 13.14 mmol, 1 eq) and compound 6.3A (2.21 g, 13.14 mmol, 1 eq) in DMF (50 mL) was added K₂CO₃ (5.45 g, 39.42 mmol, 3 eq) and KI (2.18 g, 13.14 mmol, 1 eq). The mixture was stirred at 25° C. for 1 hr. The residue was diluted with H₂O (50 mL) and extracted with EtOAc (50 mL×3). The combined organic layers were washed with H₂O (100 mL×3). The combined organic layers were washed with brine (200 mL), dried over Na₂SO₄, filtered, and concentrated under reduced pressure to provide a residue which was purified by column chromatography (SiO₂, petroleum ether/ethyl acetate=10:1 to 1:1) to afford compound 6.4 (4.5 g, 8.97 mmol, 68.30% yield) as a yellow oil. LCMS (ESI): m/z: [M+H] calcd for C₂₀H₂₆O₅F₆N₃: 502; found 502, RT=1.113 min.

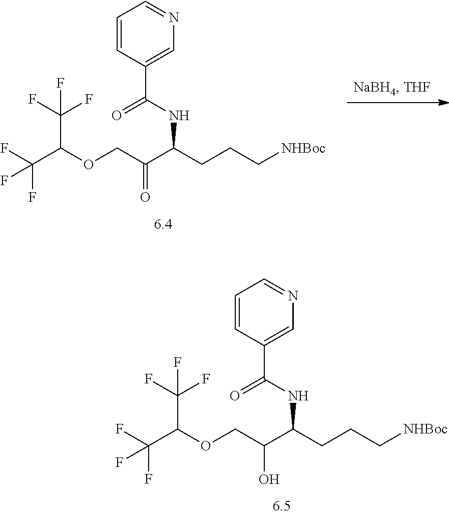

Compound 6.5: To a solution of compound 6.4 (4 g, 7.98 mmol, 1 eq) in THF (40 mL) was added NaBH₄ (603.61 mg, 15.95 mmol, 2eq). The mixture was stirred at 25° C. for 1 hr. The residue was diluted with H₂O (30 mL) and extracted with EtOAc (20 mL×3). The combined organic layers were washed with brine (50 mL), dried over Na₂SO₄, filtered, and concentrated under reduced pressure to afford compound 6.5 (1 g, crude) as a yellow oil.

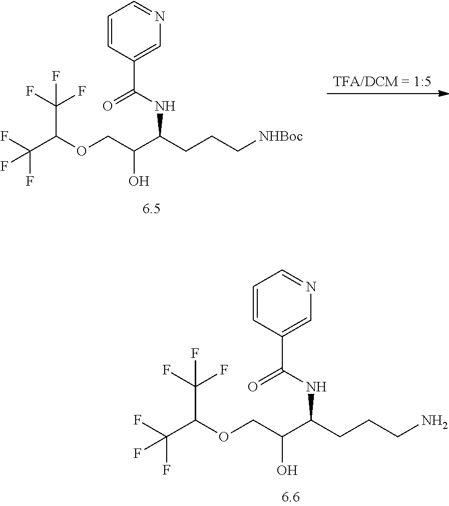

Compound 6.6: To a solution of compound 6.5 (800 mg, 1.59 mmol, 1 eq) in DCM (5 mL) was added TFA (1.54 g, 13.51 mmol, 1 mL, 8.50 eq). The mixture was stirred at 25° C. for 1 hr. Then the mixture was filtered and concentrated under reduced pressure to afford compound 6.6 (1.2 g, crude) as a colorless oil.

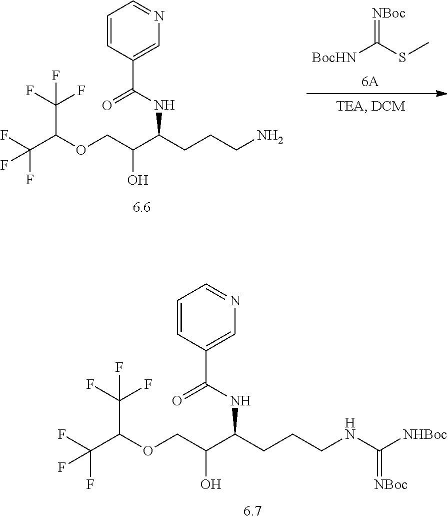

Compound 6.7: To a solution of compound 6 (1.12 g, 2.78 mmol, 1 eq) and compound 6A (1.62 g, 5.56 mmol, 2 eq) in DCM (10 mL) was added TEA (844.20 mg, 8.34 mmol, 1.16 mL, 3 eq). The mixture was stirred at 25° C. for 10 hr. The mixture was diluted with H₂O (5 mL) and extracted with DCM (5 mL×3). The combined organic layers were washed with H₂O (5 mL×3). The combined organic layers were washed with brine (10 mL), dried over Na₂SO₄, filtered, and concentrated under reduced pressure to provide a residue which was purified by prep-HPLC (column: Phenomenex Luna C18 200*40 mm*10 um; mobile phase: [water (0.1% TFA)-ACN]; B %: 35%-55%, 10 min) to afford compound 6.7 (200 mg, 309.79 μmol, 11.14% yield) as a colorless oil. LCMS (ESI): m/z: [M+H] calcd for C₂₆H₃₈O₇F₆N₅: 646; found 646, RT=1.123min.

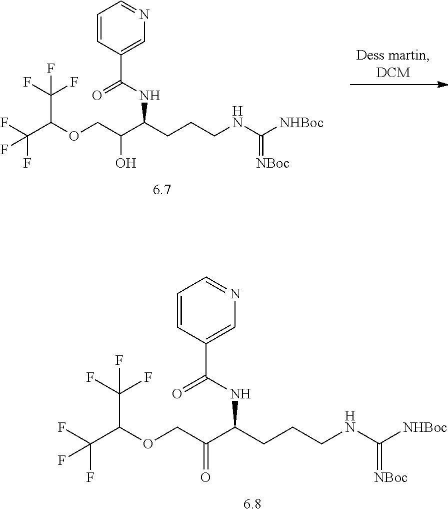

Compound 6.8: To a solution of compound 6.7 (200 mg, 309.79 μmol, 1 eq) in DCM (3 mL) was added Dess-Martin periodinane (262.79 mg, 619.59 μmol, 191.82 uL, 2 eq). The mixture was stirred at 25° C. for 1 hr, diluted with H₂O (3 mL) and extracted with EtOAc (3 mL×3). The combined organic layers were washed with H₂O (3 mL×3). The combined organic layers were washed with brine (10 mL), dried over Na₂SO₄, filtered, and concentrated under reduced pressure to provide compound 6.8 (100 mg, 155.38 μmol, 50.16% yield) as colorless oil.

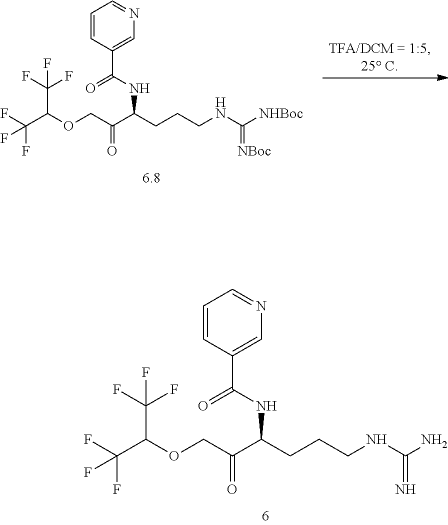

Compound 6: To a solution of compound 6.8 (99.69 mg, 154.90 μmol, 1 eq) in DCM (5 mL) was added TFA (1.54 g, 13.51 mmol, 1 mL, 87.19 eq). The mixture was stirred at 25° C. for 1 hr. Then the mixture was filtered and concentrated under reduced pressure to provide a residue which was purified by prep-HPLC (column: Phenomenex Synergi C18 100*30 mm*4 um;mobile phase: [water (0.1% TFA)-ACN]; B %: 20%-40%, 10 min) to afford the title compound, (10 mg, 17.71 μmol, 11.43% yield, 98.7% purity, TFA) as a colorless oil.

LCMS (ESI): m/z: [M+H] calcd for C₁₆H₂₀O₃F₆N₅: 444; found 444, RT=2.791 min. ¹H NMR (400 MHz, METHANOL-d4) δ 9.10 (br s, 1 H), 8.81 (br s, 1 H), 8.43 (d, J=7.95 Hz, 1 H), 7.72 (d, J=4.52 Hz, 1 H), 5.03-5.13 (m, 1 H), 4.77-4.86 (m, 3 H), 3.22-3.30 (m, 2 H), 1.65-2.12 (m, 4 H).

### Example 7. Preparation of (S)-N-(6-(3-cyanoguanidino)-1-((1,1,1,3,3,3-hexafluoropropan-2-yl)oxy)-2-oxohexan-3-yl)-2-methoxy-2-methylpropanamide (7)

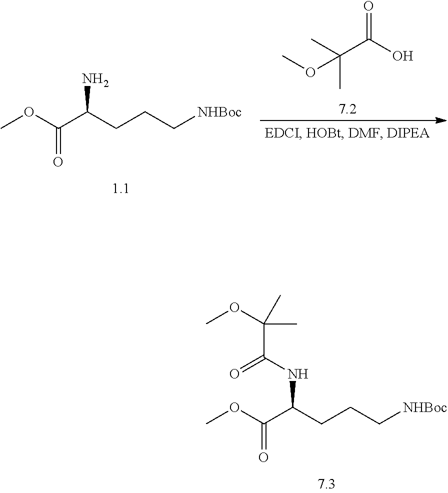

Compound 7.3: To a mixture of compound 7.2 (12.53 g, 106.10 mmol, 1 eq) and HOBt (15.77 g, 116.71 mmol, 1.1 eq) in DMF (300 mL) was added EDCI (22.37 g, 116.71 mmol, 1.1 eq) in one portion at 0° C. under N₂. The mixture was stirred at 0° C. for 60 mins, then the mixture was added compound 1 (30 g, 106.10 mmol, 1 eq, HCl) and DIPEA (41.14 g, 318.29 mmol, 55.44 mL, 3 eq), then the mixture was stirred at 0° C. for 30 mins. The reaction mixture was diluted with H₂O (100 mL) and extracted with EtOAc (100 mL×3). The combined organic layers were washed with H₂O (200 mL×3) and brine (300 mL), dried over Na₂SO₄, filtered and concentrated under reduced pressure to provide a residue which was purified by column chromatography (SiO₂, petroleum ether/ethyl acetate=10:1 to 1:1) to afford compound 3 (43 g, crude) as colorless oil. LCMS (ESI): m/z: [M+H] calcd for C₁₆H₃₁O₆N₂: 347; found: 347, RT=1.061min.

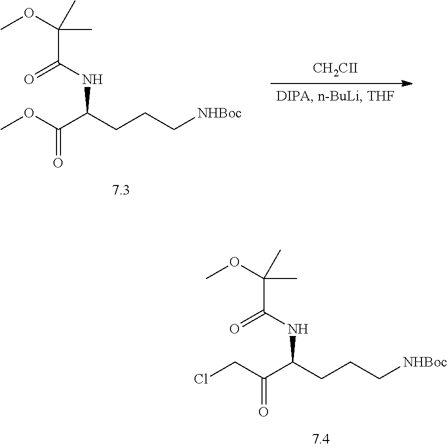

Compound 7.4: To a solution of DIPA (20.89 g, 206.40 mmol, 29.17 mL, 5.5 eq) in THF (100 mL) was added n-BuLi (2.5 M, 82.56 mL, 5.5eq) at 0° C., the mixture was stirred at 0° C. for 30 mins. The mixture was added a solution of chloro (iodo) methane (36.41 g, 206.40 mmol, 14.98 mL, 5.5 eq) and compound 7.3 (13 g, 37.53 mmol, 1 eq) in THF (100 mL) at −78° C. The mixture was stirred at −78° C. for 3 h. The residue was diluted with H₁₂O (100 mL) and extracted with EtOAc (100 mL×3). The combined organic layers were washed with H₂O (100 mL×3). The combined organic layers were washed with brine (200 mL×1), dried over Na₂SO₄, filtered, and concentrated under reduced pressure to provide a residue which was purified by column chromatography (SiO₂, petroleum ether/ethyl acetate=10:1 to 1:1) to afford compound 7.4 (13.71 g, crude) as yellow oil.

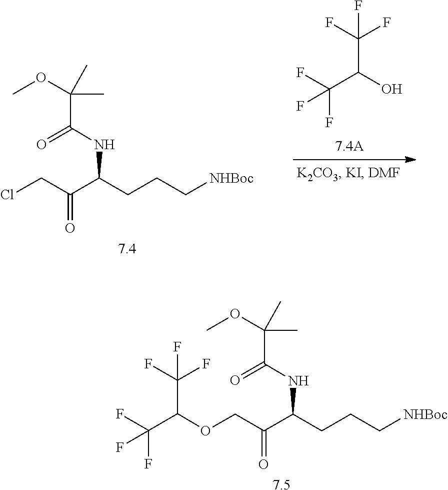

Compound 7.5: To a solution of compound 7.4 (13.71 g, 37.57 mmol, 1 eq) and compound 7.4A (6.31 g, 37.57 mmol, 1 eq) in DMF (100 mL) was added K₂CO₃ (15.58 g, 112.71 mmol, 3 eq) and KI (6.24 g, 37.57 mmol, 1 eq). The mixture was stirred at 25° C. for 1 hr. The residue was diluted with H₂O (100 mL) and extracted with EtOAc (100 mL×3). The combined organic layers were washed with H₂O (100 mL×3). The combined organic layers were washed with brine (200 mL), dried over Na₂SO₄, filtered, and concentrated under reduced pressure to provide a residue which was purified by column chromatography (SiO₂, petroleum ether/ethyl acetate=10:1 to1:1) to afford compound 7.5 (5.5 g, 11.08 mmol, 29.49% yield) as yellow oil.

¹H NMR (400 MHz, CHLOROFORM-d) δ 1.38-1.42 (m, 6 H), 1.46-1.50 (m, 9 H), 1.55-1.58 (m, 3 H), 1.91-1.96 (m, 1 H), 3.14-3.17 (m, 2 H), 3.32 (s, 3 H), 4.33-4.36 (m, J=11.31, 5.59 Hz, 1 H), 4.62 (br d, J=3.06 Hz, 2 H), 4.69-4.74 (m, 1 H), 7.19-7.21 (br d, J=7.34 Hz, 1 H).

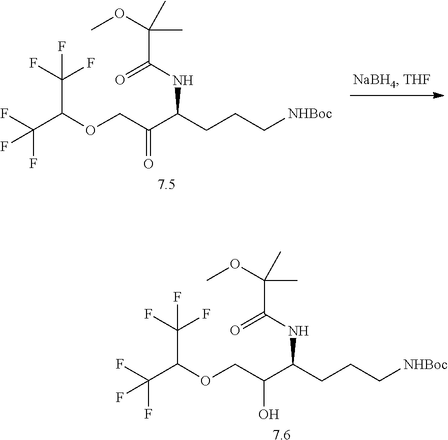

Compound 7.6: To a solution of compound 7.5 (5.5 g, 11.08 mmol, 1 eq) in THF (60 mL) was added NaBH₄ (838.28 mg, 22.16 mmol, 2 eq) at 0° C. The mixture was stirred at 25° C. for 1 hr. The residue was diluted with H₂O (50 mL) and extracted with EtOAc (50 mL×3). The combined organic layers were washed with H₂O (100 mL×3). The combined organic layers were washed with brine (200 mL), dried over Na₂SO₄, filtered, and concentrated under reduced pressure to afford compound 7.6 (6 g, crude) as colorless oil.

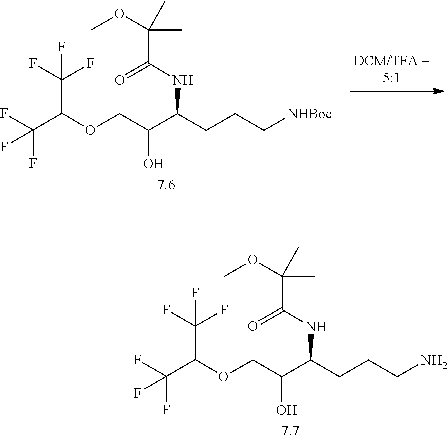

Compound 7.7: To a solution of compound 7.6 (6 g, 12.04 mmol, 1 eq) in DCM (50 mL) was added TFA (15.40 g, 135.06 mmol, 10 mL, 11.22 eq). The mixture was stirred at 25° C. for 1 hr, filtered, and concentrated under reduced pressure to provide compound 7.7 (9.7 g, crude) as colorless oil.

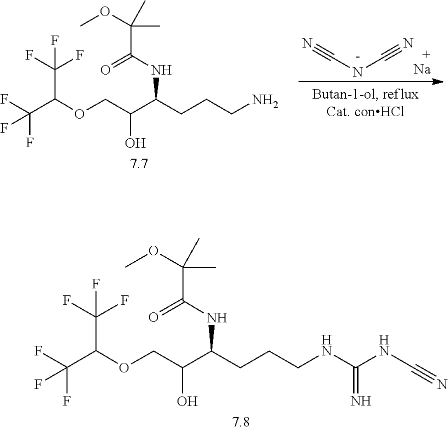

Compound 7.8: To a solution of compound 7.7 (300 mg, 753.12 μmol, 1 eq) and (dicyanoamino) sodium (73.76 mg, 828.44 μmol, 1.1 eq) in n-BuOH (3 mL) was added DIPEA (97.34 mg, 753.12 μmol, 131.18 μL, 1 eq). The mixture was stirred at 120° C. for 0.5 hr. The residue was diluted with H₂O (3 mL) and extracted with EtOAc (3 mL×3). The combined organic layers were washed with H₂O (3 mL×3). The combined organic layers were washed with brine (9 mL), dried over Na₂SO₄, filtered, and concentrated under reduced pressure to afford compound 7.8 (840 mg, crude) as colorless oil.

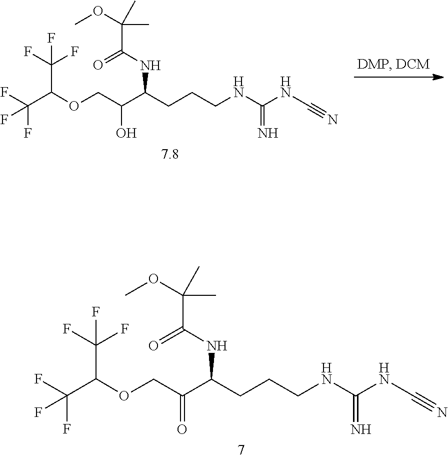 [SMILES: COC(C)(C)C(=O)N[C@@H](CCCNC(=N)NC#N)C(O)COC(C(F)(F)F)C(F)(F)F ; "7.8" ; COC(C)(C)C(=O)N[C@@H](CCCNC(=N)NC#N)C(=O)COC(C(F)(F)F)C(F)(F)F ; "7"]

Compound 7: To a solution of compound 7.8 (840 mg, 1.80 mmol, 1 eq) in DCM (1 mL) was added Dess-Martin periodinane (1.53 g, 3.61 mmol, 1.12 mL, 2eq). The mixture was stirred at 25° C. for 0.5 hr. Filtered and concentrated under reduced pressure to provide a residue which was purified by prep-HPLC (column: Nano-micro Kromasil C18 100×30 mm Sum; mobile phase: [water(0.1% TFA)-ACN]; B %: 30%-52%,10 min) to provide the title compound, (10 mg, 18.56 μmol, 1.03% yield, 86% purity) as a white solid.

LCMS (ESI): m/z: [M+H] calcd for C₁₆H₂₄O₄F₆N₅: 464; found 464, RT=2.845min. ¹H NMR (400 MHz, CHLOROFORM-d) δ 4.98-5.04 (m 1 H), 4.44-4.64 (m, 2 H), 4.26 (m, 1 H), 3.32 (s, 3 H), 3.14-3.16 (m, 2 H), 1.88-1.97 (m, 1 H), 1.57-1.66 (m, 2 H), 1.36-1.37 (d, J=3.42 Hz, 6 H).

### Example 8. Preparation of (5)-N-(1-((1,1,1,3,3,3-hexafluoropropan-2-yl)oxy)-6-(3-(4-nitrobenzylformyl)guanidino)-2-oxohexan-3-yl)-2-methoxy-2-methylpropanamide (8)

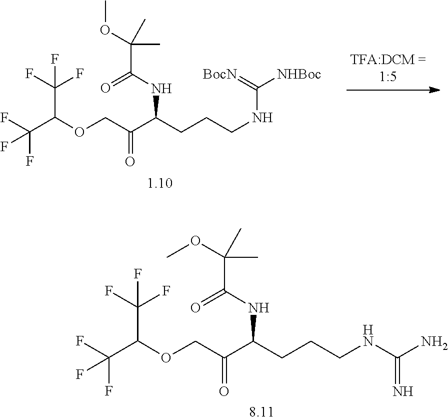

Compound 8.11: To a solution of compound 1.10 (3.5 g, 5.48 mmol, 1 eq) in DCM (50 mL) was added TFA (15.40 g, 135.06 mmol, 10 mL, 24.64 eq). The mixture was stirred at 25° C. for 1 hr, filtered, and concentrated under reduced pressure to provide a residue which was purified by prep-HPLC (column: YMC-Exphere C18 10 um 300*50 mm 12 nm; mobile phase: [water (10 mM NH₄HCO₃)-ACN]; 20 min) to afford compound 8.11 (1.8 g, 4.11 mmol, 74.92% yield) as colorless oil.

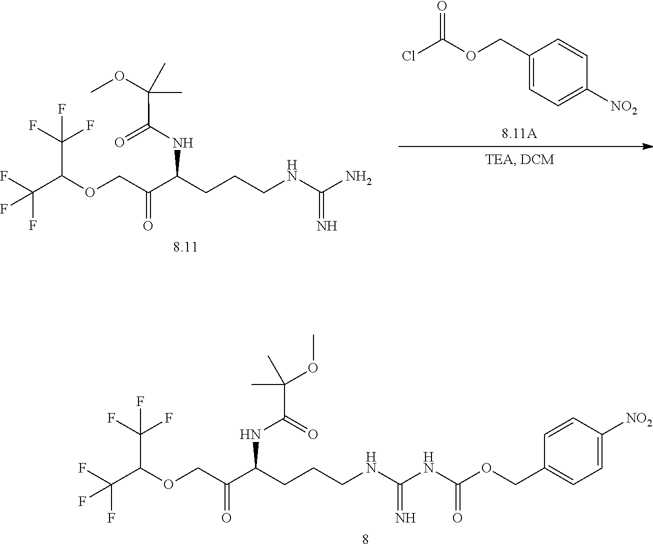

Compound 8: To a solution of compound 8.11A (24.59 mg, 114.06 umol, 0.5 eq) in DCM (1 mL) was added TEA (46.17 mg, 456.24 μmol, 63.50 μL, 2 eq) and compound 8.11 (100 mg, 228.12 umol, 1 eq). The mixture was stirred at 0° C. for 0.5 hr. Filtered and concentrated under reduced pressure to provide a residue which was purified by prep-HPLC (column: Waters Xbridge 150*25 5u; mobile phase: [water(0.04% NH₃H₂O+10 mM NH₄HCO₃)-ACN]; B %: 45%-65%, 10 min) to provide the title compound (10 mg, 15.06 μmol, 6.60% yield, 93% purity) as a white solid.

### Example 9. Preparation of (S)-N-(6-(3-cyanoguanidino)-2-oxo-1-phenoxyhexan-3-yl)-2-methoxy-2-methylpropanamide (9)

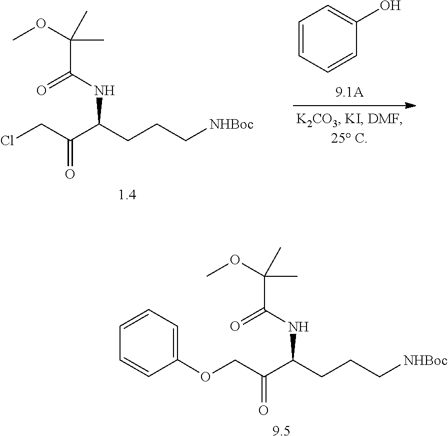

Compound 9.5: To a solution of compound 1.4 (2 g, 5.48 mmol, 1 eq), phenol 9.1A (515.87 mg, 5.48 mmol, 482.12 μL, 1 eq) in DMF (25 mL) was added KI (909.93 mg, 5.48 mmol, 1 eq) and K₂CO₃ (1.52 g, 10.96 mmol, 2 eq). The mixture was stirred at 25° C. for 12 hr. The reaction mixture was quenched by addition of H₂O (25 mL), and then extracted with EtOAc (25 mL×3). The combined organic layers were washed with H₂O (25 mL×3) and saturated brine (25 mL), dried over Na₂SO₄, filtered, and concentrated under reduced pressure to provide a residue which was purified by MPLC (SiO₂, petroleum ether/ethyl acetate=10/1 to 1:1) to afford compound 9.5 (1.69 g, 4.00 mmol, 72.97% yield) as yellow oil.

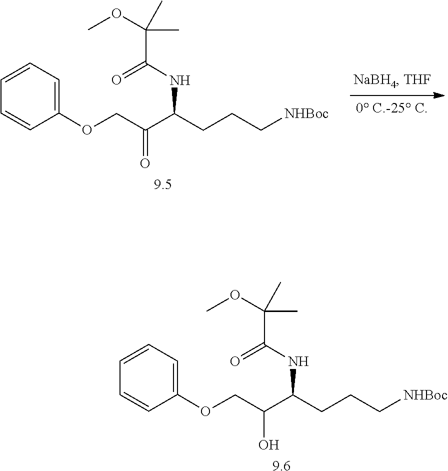

Compound 9.6: To a solution of compound 9.5 (1.69 g, 4.00 mmol, 1 eq) in THF (20 mL) was added NaBH₄ (378.31 mg, 10.00 mmol, 2.5 eq) at 0° C. The mixture was stirred at 25° C. for 12 hr. The reaction mixture was quenched by addition of H₂O (20 mL), and then extracted with EtOAc (20 mL×3). The combined organic layers were washed with saturated brine (20 mL), dried over Na₂SO₄, filtered, and concentrated under reduced pressure to provide a residue which was purified by MPLC (SiO₂, petroleum ether/ethyl acetate=10/1 to 0:1) to afford compound 9.6 (700 mg, 1.65 mmol, 41.22% yield) as a yellow oil.

¹H NMR (400 MHz, CHLOROFORM-d) δ 7.26-7.30 (m, 1 H), 6.84-7.08 (m, 4 H), 4.54-4.67 (m, 1 H), 3.94-4.03 (m, 2 H), 3.79-3.86 (m, 1 H), 3.23-3.30 (m, 3 H), 3.08-3.20 (m, 2 H), 1.66-1.75 (m, 1 H), 1.45-1.64 (m, 3 H), 1.42 (s, 9 H), 1.35-1.39 (m, 6 H).

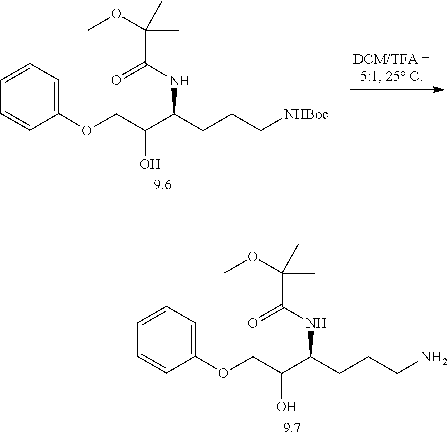

Compound 9.7: To a solution of compound 9.6 (350 mg, 824.44 μmol, 1 eq) in DCM (5 mL) was added TFA (1.54 g, 13.51 mmol, 1 mL, 16.38 eq). The mixture was stirred at 25° C. for 1 hr. The reaction mixture was concentrated under reduced pressure to afford compound 9.7 (670 mg, crude, TFA) as a yellow oil.

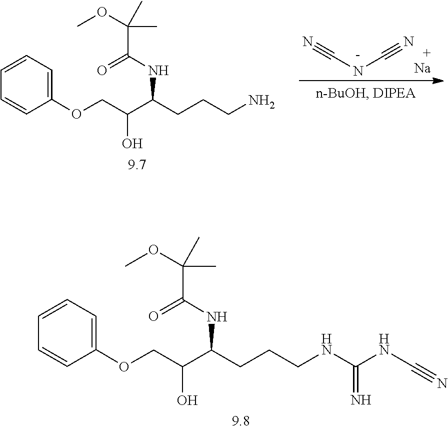

Compound 9.8: To a solution of compound 9.7 (670 mg, 1.53 mmol, 1 eq, TFA) in n-BuOH (25 mL) was added (dicyanoamino) sodium (149.66 mg, 1.68 mmol, 1.1 eq) and DIPEA (197.50 mg, 1.53 mmol, 266.18 μL, 1 eq). The mixture was stirred at 120° C. for 0.5 hr. The reaction mixture was concentrated under reduced pressure to provide compound 9.8 (920 mg, crude) as a yellow oil.

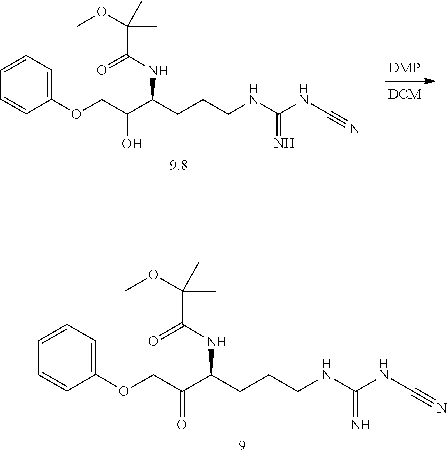 [SMILES: COC(C)(C)C(=O)N[C@@H](CCCNC(=N)NC#N)C(O)COc1ccccc1 ; "9.8" ; COC(C)(C)C(=O)N[C@@H](CCCNC(=N)NC#N)C(=O)COc1ccccc1 ; "9"]

Compound 9: To a solution of compound 9.8 (450 mg, 1.15 mmol, 1 eq) in DCM (20 mL) was added Dess-Martin periodinane (1.22 g, 2.87 mmol, 889.72 μL, 2.5 eq) at 0° C. The mixture was stirred at 25° C. for 2 hr. The reaction mixture was quenched by addition of Na₂SO₃ (20 mL), and then extracted with DCM (20 mL×3). The combined organic layers were washed with NaHCO₃ (20 mL×3) and saturated brine (20 mL), dried over Na₂SO₄, filtered, and concentrated under reduced pressure to provide a residue which was purified by pre-HPLC (column: Nano-micro Kromasil C18 100*30 mm Sum; mobile phase: [water (0.1% TFA)-ACN]; B %: 25%-40%, 10 min) to afford the title compound (5.88 mg, 14.55 umol, 1.27% yield, 96.4% purity) as a yellow oil.

LCMS (ESI): m/z: [M+H] calcd for C₁₉H₂₈N₅O₄:390; found 390; RT=2.749min. ¹H NMR (400 MHz, CHLOROFORM-d), δ 7.69 (br s, 1 H), 7.30-7.40 (m, 2 H), 7.01-7.10 (m, 1 H), 6.89 (br d, J=7.94 Hz, 2 H), 5.15-5.17 (m, 1 H), 4.59-4.75 (m, 2 H), 3.32 (s, 3 H), 3.04-3.30 (m, 2 H), 2.00-2.03 (m, 1 H), 1.66-1.75 (m, 1 H), 1.50-1.63 (m, 2 H), 1.37 (br d, J=12.57 Hz, 6 H).

### Example 10. Preparation of (S)-N-(6-(3-cyanoguanidino)-1-(4-fluorophenoxy)-2-oxohexan-3-yl)-2-methoxy-2-methylpropanamide (10)

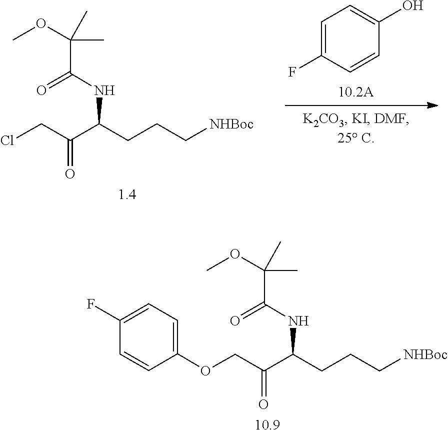

Compound 10.9: To a solution of compound 1.4 (1 g, 2.74 mmol, 1 eq), compound 10.2A (307.24 mg, 2.74 mmol, 1 eq) in DMF (10 mL) was added KI (454.97 mg, 2.74 mmol, 1 eq) and K₂CO₃ (757.58 mg, 5.48 mmol, 2 eq). The mixture was stirred at 25° C. for 12 hr. The reaction mixture was quenched by addition of H₂O (10 mL), and then extracted with EtOAc (10 mL×3). The combined organic layers were washed with H₂O (10 mL×3) and saturated brines, dried over Na₂SO₄, filtered and concentrated under reduced pressure to provide a residue which was purified by column chromatography (SiO₂, petroleum ether/ethyl acetate=10/1 to 1:1) to afford compound 10.9 (620 mg, 1.41 mmol, 51.35% yield) as a yellow oil.

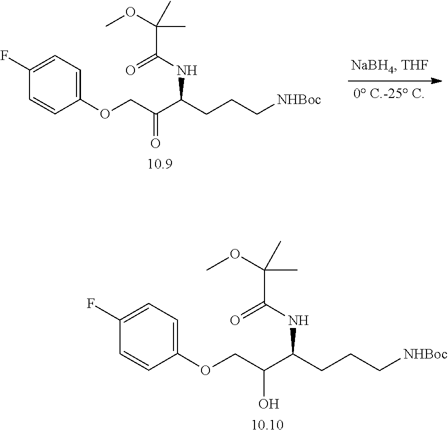

Compound 10.10: To a solution of compound 10.9 (620 mg, 1.41 mmol, 1 eq) in THF (10 m L) was added NaBH₄ (133.12 mg, 3.52 mmol, 2.5 eq) at 0° C. The mixture was stirred at 25° C. for 12 hr. The reaction mixture was quenched by addition of H₂O (20 mL), and then extracted with EtOAc (20 mL×3). The combined organic layers were washed with saturated brine (20 mL), dried over Na₂SO₄, filtered and concentrated under reduced pressure to provide a residue which was purified by MPLC (SiO₂, petroleum ether/ethyl acetate=10/1 to 0:1) to afford compound 10.10 (269 mg, 607.88 μmol, 43.19% yield) as a yellow oil.

¹H NMR (400 MHz, CHLOROFORM-d) 6.91-7.03 (m, 2 H), 6.76-6.88 (m, 2 H), 4.55-4.58 (m, 1 H), 4.06 (br d, J=4.19 Hz, 1 H), 3.90-4.01 (m, 2 H), 3.72-3.81 (m, 1 H), 3.24-3.31 (m, 3 H), 3.04-3.23 (m, 2 H), 1.67-1.77 (m, 1 H), 1.47-1.64 (m, 3 H), 1.43 (s, 9 H), 1.35-1.39 (m, 6 H).

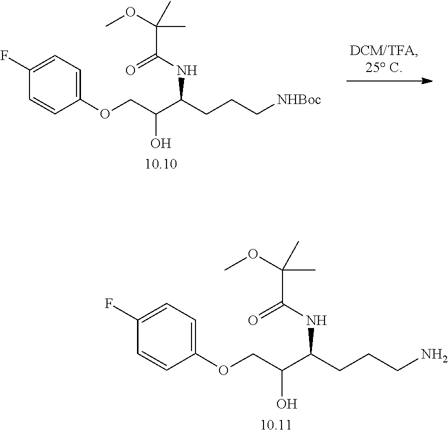

Compound 10.11: To a solution of compound 10.10 (269 mg, 607.88 μmol, 1 eq) in DCM (5 mL) was added TFA (1.54 g, 13.51 mmol, 1 mL, 22.22 eq). The mixture was stirred at 25° C. for 1 hr. The reaction mixture was concentrated under reduced pressure to afford compound 10.11 (550 mg, crude, TFA) as a yellow oil.

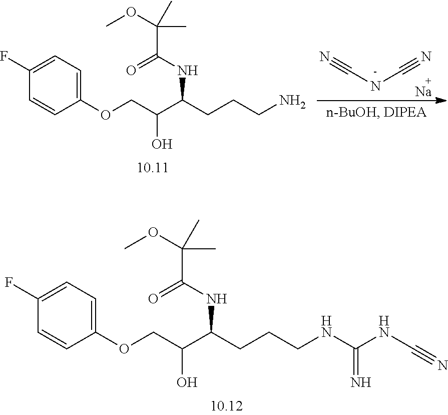

Compound 10.12: To a solution of compound 10.11 (550 mg, 1.21 mmol, 1 eq, TFA) in n-BuOH (22 mL) was added (dicyanoamino) sodium (118.01 mg, 1.33 mmol, 1.1 eq) and DIPEA (155.74 mg, 1.21 mmol, 209.89 μL, 1 eq). The mixture was stirred at 120° C. for 0.5 hr. The reaction mixture was concentrated under reduced pressure to afford compound 10.12 (810 mg, crude) as a yellow oil.

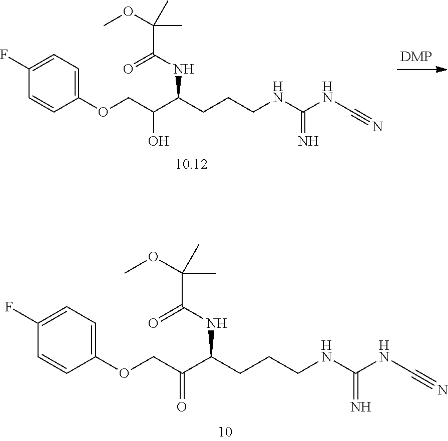 [SMILES: COC(C)(C)C(=O)N[C@@H](CCCNC(=N)NC#N)C(O)COc1ccc(F)cc1 ; COC(C)(C)C(=O)N[C@@H](CCCNC(=N)NC#N)C(=O)COc1ccc(F)cc1]

Compound 10: To a solution of compound 10.12 (400 mg, 976.91 μmol, 1 eq) in DCM (40 mL) was added Dess-Martin periodinane (1.04 g, 2.44 mmol, 756.11 μL, 2.5 eq) at 0° C. The mixture was stirred at 25° C. for 2 hr. The reaction mixture was quenched by addition of Na₂SO₃ (40 mL), and then extracted with DCM (40 mL×3). The combined organic layers were washed with NaHCO₃ (40 mL×3) and saturated brine 40 mL, dried over Na₂SO₄, filtered and concentrated under reduced pressure to provide a residue which was purified by pre-HPLC(column: Luna C18 100*30 5u; mobile phase: [water(0.1%TFA)-ACN]; B %: 10%-40%, 14 min) to afford the title compound (11.94 mg, 26.08 μmol, 2.67% yield, 89% purity) as a yellow oil.

LCMS (ESI): m/z: [M+H] calcd for C₁₉H₂₇FN₅O₄: 408; found 408; RT=3.220 min. ¹H NMR (400 MHz, CHLOROFORM-d) δ 7.66 (br s, 1 H), 7.03 (br t, J=8.49 Hz, 2 H), 6.85 (br dd, J=8.93, 4.08 Hz, 2 H), 5.07-5.10 (m, 1 H), 4.57-4.72 (m, 2 H), 3.49 (br s, 2 H), 3.32 (s, 3 H), 1.93-2.02 (m, 1 H), 1.75-1.90 (m, 2 H), 1.69-1.73 (m 1 H), 1.37 (d, J=13.45 Hz, 6 H)

### Example 11. Preparation of (S)-N-(6-(3-cyanoguanidino)-1-(3-fluorophenoxy)-2-oxohexan-3-yl)-2-methoxy-2-methylpropanamide (11)

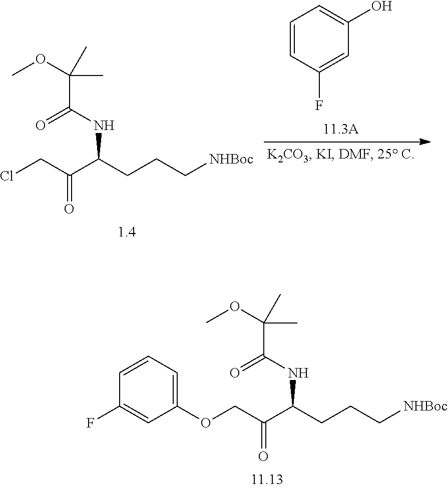 [SMILES: COC(C)(C)C(=O)N[C@@H](CCCNC(=O)OC(C)(C)C)C(=O)CCl ; Oc1cccc(F)c1 ; COC(C)(C)C(=O)N[C@@H](CCCNC(=O)OC(C)(C)C)C(=O)COc1cccc(F)c1]

Compound 11.13: To a solution of compound 1.4 (1 g, 2.74 mmol, 1 eq) and 3-fluorophenol 11.3A (307.16 mg, 2.74 mmol, 251.77 μL, 1 eq) in DMF (10 mL) was added KI (454.97 mg, 2.74 mmol, 1 eq) and K₂CO₃ (757.58 mg, 5.48 mmol, 2 eq). The mixture was stirred at 25° C. for 12 hr. The reaction mixture was quenched by addition of H₂O (10 mL), and then extracted with EtOAc (10 mL×3). The combined organic layers were washed with H₂O (10 mL×3) and saturated brine, dried over Na₂SO₄, filtered, and concentrated under reduced pressure to provide a residue which was purified by column chromatography (SiO₂, petroleum ether/ethyl acetate=10/1 to 1:1) to afford compound 11.13 (620 mg, 1.41 mmol, 51.37% yield) as yellow oil.

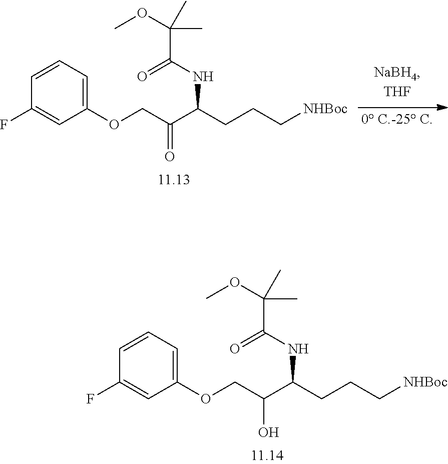 [SMILES: COC(C)(C)C(=O)N[C@@H](CCCNC(=O)OC(C)(C)C)C(=O)COc1cccc(F)c1 ; COC(C)(C)C(=O)N[C@@H](CCCNC(=O)OC(C)(C)C)C(O)COc1cccc(F)c1]

Compound 11.14: To a solution of compound 11.13 (620 mg, 1.41 mmol, 1 eq) in THF (10 mL) was added NaBH₄ (133.12 mg, 3.52 mmol, 2.5 eq) at 0° C. The mixture was stirred at 25° C. for 12 hr. The reaction mixture was quenched by addition of H₂O (10 mL), and then extracted with EtOAc (10 mL×3). The combined organic layers were washed with saturated brine (10 mL), dried over Na₂SO₄, filtered and concentrated under reduced pressure to afford compound 11.14 (586 mg, 1.32 mmol, 94.09% yield) as a yellow oil.

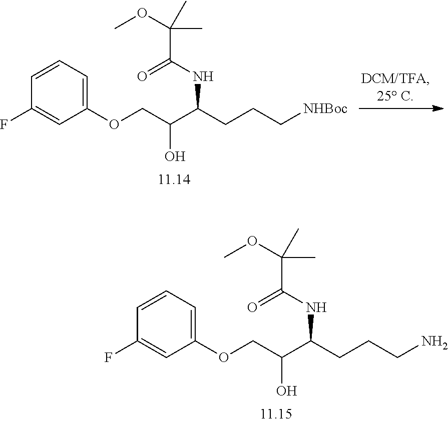 [SMILES: COC(C)(C)C(=O)N[C@@H](CCCNC(=O)OC(C)(C)C)C(O)COc1cccc(F)c1 ; COC(C)(C)C(=O)N[C@@H](CCCN)C(O)COc1cccc(F)c1]

Compound 11.15: To a solution of compound 11.14 (586 mg, 1.32 mmol, 1 eq) in DCM (8 mL) was added TFA (150.99 mg, 1.32 mmol, 98.05 μL, 1 eq). The mixture was stirred at 25° C. for 1 hr. The reaction mixture was concentrated under reduced pressure to afford compound 11.15 (920 mg, crude, TFA) as a yellow oil.

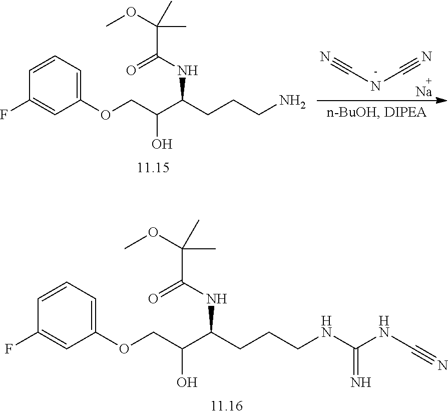

Compound 11.16: To a solution of compound 11.15 (400 mg, 876.37 μmol, 1 eq, TFA) and (dicyanoamino) sodium (85.83 mg, 964.01 μmol, 1.1 eq) in n-BuOH (16 mL) was added DIPEA (113.26 mg, 876.37 μmol, 152.65 μL, 1 eq). The mixture was stirred at 120° C. for 0.5 hr. The reaction mixture was concentrated under reduced pressure. The resulting residue was purified by pre-HPLC (column: Phenomenex Luna C18 200*40 mm*10 um; mobile phase: [water (0.1% TFA)-ACN]; B %: 25%-65%, 12 min) to afford compound 11.16 (38 mg, 92.81 μmol, 10.59% yield) as a yellow oil.

¹H NMR (400 MHz, CHLOROFORM-d) δ 7.50-7.61 (m, 1 H), 7.44 (br s, 1 H), 6.58-6.77 (m, 4 H), 3.93-4.27 (m, 5 H), 3.32 (d, J=3.53 Hz, 3 H), 3.10-3.22 (m, 2 H), 1.63-1.68 (s, 3 H), 1.50-1.55 (br s, 1 H), 1.38 (br d, J=4.19 Hz, 6 H).

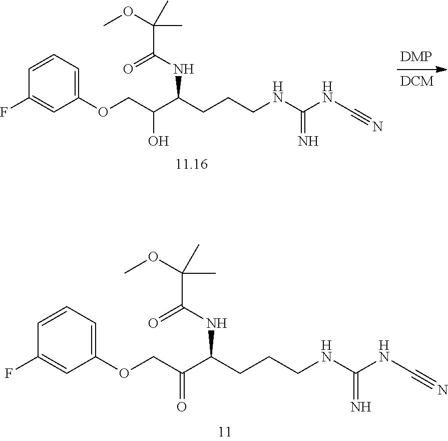 [SMILES: COC(C)(C)C(=O)N[C@@H](CCCNC(=N)NC#N)C(O)COc1cccc(F)c1 ; COC(C)(C)C(=O)N[C@@H](CCCNC(=N)NC#N)C(=O)COc1cccc(F)c1]

Compound 11: To a solution of compound 11.16 (38 mg, 92.81 μmol, 1 eq) in DCM (4 mL) was added Dess-Martin periodinane (98.41 mg, 232.02 μmol, 71.83 μL, 2.5 eq). The mixture was stirred at 25° C. for 12 hr. The reaction mixture was quenched by addition Na₂SO₃ (5 mL), and then extracted with DCM (5 mL×3). The combined organic layers were washed with NaHCO₃ (5 mL×3) and saturated brine (5 mL), dried over Na₂SO₄, filtered and concentrated under reduced pressure to provide a residue which was purified by prep-HPLC (column: Xtimate C18 150*25 mm*5 um; mobile phase: [water (10 mM NH₄HCO₃)-ACN]; B %: 30%-60%, 10 min) to provide the title compound (6.97 mg, 15.40 μmol, 16.59% yield, 90% purity) as a yellow solid.

LCMS (ESI): m/z: [M+H] calcd for C₁₉H₂₇FN₅O₄: 408; found 408; RT=2.475min. ¹H NMR (400 MHz, METHANOL-d4) δ ppm 7.22-7.32 (m, 1 H), 6.65-6.80 (m, 3 H), 4.86-4.90 (m, 2 H), 4.65 (dd, J=9.59, 4.30 Hz, 1 H), 4.58 (s, 1 H), 3.29-3.30 (m, 3 H), 3.19 (br t, J=6.84 Hz, 2 H), 1.91-2.04 (m, 1 H), 1.50-1.75 (m, 3 H), 1.23-1.44 (m, 6 H).

### Example 12. Preparation of (S)-2-acetamido-N-((S)-6-guanidino-1-((1,1,1,3,3,3-hexafluoropropan-2-yl)oxy)-2-oxohexan-3-yl)-3-methylbutanamide (12)

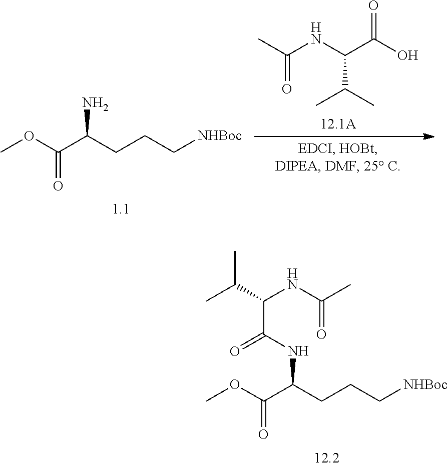 [SMILES: COC(=O)[C@@H](N)CCCNC(=O)OC(C)(C)C ; CC(=O)N[C@H](C(=O)O)C(C)C ; COC(=O)[C@H](CCCNC(=O)OC(C)(C)C)NC(=O)[C@@H](NC(C)=O)C(C)C]

Compound 12.2: To a solution of compound 12.1A (9.69 g, 60.90 mmol, 1 eq) in DMF (250 mL) was added HOBt (12.34 g, 91.35 mmol, 1.5 eq) and EDCI (17.51 g, 91.35 mmol, 1.5 eq) at 0° C. The mixture was stirred at 25° C. for 1 hr. Then compound 1.1 (15 g, 60.90 mmol, 1 eq), DIPEA (23.61 g, 182.70 mmol, 31.82 mL, 3 eq) was added to the mixture. The mixture was stirred at 25° C. for 12 hr. The reaction mixture was quenched by addition of H₂O (150 ml) at 25° C., and then extracted with ethyl acetate (150 mL×3). The combined organic layers were washed with H₂O (150 mL×3) and saturated brine (150mL), dried over Na₂SO₄, filtered and concentrated under reduced pressure to provide a residue which was purified by MPLC (SiO₂, petroleum ether/ethyl acetate=10/1 to 0:1) to afford compound 12.2 (8 g, 20.65 mmol, 33.90% yield) as a white solid.

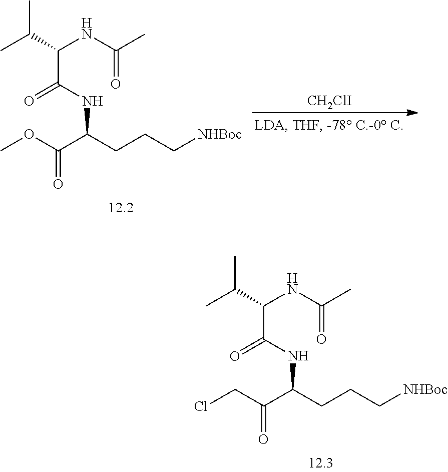 [SMILES: COC(=O)[C@H](CCCNC(=O)OC(C)(C)C)NC(=O)[C@@H](NC(C)=O)C(C)C ; "CH2ClI" ; CC(=O)N[C@H](C(=O)N[C@@H](CCCNC(=O)OC(C)(C)C)C(=O)CCl)C(C)C]

Compound 12.3: To a solution of DIPA (12.54 g, 123.92 mmol, 17.51 mL, 5.5 eq) in THF (120 mL) was added n-BuLi (2.5 M, 49.57 mL, 5.5 eq) at 0° C. The mixture was stirred at 0° C. for 30 min. The mixture was added a solution of compound 12.2 (8.73 g, 22.53 mmol, 1 eq) and chloro(iodo)methane (21.86 g, 123.92 mmol, 8.99 mL, 5.5 eq) in THF (160 mL) at −78° C. The mixture was stirred at −78° C. for 3 hr. The reaction mixture was quenched by addition of H₂O (150 mL) at 25° C., and then extracted with ethyl acetate (150 mL×3). The combined organic layers were washed with H₂O (150 mL×3) and brine (150mL), dried over Na₂SO₄, filtered, and concentrated under reduced pressure to provide a residue which was purified by MPLC (SiO₂, petroleum ether/ethyl acetate=10/1 to 0:1) to afford compound 12.3 (7.59 g, 18.70 mmol, 82.99% yield) as a yellow solid.

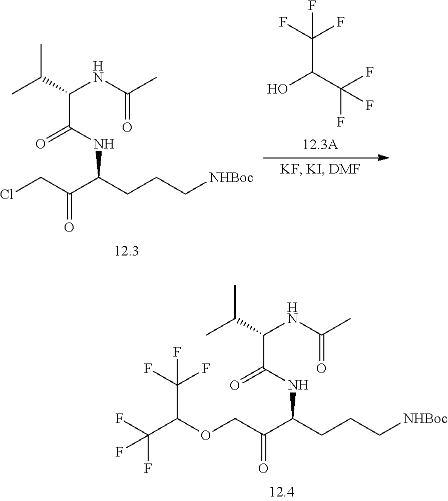 [SMILES: CC(=O)N[C@H](C(=O)N[C@@H](CCCNC(=O)OC(C)(C)C)C(=O)CCl)C(C)C ; OC(C(F)(F)F)C(F)(F)F ; CC(=O)N[C@H](C(=O)N[C@@H](CCCNC(=O)OC(C)(C)C)C(=O)COC(C(F)(F)F)C(F)(F)F)C(C)C]

Compound 12.4: To a solution of compound 12.3 (7.59 g, 18.70 mmol, 1 eq) and compound 12.3A (3.14 g, 18.70 mmol, 1 eq) in DMF (150 mL) was added KI (3.10 g, 18.70 mmol, 1 eq) and K₂CO₃ (5.17 g, 37.40 mmol, 2 eq). The mixture was stirred at 25° C. for 12 hr. The reaction mixture was quenched by addition of H₂O (100 mL) at 25° C., and then extracted with ethyl acetate (100 mL×3). The combined organic layers were washed with H₂O (100 mL×3), dried over Na₂SO₄, filtered, and concentrated under reduced pressure to provide a residue which was purified by column chromatography (SiO₂, petroleum ether/ethyl acetate=10/1 to 0:1) to afford compound 12.4 (1.08 g, 2.02 mmol, 10.78% yield) as a white solid.

¹H NMR (400 MHz, METHANOL-d₄) δ 5.04-4.97 (m, 1H), 4.81-4.64 (m, 2H), 4.50-4.39 (m, 1H), 4.13-4.08 (m, 1H), 3.09-3.02 (m, 2H), 2.00 (s, 3H), 1.91-1.75 (m, 1H), 1.66-1.46 (m, 4H), 1.44 (s, 9H), 0.98 (t, J=6.4 Hz, 6H).

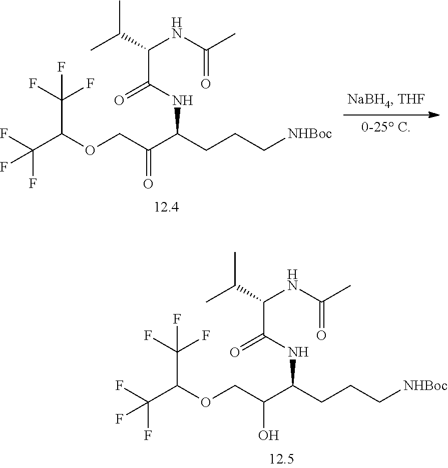 [SMILES: CC(=O)N[C@H](C(=O)N[C@@H](CCCNC(=O)OC(C)(C)C)C(=O)COC(C(F)(F)F)C(F)(F)F)C(C)C ; CC(=O)N[C@H](C(=O)N[C@@H](CCCNC(=O)OC(C)(C)C)C(O)COC(C(F)(F)F)C(F)(F)F)C(C)C]

Compound 12.5: To a solution of compound 12.4 (1.08 g, 2.02 mmol, 1 eq) in THF (20 mL) was added NaBH₄ (152.50 mg, 4.03 mmol, 2 eq) at 0° C. The mixture was stirred at 25° C. for 2 hr. The reaction mixture was quenched by addition of H₂O (20 ml), and then extracted with ethyl acetate (20 mL×3). The combined organic layers were washed with brine (20 mL), dried over Na₂SO₄, filtered, and concentrated under reduced pressure to provide a residue which was purified by column chromatography (SiO₂, petroleum ether/ethyl acetate=10/1 to 0:1) to afford compound 12.5 (593 mg, 1.10 mmol, 54.54% yield) as a white solid.

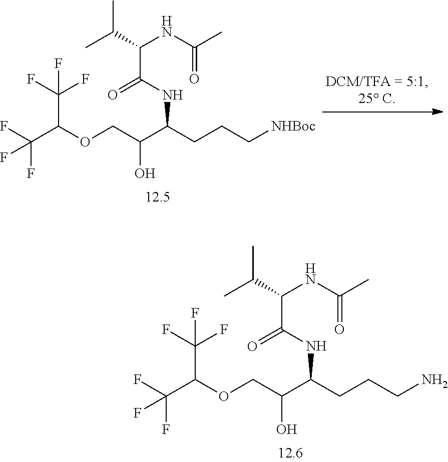 [SMILES: CC(=O)N[C@H](C(=O)N[C@@H](CCCNC(=O)OC(C)(C)C)C(O)COC(C(F)(F)F)C(F)(F)F)C(C)C ; CC(=O)N[C@H](C(=O)N[C@@H](CCCN)C(O)COC(C(F)(F)F)C(F)(F)F)C(C)C]

Compound 12.6: To a solution of compound 12.5 (593 mg, 1.10 mmol, 1 eq) in DCM (5 mL) was added TFA (1.54 g, 13.51 mmol, 1 mL, 12.29 eq). The mixture was stirred at 25° C. for 12 hrs. The reaction mixture was quenched by addition of H₂O 5 (mL), and then extracted with DCM (5 mL×3). The combined organic layers were washed with NaHCO₃ (5 mL×3) and saturated brine (100 mL), dried over Na₂SO₄, filtered and concentrated under reduced pressure to afford compound 12.6 (800 mg, crude) as a white solid and used to next step without purification.

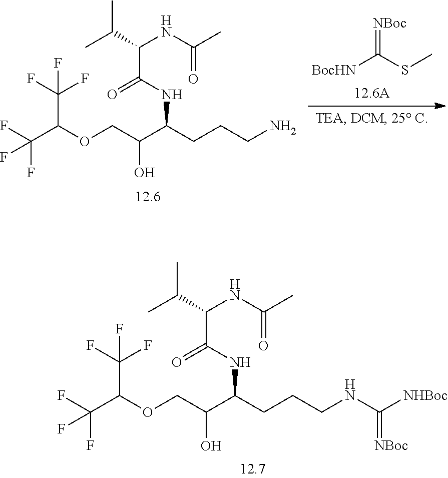

Compound 12.7: To a solution of compound 12.6 (530 mg, 957.69 μmol, 1 eq, TFA, calculated as 100% purity) in DCM (15 mL) was added TEA (290.73 mg, 2.87 mmol, 399.90 μL, 3 eq) and compound 12.6A (417.14 mg, 1.44 mmol, 1.5 eq). The mixture was stirred at 25° C. for 12 hr. The reaction mixture was quenched by addition of H₂O (20 mL), and then extracted with DCM (20 mL×3). The combined organic layers were washed with saturated brine (20 mL), dried over Na₂SO₄, filtered and concentrated under reduced pressure to provide a residue which was purified by column chromatography (SiO₂, petroleum ether/ethyl acetate=10/1 to 0:1) to afford compound 12.7 (100 mg, 146.70 μmol, 15.32% yield) as a white solid.

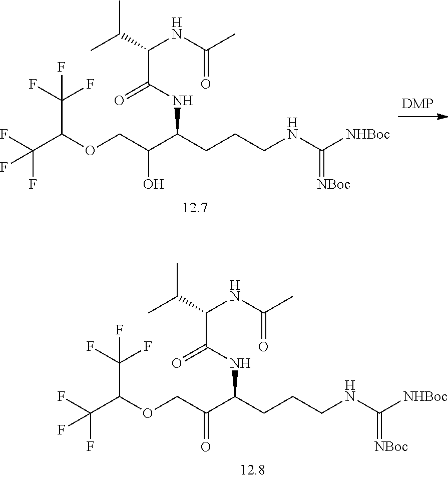

Compound 12.8: To a solution of compound 12.7 (100 mg, 146.70 μmol, 1 eq) in DCM (20 mL) was added Dess-Martin periodinane (155.55 mg, 366.75 μmol, 113.54 μL, 2.5 eq) at 0° C. The mixture was stirred at 25° C. for 12 hr. The reaction mixture was quenched by addition of H₂O (20 mL), and then extracted with DCM (20 mL×3). The combined organic layers were washed with brine 20 mL, dried over Na₂SO₄, filtered, and concentrated under reduced pressure to provide a residue which was purified by prep-HPLC (column: Xbridge 150*30 mm*10 um;mobile phase: [water(0.1% TFA)-ACN]; B %: 40%-70%,10 min) to afford compound 12.8 (20 mg, 29.43 umol, 20.06% yield) as a white solid.

¹H NMR (400 MHz, METHANOL-d₄) δ 5.06-4.99 (m, 1H), 4.79-4.63 (m, 2H), 4.54-4.44 (m, 1H), 4.07-3.99 (m, 1H), 3.49 (br t, J=6.7 Hz, 2H), 2.00 (s, 3H), 1.95-1.89 (m, 1H), 1.83-1.62 (m, 4H), 1.62-1.52 (m, 18H), 1.03-0.95 (m, 6H)

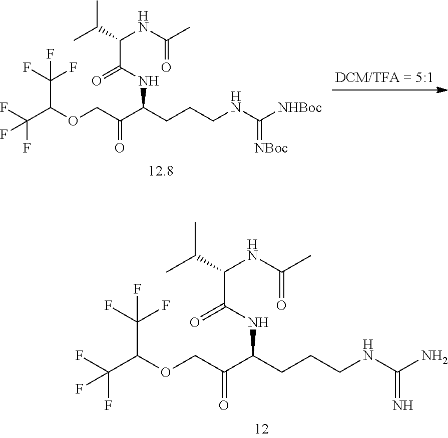

Compound 12: To a solution of compound 12.8 (41 mg, 60.33 μmol, 1 eq) in DCM (15 mL) was added TFA (4.62 g, 40.52 mmol, 3 mL, 671.66 eq). The mixture was stirred at 25° C. for 12 hr. The reaction mixture was concentrated under reduced pressure to provide the title compound (35 mg, crude) as a black oil.

LCMS (ESI): m/z: [M+H] calcd for C₁₇H₂₈F₆N₅O₄: 480; found 480; RT=1.528 min. ¹H NMR (400 MHz, METHANOL-d₄) δ 5.09-4.93 (m, 1H), 4.80-4.60 (m, 1H), 4.07-4.00 (m, 1H), 3.25-3.11 (m, 2H), 2.02-1.96 (m, 3H), 1.73-1.63 (m, 2H), 1.62-1.46 (m, 2H), 1.35-1.24 (m, 2H), 1.07-0.94 (m, 6H).

### Example 13. Inhibition of Arginine Gingipain

The capacities of compounds described above to inhibit the activity of RgpB were measured in a fluorogenic assay similar to those described in Barret Biochemical Journal. 1980, 187(3), 909. The specific assay conditions were as follows. Buffer: pH=7.5, 100 mM Tris-HCl, 75 mM NaCl, 2.5 mM CaCl₂, 10 mM cysteine, 1% DMSO after all additions. Protein: 0.02 nM RgpB, isolated from culture of Porphyromonas gingivalis, as described in Pike et al. J. Biol. Chem. 1994, 269(1), 406, and Potempa and Nguyen. Current Protocols in Protein Scienc. 2007, 21. 20. 1-21. 20. 27 . Fluorogenic substrate: 10 μM Boc-Phe-Ser-Arg-MCA. Time=90 minutes. Temperature=37° C. Each compound: 10 concentrations, starting at either 100 μM or 100 nM, with lower concentrations generated by serial 3-fold dilutions. By testing a range of concentrations for each compound, the concentration required to inhibit the activity of RgpB by 50% (the “IC₅₀”) was determined. All of examples 1-17 exhibited IC₅₀ values between around 20 picomolar and around 375 nM.

Advantageously, compounds according to the present disclosure were found to exhibit increased selectivity for Rgp over endogenous proteases such as cathepsins, in comparison to reference compound N-[(1S)-4-guanidino-1-[2-(2,3,5,6-tetrafluorophenoxy)acetyl]butyl]-cyclopentane carboxamide. For example, Compound 1 of Example 1 demonstrated IC₅₀ values over 10 μM when screened against cathepsins K, F, B, H, V, L, and S. The IC₅₀ values of Compound 1 were around 4 to 6 orders of magnitude higher for cathepsins K, F, and B than the IC₅₀ values of the reference compound for cathepsins K, F, and B. The decreased cathepsin inhibition activity of the compounds is advantageous because cathepsins are lysosomal proteases implicated in a number of important physiological processes including MHC-II-mediated antigen presentation, bone remodeling, keratinocyte differentiation, and prohormone activation. The compounds of the invention can therefore be used to selectively inhibit Rgp in a subject, resulting from invasive P. gingivalis, without perturbing endogenous cathepsin activity in the subject.

### VI. EXEMPLARY EMBODIMENTS

Exemplary embodiments provided in accordance with the presently disclosed subject matter include, but are not limited to, the claims and the following embodiments:
1. A compound according to Formula I:

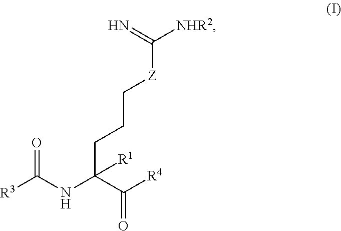

or a pharmaceutically acceptable salt thereof, wherein:

Z is selected from the group consisting of NH, CH₂, and S;

R¹ is selected from the group consisting of H and C₁₋₄ alkyl;

R² is selected from the group consisting of H, —CN, —OH, —OR^{2a}, —C(O)R^{2a}, and —C(O)OR^{2a};

R^{2a} is selected from the C₁₋₈ alkyl, C₆₋₁₀ aryl, and C₇₋₁₈ arylalkyl;

R³ is selected from the group consisting of C₃₋₈ cycloalkyl, C₃₋₈ alkyl, 3- to 12-membered heterocyclyl, C₆₋₁₀ aryl, and 5- to 12-membered heteroaryl, wherein R³ is optionally substituted with one or more R^{3a} substituents;

each R^{3a} is independently selected from the group consisting of halogen, —CN, —NO₂, —N₃, —OH, C₁₋₄ alkyl, C₁₋₄ haloalkyl, C₁₋₄ alkoxy, C₁₋₄ haloalkoxy, —N(R^{c})₂, —N⁺(R^{b})₃, —(CH₂)ₖC(O)R^{b}, —NR^{c}(CH₂)ᵤC(O)R^{b}, —O(CH₂)ᵤC(O)R^{b}, —(CH₂)ₖCONR^{c}R^{c}, —(CH₂)ₖNR^{c}C(O)R^{b}, —NR^{c}(CH₂)ᵤCONR^{c}R^{c}, —NR^{c}(CH₂)ᵤNR^{c}C—(O)R^{b}, —O(CH₂)ᵤCONR^{c}R^{c}, and —O(CH₂)ᵤNR^{c}C(O)R^{b}, and optionally substituted triazolyl;

each R^{b} is independently selected from the group consisting of C₁₋₄ alkyl, C₁₋₄ haloalkyl, and C₁₋₄ deuteroalkyl;

each R^{c} is independently selected from the group consisting of H and C₁₋₈ alkyl;

each subscript k is independently selected from 0, 1, 2, 3, 4, 5, and 6;

each subscript u is independently selected from 1, 2, 3, 4, 5, and 6;

R⁴ is selected from the group consisting of —CH₂R^{4a} and —CHS(O)(R^{4b})₂;

R^{4a} is selected from the group consisting of —O—R⁵, —SO—R⁶, 3- to 12-membered heterocyclyl, and 5- to 12-membered heteroaryl,
wherein 3- to 12-membered heterocyclyl is optionally substituted with one or more members independently selected from the group consisting of oxo, halogen, C₁₋₄ alkyl, and C₁₋₄ haloalkyl, and
5- to 12-membered heteroaryl is optionally substituted with one or more members independently selected from the group consisting of halogen, C₁₋₄ alkyl, and C₁₋₄ haloalkyl;

each R^{4b} is independently selected C₁₋₈ alkyl; and
R⁵ and R⁶ are selected from the group consisting of phenyl, C₁₋₈ alkyl, C₁₋₈ haloalkyl, and 5- to 12-membered heteroaryl,
wherein phenyl is optionally substituted with 1-5 halogens, and
wherein 5- to 12-membered heteroaryl is optionally substituted with one or more halogen, C₁₋₄ alkyl, or C₁₋₄ haloalkyl;

provided that R⁴ is other than 2,3,5,6-tetrafluorophenoxymethyl.
2. The compound of embodiment 1, or a pharmaceutically acceptable salt thereof, wherein R² is —CN.
3. The compound of embodiment 1, or a pharmaceutically acceptable salt thereof, wherein R² is selected from the group consisting of —OR^{2a}, —C(O)R^{2a}, and —C(O)OR^{2a}.
4. The compound of embodiment 1, or a pharmaceutically acceptable salt thereof, wherein R² is H.
5. The compound of any one of embodiments 1-4, having a structure according to Formula Ia:

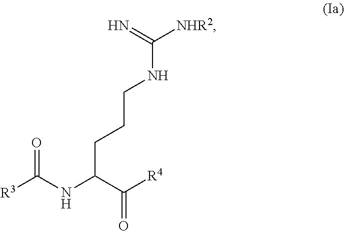

or a pharmaceutically acceptable salt thereof.
6. The compound of any one of embodiments 1-5, or a pharmaceutically acceptable salt thereof, wherein R⁴ is —CH₂OR⁵ and R⁵ is C₁₋₈ haloalkyl.
7. The compound of any one of embodiments 1-5, or a pharmaceutically acceptable salt thereof, wherein R⁴ is —CH₂OR⁵ and R⁵ is 5- to 12-membered heteroaryl, which is optionally substituted with one or more halogen, C₁₋₄ alkyl, or C₁₋₄ haloalkyl.
8. The compound of any one of embodiments 1-5, or a pharmaceutically acceptable salt thereof, wherein R⁴ is —CH₂OR⁵ and R⁵ is phenyl, which is optionally substituted with 1-5 halogens.
9. The compound of any one of embodiments 1-8, or a pharmaceutically acceptable salt thereof, wherein R³ is C₃₋₈ alkyl.
10. The compound of embodiment 9, or a pharmaceutically acceptable salt thereof, wherein R³ is substituted with R^{3a}, and R^{3a} is C₁₋₄ alkoxy.
11. The compound of any one of embodiments 1-8, or a pharmaceutically acceptable salt thereof, wherein R³ is selected from the group consisting of C₃₋₈ cycloalkyl and 5- to 12-membered heteroaryl.
12. The compound of embodiment 1, which is any of the species according to Formula la as set forth herein, or a pharmaceutically acceptable salt thereof.
13. The compound of embodiment 1, which is any of the species according to Formula Ib as set forth herein, or a pharmaceutically acceptable salts thereof.
14. A pharmaceutical composition comprising a compound of any one of embodiments 1-13, or a pharmaceutically acceptable salt thereof, and a pharmaceutically acceptable excipient.
15. A method of treating a disease or condition associated with P. gingivalis infection, the method comprising administering to a subject in need thereof an effective amount of a compound according to any one of embodiments 1-13, or a pharmaceutically acceptable salt thereof, or an effective amount of a composition according to embodiment 14.
16. The method of embodiment 15, wherein the disease or condition is selected from the group consisting of a brain disorder, periodontal disease, diabetes, a cardiovascular disease, arthritis, elevated risk of preterm birth, pneumonia, cancer, a kidney disease, a liver disease, a retinal disorder, and glaucoma.
17. The method of embodiment 15, wherein the disease or condition is a brain disorder.
18. The method of embodiment 16 or embodiment 17, wherein the brain disorder is selected from the group consisting of Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, cerebrovascular disease, Lewy Body disease, multiple system atrophy, schizophrenia, and depression.
19. The method of any one of embodiments 16-18, further comprising administering to the subject one or more active agents selected from the group consisting of a cholinesterase inhibitor, a serotonin modulator, an NMDA modulator, an Aβ targeted therapy, an ApoE targeted therapy, a microglia targeted therapy, a blood brain barrier targeted therapy, a tau targeted therapy, a complement targeted therapy, and an anti-inflammatory.
20. The method of any one of embodiments 15-19, wherein the compound is administered to the subject for at least one month.
21. The method of embodiment 20, wherein the compound is administered to the subject for at least one year.
22. A method for inhibiting an arginine gingipain, the method comprising contactin the arginine gingipain with an effective amount of a compound accoding to any one of embodiments any one of embodiments 1-13.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A compound according to Formula I: 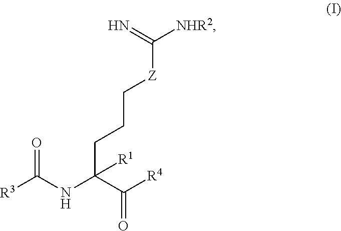
or a pharmaceutically acceptable salt thereof, wherein:
Z is selected from the group consisting of NH, CH₂, and S;
R¹ is selected from the group consisting of H and C₁₋₄ alkyl;
R² is selected from the group consisting of H, —CN, —OH, —OR^{2a}, —C(O)R^{2a}, and —C(O)OR^{2a};
R^{2a} is selected from the C₁₋₈ alkyl, C₆₋₁₀ aryl, and C₇₋₁₈ arylalkyl;
R³ is selected from the group consisting of C₃₋₈ alkyl which is substituted with R^{3a}, unsubstitued C₃₋₈ alkyl, C₃₋₈ cycloalkyl, 3- to 12-membered heterocyclyl, C₆₋₁₀ aryl, and 5- to 12-membered heteroaryl,
wherein C₃₋₈ cycloalkyl, 3- to 12-membered heterocyclyl, C₆₋₁₀ aryl, and 5- to 12-membered heteroaryl are optionally substituted with one or more R^{3a} substituents;
each R^{3a} is independently selected from the group consisting of C₁₋₄ alkoxy, halogen, —CN, —NO₂, —N₃, OH, C₁₋₄ alkyl, C₁₋₄ haloalkyl, C₁₋₄ haloalkoxy, —N(R^{c})₂, —N⁺(R^{b})₃, —(CH₂)ₖC(O)R^{b}, —NR^{c}(CH₂)ᵤC(O)R^{b}, —O(CH₂)ᵤC(O)R^{b}, —(CH₂)ₖCONR^{c}R^{c}, —(CH₂)ₖNR^{c}C(O)R^{b}, —NR^{c}(CH₂)ᵤCONR^{c}R^{c}, —NR^{c}(CH₂)ᵤNR^{c}C—(O)R^{b}, —O(CH₂)ᵤCONR^{c}T^{c}, and —O(CH₂)ᵤNR^{c}C(O)R^{b}, and optionally substituted triazolyl;
each R^{b} is independently selected from the group consisting of C₁₋₄ alkyl, C₁₋₄ haloalkyl, and C₁₋₄ deuteroalkyl;
each R^{c} is independently selected from the group consisting of H and C₁₋₈ alkyl;
each subscript k is independently selected from 0, 1, 2, 3, 4, 5, and 6;
each subscript u is independently selected from 1, 2, 3, 4, 5, and 6;
R⁴ is selected from the group consisting of —CH₂R^{4a} and —CHS(O)(R^{4b})₂;
R^{4a} is selected from the group consisting of —O—R⁵, —SO—R⁶, 3- to 12-membered heterocyclyl, and 5- to 12-membered heteroaryl,
wherein 3- to 12-membered heterocyclyl is optionally substituted with one or more members independently selected from the group consisting of oxo, halogen, C₁₋₄ alkyl, and C₁₋₄ haloalkyl, and
5- to 12-membered heteroaryl is optionally substituted with one or more members independently selected from the group consisting of halogen, C₁₋₄ alkyl, and C₁₋₄ haloalkyl;
each R^{4b} is independently selected C₁₋₈ alkyl; and
R⁵ and R⁶ are selected from the group consisting of C₃₋₈ haloalkyl, C₁₋₂ haloalkyl, phenyl, C₁₋₈ alkyl, and 5- to 12-membered heteroaryl,
wherein phenyl is optionally substituted with 1-5 halogens, and
wherein 5- to 12-membered heteroaryl is optionally substituted with one or more halogen, C₁₋₄ alkyl, or C₁₋₄ haloalkyl;
provided that R⁴ is other than 2,3,5,6-tetrafluorophenoxymethyl.

2. The compound of claim 1, or a pharmaceutically acceptable salt thereof, wherein R² is H or —CN.

3. (canceled)

4. The compound of claim 1, or a pharmaceutically acceptable salt thereof, wherein R² is selected from the group consisting of —OR^{2a}, —C(O)R^{2a}, and —C(O)OR^{2a}.

5. The compound of claim 1, having a structure according to Formula Ia: 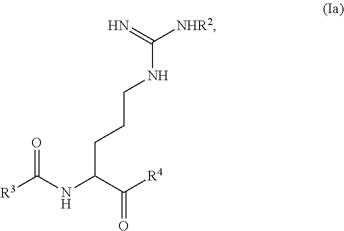
or a pharmaceutically acceptable salt thereof.

6. The compound of claim 1, or a pharmaceutically acceptable salt thereof, wherein R⁴ is —CH₂OR⁵ and R⁵ is C₃₋₈ haloalkyl.

7. The compound of claim 1, or a pharmaceutically acceptable salt thereof, wherein R⁴ is —CH₂OR⁵ and R⁵ is 5- to 12-membered heteroaryl, which is optionally substituted with one or more halogen, C₁₋₄ alkyl, or C₁₋₄ haloalkyl.

8. The compound of claim 1, or a pharmaceutically acceptable salt thereof, wherein R⁴ is —CH₂OR⁵ and R⁵ is phenyl, which is optionally substituted with 1-5 halogens.

9. The compound of claim 1, or a pharmaceutically acceptable salt thereof, wherein R³ is C₃₋₈ alkyl substituted with R^{1a}, and R^{3a} is C₁₋₄ alkoxy.

10. The compound of claim 1, or a pharmaceutically acceptable salt thereof, wherein R³ is unsubstituted C₃₋₈ alkyl.

11. The compound of claim 1, or a pharmaceutically acceptable salt thereof, wherein R³ is selected from the group consisting of C₃₋₈ cycloalkyl and 5- to 12-membered heteroaryl.

12. The compound of claim 1, which is selected from the group consisting of: 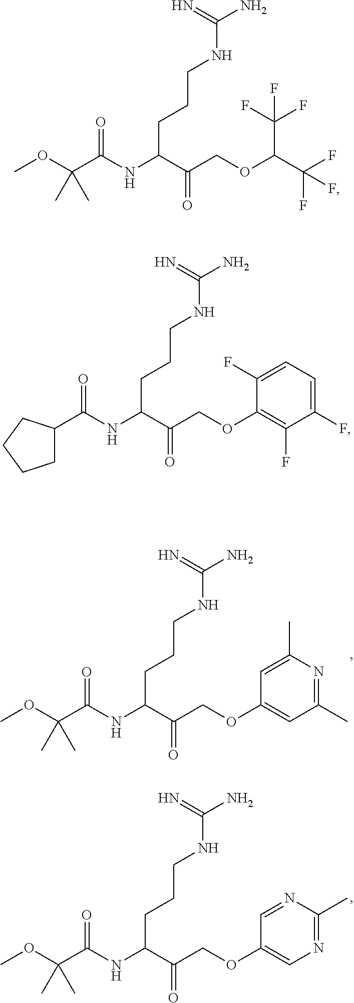 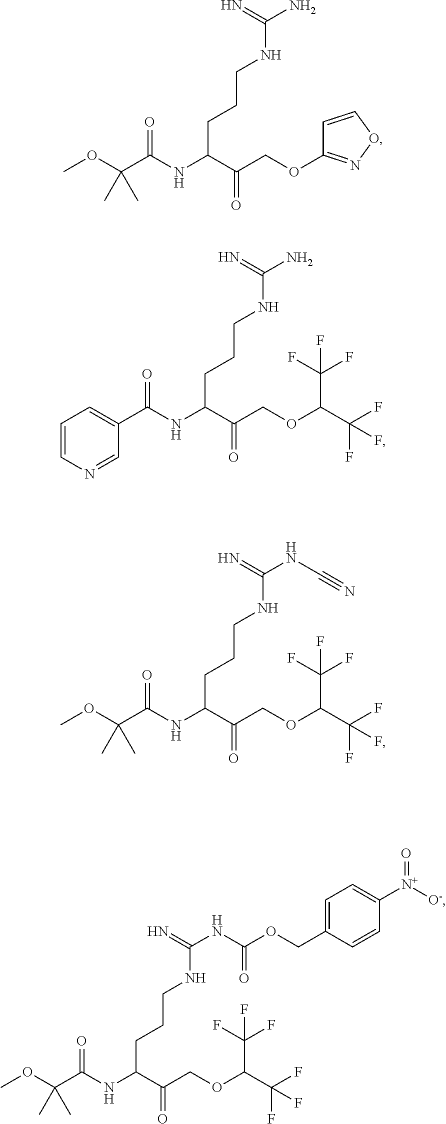 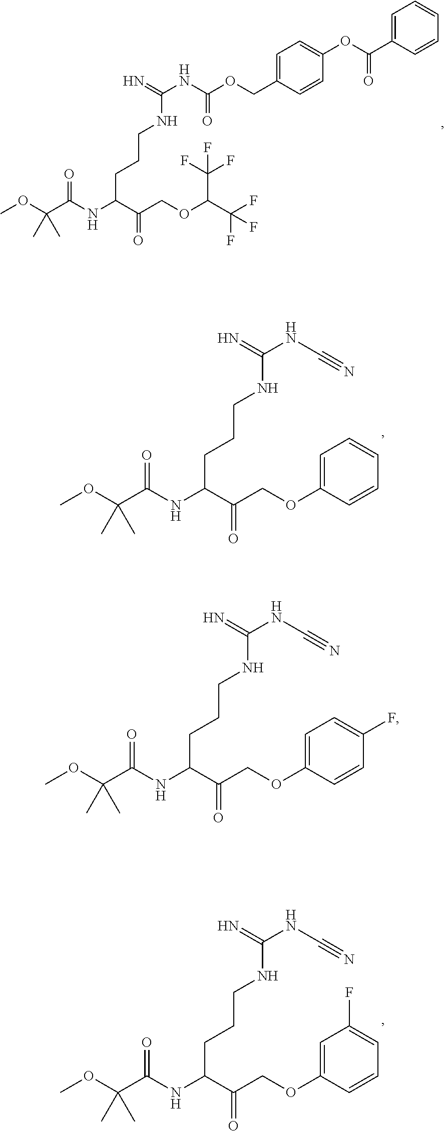 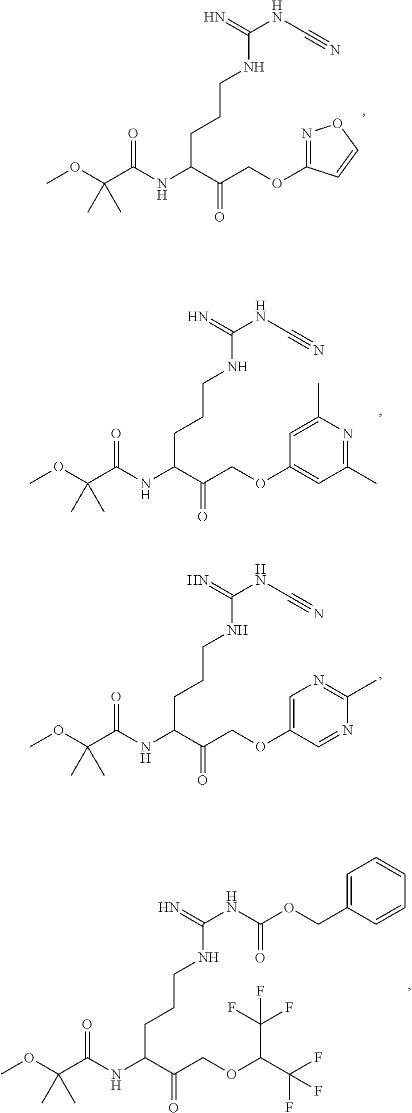 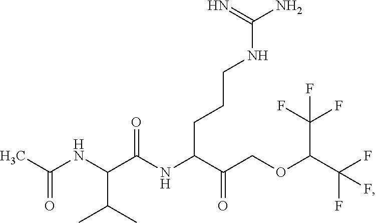
and pharmaceutically acceptable salts thereof.

13. The compound of claim 1, which is selected from the group consisting of: 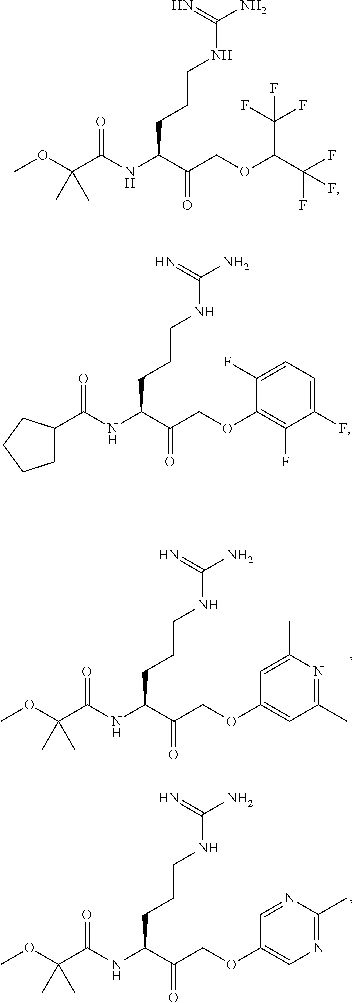 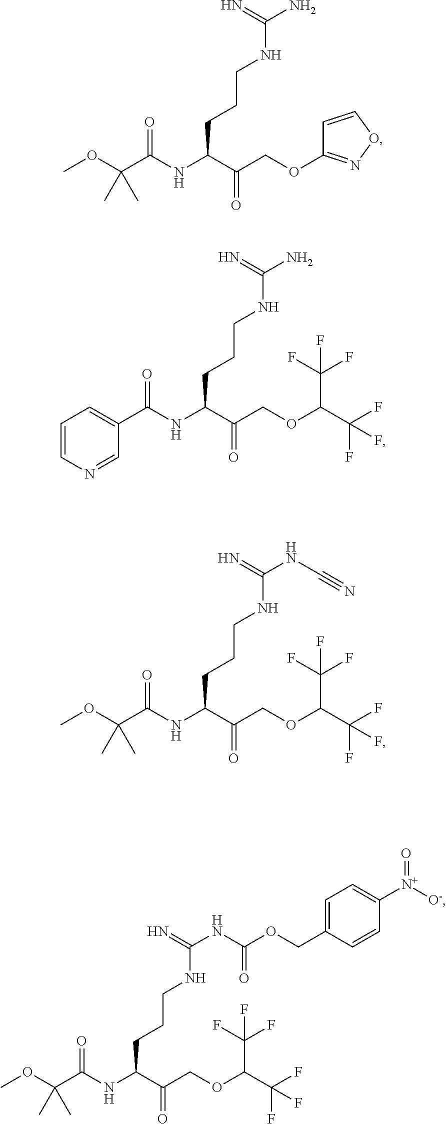 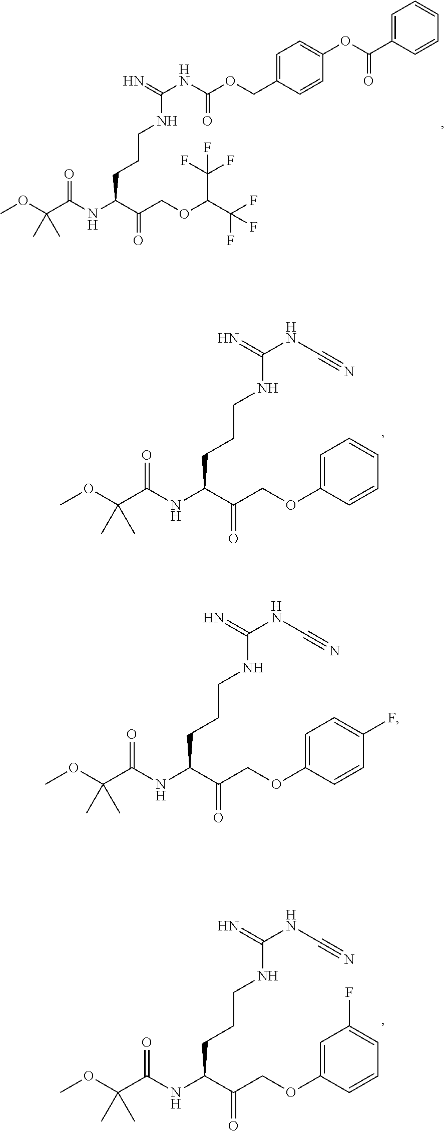 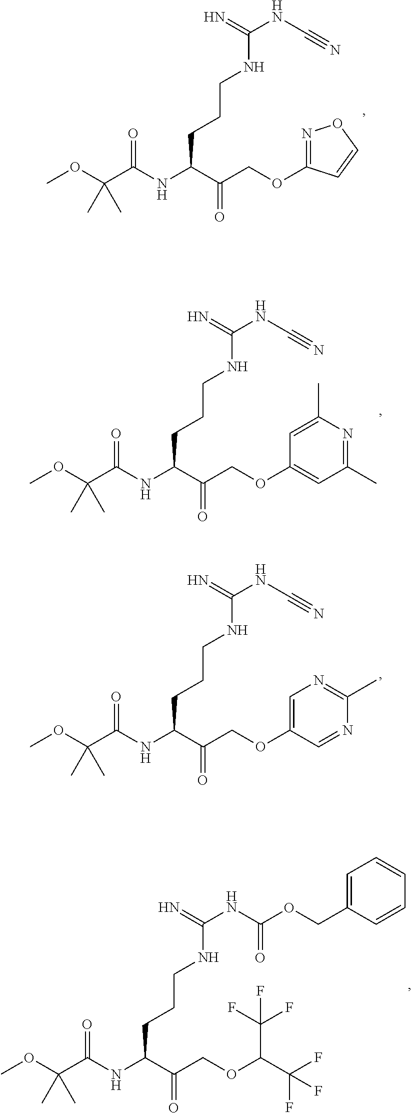 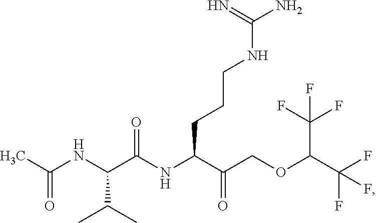
and pharmaceutically acceptable salts thereof.

14. A pharmaceutical composition comprising a compound of claim 1, or a pharmaceutically acceptable salt thereof, and a pharmaceutically acceptable excipient.

15. A method of treating a disease or condition associated with P. gingivalis infection, the method comprising administering to a subject in need thereof an effective amount of a compound according to claim 1, or a pharmaceutically acceptable salt thereof.

16. The method of claim 15, wherein the disease or condition is selected from the group consisting of a brain disorder, periodontal disease, diabetes, a cardiovascular disease, arthritis, elevated risk of preterm birth, pneumonia, cancer, a kidney disease, a liver disease, a retinal disorder, and glaucoma.

17. The method of claim 15, wherein the disease or condition is a brain disorder.

18. The method of claim 16, wherein the brain disorder is selected from the group consisting of Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, cerebrovascular disease, Lewy Body disease, multiple system atrophy, schizophrenia, and depression.

19. The method of claim 16, further comprising administering to the subject one or more active agents selected from the group consisting of a cholinesterase inhibitor, a serotonin modulator, an NMDA modulator, an Al3 targeted therapy, an ApoE targeted therapy, a microglia targeted therapy, a blood brain barrier targeted therapy, a tau targeted therapy, a complement targeted therapy, and an anti-inflammatory.

20. The method of claim 15, wherein the compound is administered to the subject for at least one month or at least one year.

21. (canceled)

22. A method for inhibiting an arginine gingipain, the method comprising contacting the arginine gingipain with an effective amount of a compound according to claim 1.
